(19) United States
(12) Patent Application Publication
(10) Pub. No.: US 2021/0190797 A1
(43) Pub. Date: Jun. 24, 2021

(54) Methods and reagents for diagnosis of SARS-CoV-2 infection

(71) Applicants: EUROIMMUN Medizinische Labordiagnostika AG, Luebeck (DE); Charité - Universitätsmedizin Berlin, Berlin (DE)

(72) Inventors: Claudia MESSING, Klempau (DE); Katja Steinhagen, Gross Groenau (DE); Erik Lattwein, Luebeck (DE); Konstanze Stiba, Rostock (DE); Fabian Lindhorst, Luebeck (DE); Eva Neugebauer, Zittau (DE); Marcel Müller, Berlin (DE); Victor Corman, Berlin (DE)

(73) Assignees: EUROIMMUN Medizinische Labordiagnostika AG, Luebeck (DE); Charité - Universitätsmedizin Berlin, Berlin (DE)

(21) Appl. No.: 17/180,616

(22) Filed: Feb. 19, 2021

(30) Foreign Application Priority Data
Feb. 19, 2020 (EP) 20158348.1
Feb. 20, 2020 (EP) 20158626.0
Feb. 21, 2020 (EP) 20158821.7
Aug. 20, 2020 (DE) 20 2020 003 564.5
Aug. 28, 2020 (DE) 20 2020 104 982.8

(51) Int. Cl. G01N 33/68; G01N 33/543

(52) U.S. Cl. CPC G01N 33/6857; G01N 2800/12; G01N 33/54326; G01N 33/6893

(57) ABSTRACT

A method diagnoses a SARS-CoV-2 infection and includes detecting the presence or absence of an antibody to SEQ ID NO: 1, preferably IgA class antibody, in a sample from a subject. A method differentially diagnoses a coronavirus infection. An antibody to SEQ ID NO: 1, preferably IgA class antibody, is used for diagnosing a SARS-CoV-2 infection or for the differential diagnosis of a coronavirus infection, preferably for distinguishing between a SARS-CoV-2, MERS and NL63, 229E, OC43 and HKU1 infection. A kit includes a polypeptide comprising SEQ ID NO: 1 or a variant thereof, preferably coated to a diagnostically useful carrier and one or more of the following reagents: an antibody to SEQ ID NO: 1, a washing buffer, a means for detecting the presence of an antibody, preferably IgA class antibody, preferably a secondary antibody binding specifically to IgA class antibodies, preferably comprising a detectable label, and a dilution buffer.

### CROSS-REFERENCE TO RELATED APPLICATIONS

This application claims priority to German utility model registration No. 20 2020 003, 564.5 filed on Aug. 20, 2020, German utility model registration No. 20 2020 104 982.8 filed on Aug. 28, 2020, European patent application No. 20158348.1 filed on Feb. 19, 2020, European patent application No. 20158626.0 filed on Feb. 20, 2020, and European patent application No. 20158821.7 filed on Feb. 21, 2020. The content of each of these applications is hereby incorporated by reference in its entirety.

### REFERENCE TO A SEQUENCE LISTING

The present application is accompanied by an ASCII text file as a computer-readable form containing the sequence listing entitled, “003250US_SL_ST25.txt”, created on Feb. 17, 2021, with the file size of 174,970 bytes, the content of which is hereby incorporated by reference in its entirety.

### BACKGROUND OF THE INVENTION

### Field of the invention

The present invention relates to a method for diagnosing a SARS-CoV-2 infection comprising the step of detecting the presence or absence of an antibody to SEQ ID NO: 1, preferably IgA class antibody, in a sample from a subject, a method for the differential diagnosis of a coronavirus infection, a use of an antibody to SEQ ID NO: 1, preferably IgA class antibody for diagnosing a SARS-CoV-2 infection or for the differential diagnosis of a coronavirus infection, preferably for distinguishing between a SARS-CoV-2, MERS, NL63, 229E, 0043 and HKU1 infection, and a kit comprising a polypeptide comprising SEQ ID NO: 1 or a variant thereof, preferably coated to a diagnostically useful carrier and one or more, preferably all reagents from the group comprising an antibody to SEQ ID NO: 1, a washing buffer, a means for detecting the presence of an antibody, preferably IgA class antibody, preferably a secondary antibody binding specifically to IgA class antibodies, preferably comprising a detectable label, and a dilution buffer.

### Description of Related Art

At the end of 2019, a rising number of pneumonia patients with unknown pathogen emerged from Wuhan, the capital of Hubei province, China, to nearly the entirety of China. A novel coronavirus was isolated and based on its phylogeny, taxonomy and established practice, the Coronavirus Study Group (CSG) recognized it as a sister to severe acute respiratory syndrome coronavirus (SARS-CoV-1) and labeled it as severe acute respiratory syndrome coronavirus 2 (SARS-CoV-2). Although SARS-CoV-2 is generally less pathogenic than SARS-CoV-1 and Middle East respiratory syndrome coronavirus (MERS-CoV), it has a relatively high transmissibility. Since symptoms may be mild and may be confused with a cold, there is the danger that patients may be unaware that they have been infected and may help the virus spread further.

SARS-CoV-2 is a coronavirus which has four major structural proteins, specifically the spike (S), envelope (E), membrane (M) and nucleocapsid (N) proteins. The N protein holds the RNA genome, while the other three structural proteins are components of the viral envelope. The S protein is responsible for allowing the virus to attach and fuse to the membrane of a host cell. It comprises an S1 domain which mediates the attachment and an S2 domain which mediates the fusion of the viral cellular membrane with the host cell. The S1 domain comprises the receptor binding domain (RBD), the binding site to the receptor angiotensin converting enzyme 2 (ACE2) on human host cells. Therefore, the RBD is a binding site of neutralizing antibodies which block the interaction between the virus and its host cells, thus conferring immunity. By contrast to SARS-CoV-1 and SARS-CoV-2, which are associated with a high mortality and severe illness, other coronaviruses exist which are associated with a mild and passing illness, such as coronaviruses 229E, NL63, OC43 and HKU1. These coronaviruses are frequently associated with common cold, in particular among children.

Owing to the high transmissibility and health impact, reliable methods for the diagnosis of SARS-CoV-2 infection are paramount. In the past, a combination of serological and real-time reverse transcription-polymerase chain reaction (RT-PCR) methods were used to detect and confirm infections with coronaviruses.

RT-PCR methods are based on the detection of one or more nucleic acids of the coronavirus of interest, more specifically of the viral ribonucleic acid (RNA). RT-PCR-based methods are rapid and, based on samples from throat or nasal pharyngeal swabs, can be used during the first week of illness for the detection of SARS-CoV-2. However, their usefulness very much depends on how long the nucleic acid is detectable in samples, which varies from virus to virus. In particular, a negative result from a diagnostic test performed on a sample obtained at an early stage of the disease may be meaningless. Moreover, generally a sample from the upper respiratory tract of the patient is required. Improper or insufficient recovery of such a sample, prolonged transportation time and associated degradation of the viral RNA, or instrument malfunction may also lead to false-negative results. Therefore, several samples should be examined, one from week 1 of the infection and a follow-up sample obtained 3 to 4 weeks later.

Corman et al. published a real-time RT-PCR based assay for the detection of SARS-CoV-2 (Carman V M, Landt O, Kaiser M, et al. Detection of 2019 novel coronavirus (2019-nCoV) by real-time RT-PCR. Euro SurveilL 2020;25(3):2000045. doi:10.2807/1560-7917.ES.2020.25.3.2000045), first published in January 2020 by the WHO on their webpage.

Serological tests are also frequently used. These tests are based on the detection of antibodies directed against certain virus-specific antigens. However, serological assays also suffer from severe impediments:

One frequently observed issue is their limited sensitivity. In order for serological assays to yield a positive result, the presence of virus-antigen specific antibodies in the patient's blood is required. Generally, the time point from which an antibody is first produced and detectable after an infection with a new virus cannot be predicted, because it depends on a multitude of factors, including the virus itself, the viral load i.e., the quantity of virus in a given amount of patient's blood, patient-related factors such as age, gender, health condition, immune status, etc., the immunoglobulin class of the antibody of interest, and the antigen target chosen to set up a diagnostic test.

Particularly at very early stages of the disease, i.e. before the patient's body has been able to mount a specific antibody response in detectable quantities, serological assays may give a negative result, despite an ongoing infection. Further serological testing, taking into account whether an antibody to be detected belongs to a certain immunoglobulin (Ig) class, may help monitor the course of the disease and distinguish an acute from a past infection or a vaccination. Most serological assays currently available for detection of viral infections including SARS-CoV-1 infection hence focus on the detection of IgG, or IgG and IgM class antibodies. However, besides their risk of yielding false-negative results specifically in very early stages of infection due to the intrinsic delay of antibody response, serological assays for diagnosing SARS-CoV-1 infection are also prone to false-positive results, presumably due to a high seroprevalence in the population of antibodies against common seasonal coronaviruses (CoV) and the associated presence of cross-reactive antibodies against conserved parts of immunogenic virus proteins. This cross-reactivity against such antigenically closely related seasonal coronaviruses also disallowed a differential diagnosis, for example a differentiation of the life-threatening variant SARS-CoV-1 from other coronaviruses. Challenges and pitfalls of serological assays for diagnosing and differentiating coronaviruses, specifically for diagnosing SARS-CoV-1, have been reviewed in Meyer, Drosten & Willer, Virus Research 194 (2014); 175-186. These multiple challenges associated with the serological diagnosis of earlier coronaviruses, e.g, poor sensitivity in early stages of infection; and poor specificity caused by the presence of cross-reactive antibodies, also apply to the development of diagnostic assays for detection of the recently emerged severe acute respiratory syndrome coronavirus 2 (SARS-CoV-2).

In view of the current severe threat posed by the ongoing global SARS-CoV-2 pandemic and the above mentioned issues of available assays for diagnosing a corresponding infection, there is an urgent need in the art for improved, in particular more sensitive, more specific, and hence more reliable means and methods for diagnosing a SARS-CoV-2 infection, particularly for the early diagnosis of a SARS-CoV-2 infection, and for differentiation thereof from infections with other coronaviruses, such as the common seasonal coronaviruses, in order to enable a more targeted virus-specific and hence more effective treatment.

Previous assays for diagnosing coronavirus infections are described, for example, in the following documents: WO2014/045254 discloses assays for diagnosing a Middle East respiratory syndrome-related coronavirus (MERS-CoV) infection, including serological assays wherein antibodies against viral proteinaceous antigens are detected in a sample. However, no practical evidence of such an assay is shown. US2005/0112559 discloses SARS-CoV-1-related methods and agents. The nucleocapsid (N) protein is considered the major diagnostic antigen. WO2005/118813 discloses a variant of SARS-CoV-1-related S protein and the detection of its presence, or the presence of antibodies binding to it, in samples. US2006/0188519 discloses peptides for the diagnosis of a SARS-CoV-1 infection. Hsueh et al. reported that IgG could be detected as early as four days after the onset of a SARS-CoV-1 infection, simultaneously as or one day earlier than IgM and IgA (Hsue, P. R., Huang, L. M., Chen, P. J., Kao, C. L., and Yang P. C. (2004) Chronological evolution of IgM, IgA, IgG and neutralization antibodies after infection with SARS-associated coronavirus, Clinical Microbiology and Infection, 10(12), 1062-1066). Reusken et al. disclose the specific, but not necessarily sensitive detection of antibodies to a SARS-CoV-2 spike protein fragment (Specific serology for emerging human coronaviruses by protein microarray. Euro Surveill. 2013;18(14). Yu et al. (2020) Measures for diagnosing and treating infections by a novel coronavirus responsible for a pneumonia outbreak originating in Wuhn, China, Microbes and Infection 22 (2020), 74J9 disclose, without pointing to specific antigens, that immunological methods may be used for detecting a SARS-CoV-2 infection, but are associated with poor sensitivity and specificity.

### SUMMARY OF THE INVENTION

The present invention addresses this need by providing diagnostic means and methods overcoming these previous impediments.

Specifically, the present invention addresses, without intended to be limiting, the following problems:

One problem addressed by the present invention is to provide an assay and reagents for the early serological detection of SARS-CoV-2. This is particularly important, as patients may still suffer from active disease and be contagious, even if a negative PCR result was obtained. N.B. that the Center for Disease Control and Prevention (CDC) recommends that patients remain isolated for at least 10 days since symptom onset and up to 20 days in cases of severe illness.

Another problem addressed by the present invention is to provide an assay that may be used to distinguish known coronavirus infections, in particular a SARS-Colt-2 infection from other coronavirus infections, preferably coronaviruses associated with mild cold-like symptoms or other pathogens or causes of such symptoms. Preferably, the infections can be distinguished at an early stage.

Another problem addressed by the present invention is to provide an assay with high optimized reliability, in particular with regard to sensitivity and/or specificity, preferably sensitivity.

Another problem addressed by the present invention is to provide an assay with high sensitivity, for patients lacking antibodies to the SARS-CoV-2 N protein or lacking IgM class antibodies.

Another problem addressed by the present invention is to provide an assay with long-lasting high sensitivity.

The present invention includes the following embodiments;
1. A method for diagnosing a SARS-Col/-2 infection comprising the step of detecting the presence or absence of an antibody to SEQ ID NO: 1, preferably IgA class antibody, in a sample from a subject.
2. A method for the differential diagnosis of a coronavirus infection, preferably for distinguishing between a SARS-CoV; preferably SARS-CoV-2; MFRS, NL63, 229E, 0043 and HKU1 infection, comprising the step of detecting the presence or absence of an antibody to SEQ ID NO: 1 in a sample from a subject, preferably an IgA and/or IgG class antibody, more preferably an IgA class antibody.
3. The method according to any of embodiments 1 or 2, wherein the presence of an IgG and/or IgM class antibody to SEQ ID NO: 1 is detected in addition to an IgA class antibody to SEQ ID NO: 1.
4. The method according to any of embodiments 1 to 3, wherein the sample is a blood sample, preferably selected from the group comprising whole blood, serum or plasma.
5. The method according to any of embodiments 1 to 4, wherein an IgA class antibody to SEQ ID NO: 1 is detected using a labeled secondary antibody, preferably binding to IgA class antibodies.
6. The method according to any of embodiments 1 to 5, wherein the IgA antibody is detected using a method selected from the group comprising colorimetry, immunofluorescence, detection of enzymatic activity, chemiluminescence and radioactivity.
7. The method according to any of embodiments 1 to 6, wherein the infection is detected at an early stage.
8. A use of an antibody to SEQ ID NO: 1, preferably IgA class antibody, for diagnosing a SARS-CoV-2 infection or for the differential diagnosis of a coronavirus infection, preferably for distinguishing between a SARS-CoV, preferably SARS-CoV-2, MRS and NL63, 229E, 0C43 and HKU1 infection:
9. The use according to embodiment 8, wherein the use is for the early diagnosis of a SARS-CoV-2 infection.
10: A kit comprising a polypeptide comprising SEQ ID NO: 1 or a variant thereof, preferably coated to a diagnostically useful carrier and one or more, preferably all reagents from the group comprising an antibody to SEQ ID NO: 1, a washing buffer, a means for detecting the presence of antibody to SEQ ID NO: 1, preferably an IgA class antibody, preferably a secondary antibody binding specifically to IgA class antibodies, preferably comprising a detectable label, and a dilution buffer.
11. The kit according to embodiment 10, wherein the diagnostically useful carrier is selected from the group comprising a bead, preferably a paramagnetic bead, a test strip, a microtiter plate, a membrane, preferably from the group comprising western blot, line blot and dot blot, a lateral flow device, a glass surface, a slide, a microarray and a biochip and is preferably a microtiter plate.
12. The kit according to embodiments 10 or 11, wherein the kit comprises two or more, preferably three or more calibrators.
13. The kit according to embodiment 12, wherein each calibrator is a recombinant antibody binding to SEQ ID NO: 1 which is preferably recognized by a secondary antibody binding to IgA class antibodies.
14. A use of a polypeptide comprising SEQ ID NO: 1 or a variant thereof or an antibody to SEQ ID NO: 1, preferably an IgA class antibody, for the manufacture of a diagnostic kit.
15. A use of a recombinant antibody binding to SEQ ID NO: 1 which is preferably recognized by a secondary antibody binding to IgA class antibodies as a calibrator for the early diagnosis of a SARS-CoV-2 infection.

The above-discussed problems are solved by the present invention, as described in the present specification and in the embodiments.

### BRIEF DESCRIPTION OF THE DRAWINGS

FIG. 1 shows a time course of antibody levels to SEQ ID NO: 1 (IgA, IgG) and N protein (IgG, IgM) monitored in two patients (FIG. 1), as described in Example 3.

FIG. 2 shows monitoring of another time course with a patient infected with SARS-CoV-2. IgA class antibodies to SEQ ID NO: 1 (squares), IgG class antibodies to SEQ ID NO: 1 (triangles) and IgG class antibodies to N protein (circles) were determined.

FIG. 3A shows IFA using acetone-fixed S1-expressing or control plasmid-transfected HEK293 cells (A) incubated directly with anti-His tag (1:200) or patient serum (PS, 1:100) in the first step and anti-mouse-IgG-FITC or anti-human-IgG-FITC in the second step. S1-expressing cells had been transfected with a pTriEx vector expressing SEQ ID NO: 2 using standard methods. Representative cells identified as reactive with patient antibody were marked using arrows.

FIG. 3B shows IFA using acetone-fixed Si-expressing or control plasmid-transfected HEK293 cells incubated 30 min with PBS prior to the two-step incubation with PS3 as described in FIG. 3A. Si-expressing cells had been transfected with a pTriEx vector expressing SEQ ID NO: 2 using standard methods, Representative cells identified as reactive with patient antibody were marked using arrows.

FIG. 3C shows IFA using acetone-fixed S1-expressing or control plasmid-transfected HEK293 cells incubated 30 min with PBS, followed by an incubation with PS3 (1:100) in the first step, anti-human-IgG-Biotin (1:200) in the second step and ExtrAvidin-FITC (1:2000) in the third step. S1-expressing cells had been transfected with a pTriEx vector expressing SEQ ID NO: 2 using standard methods. Representative cells identified as reactive with patient antibody were marked using arrows.

FIG. 4 shows the detection of IgG, IgA and IgM antibodies from the sample of a patient based on dot blot analysis using a fusion protein comprising RBD.

FIG. 5 shows the detection of IgG, IgA and IgM antibodies from the sample of a patient based on dot blot analysis using a polypeptide comprising SEQ ID NO: 1.

FIG. 6 shows the detection of IgM antibodies from the sample of a patient based on dot blot analysis using fragments of SEQ ID NO: 1 and fusion proteins thereof.

FIG. 7 shows the detection of IgA antibodies from the sample of a patient based on dot blot analysis using fragments of SEQ ID NO: 1 and fusion proteins thereof.

FIG. 8 shows the detection of IgG antibodies from the sample of a patient based on dot blot analysis using fragments of SEQ ID NO: 1 and fusion proteins thereof.

FIG. 9 shows the time-dependent detection of IgM antibodies from samples from patients based on dot blot analysis using fragments of SEQ ID NO: 1 and fusion proteins thereof.

FIG. 10 shows the time-dependent detection of IgG antibodies from samples from patients based on dot blot analysis using fragments of SEQ ID NO: 1 and fusion proteins thereof.

FIG. 11 shows the time-dependent detection of IgA antibodies from samples from patients based on dot blot analysis using fragments of SEQ ID NO: 1 and fusion proteins thereof.

FIG. 12 shows the detection of IgA antibodies from the sample of a patient based on Western analysis using a polypeptide comprising RBD.

FIG. 13 shows the detection of IgM antibodies from the sample of a patient based on Western blot analysis using a polypeptide comprising RBD.

FIG. 14 shows the detection of IgG antibodies from the sample of a patient based on Western blot analysis using a polypeptide comprising RBD.

FIG. 15 shows the time-dependent detection of IgA antibodies from samples of patients based on Western blot analysis using a polypeptide comprising SEQ ID NO: 1.

FIG. 16 shows the time-dependent detection of IgM antibodies from samples of patients based on Western blot analysis using polypeptide comprising SEQ ID NO: 1.

FIG. 17 shows the time-dependent detection of IgG antibodies from samples of patients based on Western blot analysis using a polypeptide comprising SEQ ID NO: 1.

### DETAILED DESCRIPTION OF THE INVENTION

In a first aspect, the invention provides a method for diagnosing a SARS-CoV-2 infection, comprising the step of detecting the presence or absence of an antibody to SEQ ID NO: 1, preferably IgM, IgG and/or IgA class antibody, more preferably IgA class antibody, in a sample from a subject. Whenever reference to a SEQ ID NO is made throughout this specification and the SEQ ID NO denotes an amino acid sequence, it is intended to denote a polypeptide comprising said corresponding amino acid string even if in that connection the term “polypeptide” is not specifically mentioned. The term “comprising” has two meanings in connection with this invention, namely “containing” and “consisting of.”

In a second aspect, the invention provides a method for the differential diagnosis of a coronavirus infection, preferably for distinguishing between a SARS-CoV; preferably SARS-CoV-2; MERS, NL63, 229E, OC43 and HKU1 infection, comprising the step of detecting the presence or absence of an antibody to SEQ ID NO: 1 in a sample from a subject, preferably an IgA and/or IgG class antibody, more preferably an IgA class antibody.

In a preferred embodiment, the presence of an IgG and/or IgM class antibody to SEQ ID NO: 1 is detected in addition to an IgA class antibody to SEQ ID NO: 1.

In accordance with the present invention, the “subject”, or interchangeably referred to herein as “patient”, refers to a mammal, preferably a human, but may alternatively also refer to a different mammal, such as a non-human primate or other mammalian animal or even a non-mammalian animal capable of producing an antibody to SEQ ID NO: 1 or variant thereof.

In accordance with the present invention, the term “sample” from a subject may be a sample of any bodily fluid or tissue of said subject that may comprise an antibody. Exemplary bodily fluids include for example blood, saliva, nasal mucus or lymph fluid. In a preferred embodiment, the sample is a blood sample, preferably selected from the group comprising whole blood, serum, plasma, capillary blood, arterial blood, venous blood or any mixture thereof. The capillary blood is preferably in the form of a dried blot spot, which may be prepared by the patient, sent to the lab, followed by extraction of the blood. The skilled person is aware of various means and methods that may be applied to obtain a sample from a subject suitable for the purposes of the herein disclosed methods, products and uses and in the required quantities. In certain embodiments, the sample may be obtained from the subject by a physician, whereas in other cases, the sample may be obtained by the subject itself, for example, by using minimal invasive means, such as finger pricking to draw blood (“finger-stick blood”).

It is also understood that, for the purposes of the herein disclosed invention, the sample may originate from a single subject, i.e., a single individuum, but may alternatively also comprise samples from more than one subject, wherein said samples from more than one subject are pooled into a single sample. For example, in certain cases, it might be more efficient in terms of resources and experimental time, to first analyze a pooled sample comprising samples from a group of subjects (e.g. members of a common household, school class, sports team, or same company department), and only in case of the detection of the presence of an antibody to SEQ ID NO: 1 or a variant thereof, to conduct a second analysis wherein samples from individual subjects are assayed separately.

Various methods or uses according to the invention can be conducted with a sample from a subject as described herein. These methods or uses can also be characterized as “in vitro” methods or “in vitro” uses.

In accordance with the present invention, the term “secondary antibody” in its broadest sense is to be understood to refer to any kind of “binding moiety”, preferably binding protein, capable of specific binding to an IgA, IgG and/or IgM class antibody or a fragment thereof such as a constant domain of a particular Ig class of a selected species, preferably human species. Non-limiting examples of binding moieties include antibodies, for example antibodies immunologically or genetically derived from any species, for example human, chicken, camel, llama, lamprey, shark, goat, rodent, cow, dog, rabbit, etc., antibody fragments, domains or parts thereof, for example Fab, Fab′, F(ab′)₂, scFab, Fv, scFv, VH, VHH, VL, VLRs, and the like, diabodies, monoclonal antibodies (mAbs), polyclonal antibodies (pAbs), mAbdAbs, phage display-derived binders, affibodies, heteroconjugate antibodies, bispecific antibodies, evibodies, lipocalins, anticalins, affibodies, avimers, maxibodies, heat shock proteins such as GroEL and GroES, trans-bodies, DARPins, aptamers, C-type lectin domains such as tetranectins; human γ-crystallin and human ubiquitin-derived binders such as affilins, PDZ domain-derived binders; scorpion toxin and/or Kunitz-type domain binders, fibronectin-derived binders such as adnectins, receptors, ligands, lectins, streptavidin, biotin, including derivatives and/or combinations thereof such as bi-/multi-specific formats formed from two or more of these binding molecules. Various antibody-derived and alternative (La. non-antibody) binding protein scaffolds including methods of generation thereof are known in the art (e.g. reviewed in Chiu M L et al., Antibodies (Basel), (2019):8(4):55; Simeon R. & Chen Z., Protein Cell, (2018);9(1):3-14; and Chapter 7—Non-Antibody Scaffolds from Handbook of Therapeutic Antibodies (2007) edited by Stefan Dübel.

All these types of molecules are well known in the art. Some examples are described in more detail herein below.

“Evibodies” are engineered binding proteins derived from the variable(V)-set Ig-like scaffold of the T-cell surface receptor Cytotoxic T Lymphocyte-associated Antigen 4 (CTLA-4). Loops corresponding to CDRs of antibodies can be substituted with heterologous sequences to confer different binding properties. Methods of making Evibodies are known in the art and are described, for example, U.S. Pat. No. 7,166,697.

“Lipocalins” are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. “Anticalins”, also termed “Affilins”, in accordance with the present invention, are between 160-180 amino acids in size, and are derived from lipocalins (Rothe C & Skerra A., BioDrugs. (2018); 32(3):233-243; Gebauer M & Skerra A, Curr Opin Biotechnol. (2019); 60:230-241).

“Affibodies” are a family of antibody mimetics that is derived from the Z-domain of staphylococcal protein A. Affibodies are structurally based on a three-helix bundle domain. An affibody has a molecular mass of around 6 kDa and is stable at high temperatures and under acidic or alkaline conditions. Target specificity is obtained by randomization of amino acids located in two alpha-helices involved in the binding activity of the parent protein domain (Feldwisch, J & Tolmachev, V. (2012) Methods Mol. Biol. 899:103-126). Methods of making affibodies are known in the art and are described in Wikman M et al. Protein Eng Des Set (2004); 17(5):455-62.

“Avimers”, short for avidity multimers), are a class of artificial multi-domain proteins which specifically bind certain antigens via multiple binding sites. This protein is also known as “maxibody” or low-density lipoprotein receptor (LDLR) domain A. It consists of two or more (poly)peptide sequences, which are based on A domains, The A domains are 30 to 35 amino acids scaffolds (˜4 kDa) derived from extracellular cysteine-rich cell surface receptor proteins and stabilized by disulfide bond formation and complexation of calcium ions. The scaffold structure is maintained by 12 conserved amino acids, leaving all the remaining non-conserved residues amenable to randomization and ligand binding. Avimers are highly thermostable. Due to their small size, avimers often consist of multiple A-domains with each binding to a different site on the target, thereby achieving increased affinity through avidity (Silverman J et a!, (2005), Nat Biotechnol 23; 1556-1561),

“DARPins” are designed ankyrin repeat domains and based on tightly packed ankyrin repeats, each forming a β-turn and two antiparallel α-helices. DARPins usually carry three repeats corresponding to an artificial consensus sequence, whereby a single repeat typically consists of 33 amino acids, six of which form the binding surface. During recombinant library design, these sites are used to introduce the codons of random amino acids. DARPins are typically formed by two or three of the binding motifs contained between the N- and C-terminal motifs shielding the hydrophobic regions. DARPins are small proteins (˜14-18 kDa) that are extremely thermostable and resistant to proteases and denaturing agents (Pluckthun A., Annu Rev Pharmacal Toxicol. (2015); 55:489-511).

“Kunitz-type domain binders” are ˜60-amino-acid polypeptides (˜7 kDa) derived from the active motif of Kunitz-type protease inhibitors such as aprotinin (bovine pancreatic trypsin inhibitor), Alzheimer's amyloid precursor protein, and tissue factor pathway inhibitor. The hydrophobic core of the Kunitz domain is composed of a twisted two-stranded antiparallel β-sheet and two α-helices stabilized by three pairs of disulfide bonds. Residues in the three loops can be substituted without destabilizing the structural framework (Hasse R J et al. (2006). Protein Sci 15:14-27; Simeon R. & Chen Z. Protein Cell. (2018);9(1):3-14).

“Adnectins” is a class of binding proteins having a scaffold which consists of a backbone of the natural amino acid sequence of the 10th domain of the 15 repeating units of human fibronectin type III (FN3). The molecule adopts a β-sandwich fold with seven strands connected by six loops similar like an immunoglobulin domain, but without any disulfide bonds. Three loops at one end of the β-sandwich can be engineered to enable an adnectin to specifically recognize a target of interest. Non-loop residues have also been found to expand the available binding footprint. Ligand-binding adnectin variants with binding affinities in the nanomolar to picomolar range have been selected via mRNA, phage, and yeast display (Haskel B J, et al. (2008) J Mol Biol 381:1238-1252).

Means and methods for developing, screening and identification of suitable binding molecules of various scaffolds, including, without being limiting, those described herein above, toward desired target structures, such as the IgA and/or IgG class antibodies, are well known and routinely employed in the art. Exemplary nowadays routinely-performed methods include, without intended to being limiting, high-throughput (HT) combinatorial library-based display and selection methods, such as phage display, ribosome display, mRNA display, and cell surface display (e.g. yeast display).

In a preferred embodiment, the secondary antibody is an immunoglobulin (Ig), preferably IgG raised in a non-human species, wherein said secondary antibody specifically binds immunoglobulins of one or more specific Ig classes or fragments thereof (e.g. a constant domain of a particular Ig class) of another selected species, preferably human species. An example is a polyclonal antibody raised in goat that specifically recognizes human IgA (i.e., a polyclonal goat anti-human IgA antibody). In another preferred embodiment, the secondary antibody is a monoclonal antibody that specifically binds immunoglobulins of one or more specific Ig classes or fragments thereof (e.g. a constant domain of a particular Ig class) of another selected species, preferably human species. Means and methods for producing (mono- or polyclonal) antibodies capable of specific binding of one or more selected target antigens are well known in the art.

In certain embodiments, the secondary antibody may be chosen to specifically bind to only one, only two, or all three classes of Ig antibodies, i.e. IgA, IgG and/or IgM. In certain embodiments, instead of using only one single kind of secondary antibody, a mixture of several different secondary antibodies may be used, wherein the different secondary antibodies either bind to the same one or different Ig classes (e.g., a mixture of different antibodies (e.g., polyclonal antibodies) all binding to IgA), or to, e.g. IgA and IgG or IgM or wherein the different secondary antibodies bind to different individual target structures (e.g. one kind of secondary antibody specifically binding to IgA, and another specifically binding to IgG).

In a preferred embodiment, the antibody to SEQ ID NO: 1 is detected using a labeled secondary antibody, preferably a labeled secondary antibody binding to IgA, IgG and/or IgM class antibodies.

As used herein, the term “labeled”, with regard to the secondary antibody, is intended to embrace such embodiments wherein the secondary antibody is labelled by coupling, preferably physically linking, a detectable substance, such as a radioactive agent or a other molecule providing a detectable signal, such as, without intended to being limiting, a fluorophore, such as a small organic chemical fluorophore or fluorescent protein, or an enzymatically active label, i.e. an enzyme, such as alkaline phosphatase, whose presence can be assessed and optionally be quantified based on its reactivity with, and/or conversion of, a substrate substance. Various suitable detectable labels are known in the art and some of which are also described herein below,

In a preferred embodiment, the antibody, preferably IgA, IgG or IgM class antibody, is detected using a method selected from the group comprising colorimetry, immunofluorescence, detection of enzymatic activity, chemiluminescence and radioactivity.

In a preferred embodiment, the infection is detected at an early stage, In a preferred embodiment, the term “early stage”, as referred to herein in the context of the course of a SARS-CoV-2 infection, refers to, in such cases where the time-point of infection is known, e.g. infection of a subject in a laboratory experiment, any time point within less than 14 days, preferably less than six days after the time-point of infection, i.e. the time-point of initial contact between the virus and the subject's body. In another preferred embodiment, the term early stage“, as referred to herein in the context of the course of a SARS-CoV-2 infection, refers to any time-point within less than 14 days, preferably less than six days after the onset of illness (also termed in the field of virology as “days post onset of illness” (dpoi)”), i.e. after the first occurrence of one or more typical SAR-CoV-2 infection-associated clinical symptoms, such as defined herein. The person skilled in the art is aware that upon infection, components of a corona virus can be detected directly, for example using PCR for detecting nucleic acid and immunoassays for detecting virus antigens in samples. After the peak of the infection with the maximum virus load, possibly only few days after the first contact of the patient with the virus, the concentration of virus decreases which makes the direct detection increasingly difficult and finally impossible, at the latest when the virus is absent in samples. Meanwhile, as soon as virus components are present in the blood of the patient, the production of antibodies against virus components is triggered. In another preferred embodiment, the term “early stage” refers to the time window between the first contact between virus and patient or the onset of illness, preferably the onset of illness, and the production of detectable IgG antibodies to the virus, more preferably before IgG antibodies are the dominant immunoglobulin class, i.e. are present at a higher concentration than IgA and IgM, most preferably before the peak of IgG concentration is reached. Thus, the detection of IgA and IgM antibodies can be a contribution to the diagnosis of an infection that is still acute, with detectable symptoms or not, but before the full IgG immune response is developed.

In a 3^{rd} aspect, the invention provides a use of an antibody to SEQ ID NO: 1, preferably IgA class antibody, for diagnosing a SARS-CoV-2 infection or for the differential diagnosis of a coronavirus infection, preferably for distinguishing between a SARS-CoV, preferably SARS-CoV-2, MERS, NL63, 229E, 0043 and HKU1 infection.

In a preferred embodiment, the use is for the early diagnosis or early stage diagnosis of a SARS-CoV-2 infection.

In a 4^{th} aspect, the invention provides a kit comprising a polypeptide comprising SEQ ID NO: 1 or a variant thereof, preferably coated to a diagnostically useful carrier and one or more, preferably all reagents from the group comprising an antibody to SEQ ID NO: 1, a washing buffer, a means for detecting the presence or absence of an antibody to SEQ ID NO: 1, preferably an IgA, IgG and/or IgM class antibody, more preferably an IgA class antibody, preferably a secondary antibody binding specifically to IgA IgG and/or IgM, more preferably IgA class antibodies, preferably comprising a detectable label, and a dilution buffer.

In a preferred embodiment, the diagnostically useful carrier is selected from the group comprising a bead, preferably a magnetic or paramagnetic bead, a test strip, a microtiter plate, a membrane, preferably from the group comprising a nitrocellulose membrane, western blot, line blot and dot blot, a lateral flow device, a glass surface, a slide, a microarray, a chromatography column and a biochip and is preferably a microtiter plate.

In a preferred embodiment, the kit comprises two or more, preferably three or more calibrators.

In a preferred embodiment, each calibrator is a recombinant antibody binding to SEQ ID NO: 1 which is preferably recognized by a secondary antibody binding to IgA class antibodies. In certain preferred embodiments, the calibrator is a recombinant antibody binding to SEQ ID NO: 1 or variant thereof, and said recombinant antibody is preferably an immunoglobulin (Ig) of the same Ig class as the Ig class to which the secondary antibody binds.

In a 5^{th} aspect, the invention provides a use of a polypeptide comprising SEQ ID NO:

1 or a variant thereof and/or an antibody to SEQ ID NO: 1, preferably IgA class antibody, for the manufacture of a diagnostic kit accordance with this aspect, the invention particularly relates to a use of a polypeptide comprising SEQ ID NO: 1 or a variant thereof for the manufacture of a diagnostic kit. Moreover, the invention also provides a use of an antibody to SEQ ID NO: 1, preferably IgA, and/or IgG, more preferably IgA class antibody, for the manufacture of a diagnostic kit.

The diagnostic kit is preferably for the diagnosis of a SARS-CoV-2 infection, or for the differential diagnosis of a coronavirus infection, preferably for distinguishing between a SARS-CoV, preferably SARS-CoV-2, MERS, NL63, 229E, 0043 and HKU1 infection. Accordingly, the invention relates to a use of a polypeptide comprising SEQ ID NO: 1 or a variant thereof for the manufacture of a diagnostic kit for the diagnosis of a SARS-CoV-2 infection. Said diagnosis of a SARS-CoV-2 infection may, for example, comprise a step of detecting the presence or absence of an antibody to SEQ ID NO: 1, preferably an IgA IgM and/or IgG, more preferably IgA class antibody to SEQ ID NO: 1, in a sample from a subject. Said diagnosis of a SARS-Cod/-2 infection may also comprise a step of obtaining a sample from a subject, and a step of detecting the presence or absence of an antibody to SEQ ID NO: 1, preferably an IgA, IgM and/or IgG, more preferably IgA class antibody to SEQ ID NO: 1, in the sample from the subject. The invention further relates to a use of a polypeptide comprising SEQ ID NO: 1 ora variant thereof for the manufacture of a diagnostic kit for the differential diagnosis of a coronavirus infection, preferably for distinguishing between a SARS-CoV, preferably SARS-Cod'-2, MERS, NL63, 229E, 0043 and HKU1 infection. Said differential diagnosis may, for example, comprise a step of detecting the presence or absence of an antibody to SEQ ID NO: 1 in a sample from a subject, preferably an IgA, IgM and/or IgG, more preferably IgA and/or IgG class antibody to SEQ ID NO: 1, most preferably an IgA class antibody to SEQ ID NO: 1. Said differential diagnosis may also comprise a step of obtaining a sample from a subject, and a step of detecting the presence or absence of an antibody to SEQ ID NO: 1 in the sample from the subject, preferably an IgA, IgM and/or IgG, more preferably IgA and/or IgG class antibody to SEQ ID NO: 1, most preferably an IgA class antibody class antibody to SEQ ID NO: 1.

In accordance with this aspect, the invention likewise provides a polypeptide comprising SEQ ID NO: 1 or a variant thereof for use in diagnosis, for example for use in a diagnostic method practiced on the human or animal body. In particular, the invention provides a polypeptide comprising SEQ ID NO: 1 or a variant thereof for use in the diagnosis of a SARS-CoV-2 infection. Said diagnosis of a SARS-CoV-2 infection may, for example, comprise a step of detecting the presence or absence of an antibody to SEQ ID NO: 1, preferably an IgA, IgM and/or IgG, more preferably IgA and/or IgG class antibody to SEQ ID NO: 1, most preferably an IgA class antibody to SEQ ID NO: 1, in a sample from a subject. Said diagnosis of a SARS-Cod/-2 infection may also comprise a step of obtaining a sample from a subject, and a step of detecting the presence or absence of an antibody to SEQ ID NO: 1, preferably an IgA, IgM and/or IgG, more preferably IgA and/or IgG class antibody to SEQ ID NO: 1, most preferably an IgA class antibody to SEQ ID NO: 1, in the sample from the subject. The invention further provides a polypeptide comprising SEQ ID NO: 1 or a variant thereof for use in the differential diagnosis of a coronavirus infection, preferably for distinguishing between a SARS-CoV (preferably SARS-CoV-2), MERS, NL63, 229E, 0043 and HKU1 infection. Said differential diagnosis may, for example, comprise a step of detecting the presence or absence of an antibody to SEQ ID NO: 1 in a sample from a subject, preferably an IgA, IgM and/or IgG, more preferably IgA and/or IgG class antibody to SEQ ID NO: 1, most preferably an IgA class antibody to SEQ ID NO: 1. Said differential diagnosis may also comprise a step of obtaining a sample from a subject, and a step of detecting the presence or absence of an antibody to SEQ ID NO: 1 in the sample from the subject, preferably an IgA, IgM and/or IgG, more preferably IgA and/or IgG class antibody to SEQ ID NO: 1, most preferably an IgA class antibody to SEQ ID NO: 1.

The invention further provides an antibody to SEQ ID NO: 1, preferably an IgA, IgM and/or IgG, more preferably IgA and/or IgG class antibody to SEQ ID NO: 1, most preferably an IgA class antibody, for use in diagnosis, for example for use in a diagnostic method practiced on the human or animal body. In particular, the invention relates to an antibody to SEQ ID NO: 1, preferably an IgA, IgM and/or IgG, more preferably IgA and/or IgG class antibody to SEQ ID NO: 1, most preferably an IgA class antibody, for use in the diagnosis of a SARS-CoV-2 infection. The invention further relates to an antibody to SEQ ID NO: 1, preferably an IgA, IgM and/or IgG, more preferably IgA and/or IgG class antibody to SEQ ID NO: 1, most preferably an IgA class antibody, for use in the differential diagnosis of a coronavirus infection, preferably for distinguishing between a SARS-CoV, preferably SARS-CoV-2, MERS, NL63, 229E, 0043 and HKU1 infection.

In a 6¹h aspect, the invention provides a use of a recombinant antibody binding to SEQ ID NO: 1 which is preferably recognized by a secondary antibody binding to IgA class antibodies as a calibrator for the early diagnosis of a SARS-CoV-2 infection.

In accordance with this aspect, the invention also provides a use of a recombinant antibody, for example a recombinant IgA class antibody, binding to SEQ ID NO: 1 as a calibrator, preferably as a calibrator for the detection of an antibody to SEQ ID NO: 1, preferably an IgA class antibody to SEQ ID NO: 1, in a sample from a subject.

Such a calibrator can be used, in particular, in a method of diagnosing a SARS-CoV-2 infection for in a method for the differential diagnosis of a coronavirus infection, preferably for distinguishing between a SARS-CoV, preferably SARS-CoV-2, MERS, NL63, 229E, 0043 and HKU1 infection. The invention thus likewise provides a use of a recombinant antibody, for example a recombinant IgA class antibody, binding to SEQ ID NO: 1 as a calibrator, preferably for the diagnosis, particularly for the early diagnosis, of a SARS-CoV-2 infection.

In a preferred embodiment, the human subject is a vaccinated human subject.

In a preferred embodiment, the method further comprises evaluating the result for the diagnosis. In a preferred embodiment, the method further comprises transferring the result of the diagnosis or the evaluation to a different location.

Various antigens and antibodies have been used as the basis for the immunological detection of coronaviruses, among them the whole virus or any of the structural proteins or fragments thereof as well as antibodies binding to these antigens.

The present invention is based on the inventors' surprising finding that antibodies against SEQ ID NO: 1 can be detected at an early stage of the infection. They may be detected for the purpose of determining the presence of an infection at an early stage of the infection.

In other words, the results disclosed herein demonstrate a superior sensitivity, preferably as measured by the number of correctly positive determined samples relative to the total number of samples examined, for diagnosing a SARS-CoV-2 infection already at early stage of infection. An important contribution to the observed sensitivity increase arises from the surprising finding that IgA class antibodies to SEQ IDNO:1 become detectable earlier than antibodies of other Ig classes, such as IgG antibodies in many patients, including a subpopulation of patients who lack detectable 10,1 antibodies.

Moreover, the invention is based on the surprising finding that antibodies to SEQ ID NO: 1, preferably IgG and/or IgA, persist longer and are detectable for a longer period of time than antibodies to SARS-CoV-2 N protein.

Also, there is surprisingly low cross reactivity with a range of antibodies in samples from patients infected with other coronaviruses, preferably other than SARS-CoV-1 and SARS-CoV-2, which contributes to the superior specificity, preferably as measured by the high number of correctly identified negative samples, i.e., low number of false-positive identified samples relative to the total number of samples examined, of detection of antibodies to SEQ ID NO: 1. In particular, the inventors have found that the specificity of SEQ ID NO: 1 as an antigen is superior compared to SEQ ID NO: 33 (S2 domain) (Okba et al., Severe Acute Respiratory Syndrome Coronavirus 2-Specific Antibody Responses in Coronavirus Disease Patients, Emerg Infect Dis. 2020 July; 26(7):1478-1488, prepublished on medRxiv 2020.03.18.20038059).

In a preferred embodiment, the term “diagnosis”, as used herein, is to be understood in its broadest possible sense and may refer to any kind of procedure aiming to obtain information instrumental in the assessment whether a patient suffered, suffers or is likely or more likely than the average or a comparative subject, the latter preferably having similar symptoms, to suffer from a certain disease or disorder in the past, at the time of the diagnosis or in the future, to find out how the disease is progressing or is likely to progress in the future or to evaluate the responsiveness of a patient or patients in general with regard to a treatment, preferably a vaccine, or to find out whether a sample is from such a patient. Such information may be used for a clinical diagnosis but may also be obtained by an experimental and/or research laboratory for the purpose of general research, for example to determine the proportion of subjects suffering from the disease in a patient cohort or in a population. In other words, the term “diagnosis” comprises not only diagnosing, but also prognosticating and/or monitoring the course of a disease or disorder, including monitoring the response of one or more patients to the administration of a drug or candidate drug, for example to determine its efficacy. Again, the early emergence and/or the long persistence of IgA and/or IgG antibodies to SEQ ID NO: 1 may be exploited. While the result may be assigned to a specific patient for clinical diagnostic applications and may be communicated to a medical doctor or institution treating said patient, for example by telephone, fax, letter or in an electronic format such as e-mail or using a data base, this is not necessarily the case for other applications, for example in diagnostics for research purposes, where it may be sufficient to assign the results to a sample from an anonymized patient or a patient cohort. In a preferred embodiment, the person to be diagnosed, i.e., the “subject” or “patient”, is an anonymous blood donor whose blood may be donated or used to obtain therapeutically useful antibodies. Preferably, the disease is a SARS infection, including SARS-CoV-1 and SARS-CoV-2, more preferably a SARS-CoV-2 infection,

In a preferred embodiment, the methods, products and uses according to the present invention may be used for interaction studies, including determining whether a drug candidate or other compound, including a candidate vaccine, or any bodily compound such as a blocking antibody is present and may interfere with the binding of an antibody to SARS-CoV-2 or may affect any downstream process, In a preferred embodiment, they may be used for monitoring the immune response, more preferably the emergence and/or titer of antibodies to a polypeptide comprising, preferably consisting of SEQ ID NO: 1, following the administration of an immunogenic composition comprising a polypeptide comprising SEQ ID NO: 1 or an immunogenic variant thereof, for example to a mammal, which may be a mammal other than a human, such as a laboratory animal. The detection of IgA and/or IgG antibodies, preferably in the late phase of the immunization of a subject, more preferably for the purpose of increasing the sensitivity of the detection or optimizing the sensitivity of the detection, is particularly preferred. In a preferred embodiment, the immunization is the result of a SARS-CoV-2 infection or the result of an administration of a vaccine comprising SEQ ID NO: 1 or a variant thereof. The increase or optimization may be in comparison to other antibodies against SARS-CoV-2 or components thereof, for example SEQ ID NO: 30 (N protein), preferably IgG class antibodies to SEQ ID NO: 30, In a preferred embodiment, the term “late phase”, as used herein, refers to the period beginning with day 20, 30, 40, 50, 60, 61, 70, 80 or 90 after the onset of the disease or the first administration of a vaccine, preferably day 60. Preferably it lasts for 28 days, 1, 2, 3, 4, 5, 6, 9, 12, 15, 18, 24, 36, 48, 60 or more months, preferably 3 or more months. In a preferred embodiment, a method or use may be for the long-term monitoring of an immunization. The immunization may be the result of a SARS-CoV-2 infection or of a vaccination, preferably with SEQ ID NO: 1 or a variant thereof. At least one sample obtained during the late phase is analyzed, preferably two or more. A sample may be obtained and analyzed at least once a week or once a months during the late phase.

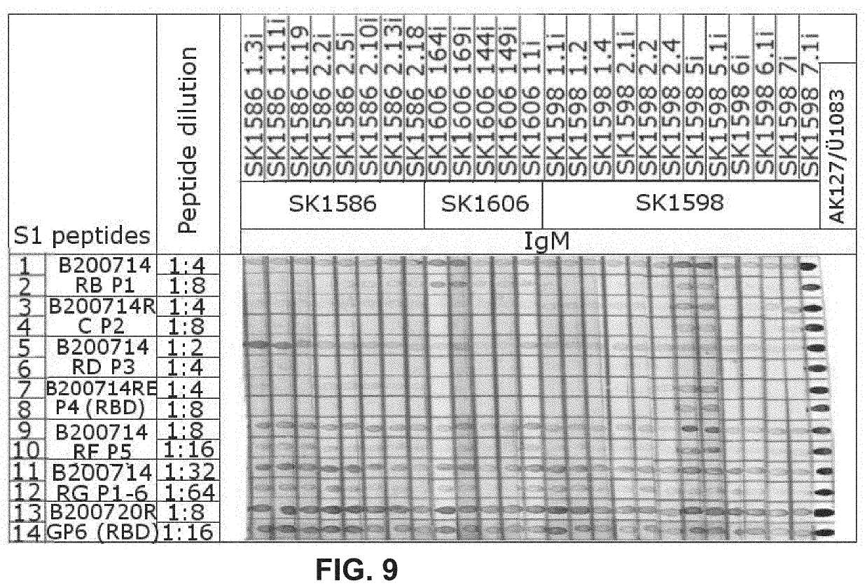

The subject is likely or more likely to suffer from a SARS-CoV-2 infection if an IgA and/or IgG and/or IgM antibody to SEQ ID NO: 1 is detected in a sample from them. The person skilled in the art is familiar with general principles in virology regarding the interpretation of results reflecting the presence or absence of antibodies (for example, Doerr, H. W., and Gerlich, W., Medizinische Virologie: Grundlagen, Diagnostik, Prävention and Therapie, Thieve 2010, for example FIG. 9.7 therein). Briefly, the first detection of specific antibodies to virus-specific antigens may be used for the initial diagnosis of an infection. IgA antibodies are usually not diagnostically relevant except for specific cases, such as in cases of infections by entero viruses (Gressner/Arndt, Lexikon der Medizinischen Labordiagnostik, 2. Auflage, Springer, 2013, page 1387), but if they appear, their titer will typically be lower than IgM and IgG class antibodies, and they may appear later. A low concentration of IgG class antibodies, preferably in the absence of IgM and IgA class antibodies, indicates an immunization in the past. An increasing IgG class antibody titer or high concentration may be indicative of an acute infection or reinfection. In a preferred embodiment, the presence of an antibody to SEQ ID NO: 1 indicates an immunization, either as a result of a previous or ongoing SARS-CoV-2 infection or a vaccination. In a preferred embodiment, a successful vaccination with a vaccine comprising a polypeptide comprising SEQ ID NO: 1 or a variant thereof (or with a nucleic acid encoding a polypeptide comprising SEQ ID NO: 1 or variant thereof, e.g. a RNA-based vaccine), may be distinguished from a SARS-CoV-2 infection by confirming the absence of antibodies to SARS-CoV-2 antigens other than SEQ ID NO: 1, preferably by confirming the absence of antibodies to SEQ ID NO: 30 (SARS-CoV-2 N protein).

The sample is preferably a mammalian sample, i.e., a sample from a mammal, more preferably a human sample, i.e., a sample from a human.

The term “diagnosis” does preferably not imply that the diagnostic methods or agents according to the present invention will be definitive and sufficient to finalize the diagnosis on the basis of a single test, let alone parameter, but may refer to a contribution to what is referred to as a “differential diagnosis”, i.e. a systematic diagnostic procedure considering the likelihood of a range of possible conditions on the basis of a range of diagnostic parameters. According to the invention, it can be distinguished if a patient, preferably one already suspected to have a coronavirus infection, suffers from a SARS, preferably SARS-CoV-1 and/or SARS-CoV-2 infection, or another coronavirus infection, preferably from the group comprising MERS, NL63, 229E, OC43 and HKU1. NL63, 229E, OC43 and HKU1 are associated with a significant number of cases of common cold, hence the differential diagnosis may involve distinguishing between SARS-CoV-1 and/or SARS-CoV-2 and a cold. For example, a patient may initially be suspected of suffering from a coronavirus infection owing to a possible exposure to a risk environment and/or based on common symptoms such as fever, cough and shortness of breath. A FOR (for example Corman V M, Landt O, Kaiser M, et al. Detection of 2019 novel coronavirus (2019-nCoV) by real-time RT-PCR, Euro Sumeill. 2020:25(3):2000045. doi:10.2807/1560-7917.ES.2020.25.3.2000045) and/or an immunoassay may then be carried out. If the FOR is negative, for example because the sample was taken a few days after the infection, the inventive method may be used to detect the presence or absence of an antibody to SEQ ID NO: 1. In addition, the presence or absence of an antibody to an antigen from another coronavirus may be detected, preferably from the group comprising SARS-CoV-1, MERS, NL63, 229E, 0043 and HKU1, more preferably MERS, NL63, 229E, 0043 and HKU1. Antibodies to homologues and variants of SEQ ID NO: 1 from SARS-CoV-2 may be detected. Based on specific clinical symptoms such as headache and body pains, which are more characteristic of SARS-CoV-1, or loss of taste and smell and sore throat, which are more characteristic for SARS-CoV-2, the diagnosis may be finalized. It may be considered whether the patient has been exposed to infected patients. For example, SARS-CoV-1 cases are extremely rare, so many patients suffering from common SARS-CoV-1 and SARS-CoV-2 symptoms in a SARS-CoV-2 pandemic can be assumed to suffer from an infection with the latter coronavirus. Distinction between SARS-CoV-1 and SARS-CoV-2 is possible based on the different time-resolved immunoglobulin (Ig) class signature, in particular the later emergence of IgA class antibodies in SARS-CoV-1 (Hsue, P. R., Huang, L. M., Chen, P. J., Kao, C. L., and Yang P. C. (2004) Chronological evolution of IgM, IgA, IgG and neutralization antibodies after infection with SARS-associated coronavirus, Clinical Microbiology and Infection, 10(12), 1062-1066). Antibody levels may be monitored, for example over several weeks, for example to detect the disappearance or emergence of an antibody of interest, which may help distinguish a primary and a secondary infection or immunization, for example as a result of vaccination, or recognize an infection with more than one coronavirus.

In a preferred embodiment, the term “SARS-CoV-2”, as used herein, refers to a virus characterized by the genome deposited on GenBank under accession code MN908947 or SEQ ID NO: 13, preferably as shown in SEQ ID NO: 13, and derivatives thereof having at least 80, preferably 85, preferably 88, preferably 90, preferably 91, preferably 92, preferably 93, preferably 94, preferably 95, preferably 96, preferably 97, preferably 98, preferably 99, preferably 99.5, preferably 99.8, preferably 99.9 or 99.99 percent sequence identity over the entire genome nucleotide sequence. All data base entries or product codes used herein correspond to the version online at the earliest priority or filing date of the application. For example, for the SARS-CoV-2 genome sequence deposited under accession code MN908947, the version MN908947.3 (published on January 17, 2020) was online at the earliest priority or filing date of the present application. The nucleotide sequence disclose in MN908947.3 is identical with SEQ ID NO: 13. More preferably, mutants such as those from the group comprising the U.K. variant B.1.1,7, the South African variant B.1.351, the Brazilian variant P.1 and the Mink Variant from Denmark are included.

In a preferred embodiment, the SARS-CoV-2 infection to be diagnosed is or may be associated with the U.K variant B.1.1.7 characterized by a spike protein having, with reference to SEQ ID NO: 1, one or more mutations, preferably all mutations from the group comprising deletion(s) in His 54 and/or Va155, Gln486Tyr, Ala555Asp, Asp599Gly and Pro666His. Preferably, a variant of SEQ ID NO: 1 having one or more mutations, preferably all from the group comprising deletion(s) in His 54 and/or Va155, Gln486Tyr, Ala555Asp, Asp599Gly and Pro666His, is used for reagents, methods and uses according to the present invention. A variant of SEQ ID NO: 1 comprising all these mutations is represented by SEQ ID NO: 51.

In a preferred embodiment, the SARS-CoV-2 infection to be diagnosed is or may be associated with the South African variant B.1.351 characterized by a spike protein having, with reference to SEQ ID NO: 1, one or more mutations, preferably all mutations from the group comprising Lys402Gln, Glu469Lys, Gln486Tyr and Asp599Gly. Preferably, a variant of SEQ ID NO: 1 having one or more mutations, preferably all from the group comprising Lys402Gln, Glu469Lys, Gln486Tyr and Asp599Gly, is used for reagents, methods and uses according to the present invention. A variant of SEQ ID NO: 1 comprising all these mutations is represented by SEQ ID NO: 52.

In a preferred embodiment, the SARS-CoV-2 infection to be diagnosed is or may be associated with the Brazilian variant P.1 characterized by a spike protein having, with reference to SEQ ID NO: 1, one or more mutations, preferably all mutations from the group comprising Glu469Lys and Gin486Tyr. Preferably, a variant of SEQ ID NO: 1 having one or more mutations, preferably all from the group comprising Glu469Lys and Gin486Tyr, is used for reagents, methods and uses according to the present invention. A variant of SEQ ID NO: 1 comprising all these mutations is represented by SEQ ID NO: 53.

In a preferred embodiment, the SARS-CoV-2 infection to be diagnosed is or may be associated with the Mink Variant from Denmark characterized by a spike protein having, with reference to SEQ ID NO: 1, one or more mutations, preferably all mutations from the group comprising deletion(s) in His 54 and/or Va155, Asp599Gly and Tyr438Phe. Preferably, a variant of SEQ ID NO: 1 having one or more mutations, preferably all from the group comprising deletion(s) in His 54 and/or Val55, Asp599GIy and Tyr438Phe, is used for reagents, methods and uses according to the present invention. A variant of SEQ ID NO: 1 comprising all these mutations is represented by SEQ ID NO: 54.

In a preferred embodiment, the term “diagnosis” means that the method or product or use may be used for aiding in the diagnosis of a disease or identifying a subject with a risk of suffering from a disease. The term “diagnosis” may also refer to a method or agent used to choose the most promising treatment regime for a patient. In other words, the method or agent may relate to selecting a treatment regimen for a subject.

In a preferred embodiment, the method according to the present invention comprises the step of providing the diagnostically useful carrier and a sample from a patient suspected of being infected, preferably a mammalian, more preferably a human patient. The carrier is coated with the polypeptide comprising SEQ ID NO: 1 or variant thereof. The carrier may then be contacted with the sample under conditions allowing for binding of any antibodies to the polypeptide comprising SEQ ID NO: 1 or variant thereof. The sample may then be removed and the carrier may be washed to remove any remaining sample. A secondary antibody or similar reagent or means binding to the antibody to be detected and carrying a detectable label may then be contacted with the carrier under conditions allowing formation of a complex between any bound antibody and the secondary antibody. The carrier may be washed then to remove non-bound secondary antibody. Finally, the presence of the antibody is detected by checking whether the secondary antibody may be detected.

In a preferred embodiment, the term “SARS-CoV-2 infection” or similar terms, as used herein, refers to an infection of a subject, preferably a human subject, with SARS-CoV-2, i.e., the presence of the virus, at least temporarily, in the body of said subject, more preferably together with detectable levels of the virus itself and/or one or more biomarker from the group comprising a SARS-CoV-2 polypeptide from the group comprising the structural proteins, in particular S and N, more preferably the S1 domain, and antibodies binding to them, and/or a nucleic acid from SARS-CoV-2, the latter detectable by FOR. The subject may have clinical symptoms, preferably one or more, more preferably all from the group comprising fever, tiredness, dry cough, nasal congestion, runny nose, and sore throat. More severe symptoms include breathing difficulties, chest pain or pressure and sudden confusion. The disease may lead to complications such as pneumonia, acute respiratory distress syndrome, sepsis, septic shock and kidney failure. However, many infected subjects have mild symptoms only and may not even be aware of their infection.

In a preferred embodiment, the method is used more than once to examine samples from the same patient, preferably on different days. For example, the presence or absence of antibodies may be detected on a daily basis over one or two weeks. In a preferred embodiment, at least 2, 3, 4, 5, 6, 7, 8, 9 or 10 samples are examined on different days. In a preferred embodiment, samples are taken at least over a period of at least 2, 3, 4, 5, 6, 7, 8, 9, 10, 11, 12, 13, 14, 15 or 16 weeks,

The methods, reagents and uses according to the present invention may also be used for screening the potency and the usefulness of an antiviral drug or a vaccine or vaccine candidate. In this regard, it is of note that the method of the invention has been validated in a vaccinated human, i.e. an antibody to the polypeptide comprising the amino acid sequence of SEQ ID NO: 1 could be detected in a human vaccinated with a composition comprising a polypeptide comprising SEQ ID NO: 1. They may be used as part of vaccination trials and studies aiming to confirm the quality of vaccines in human and other subjects including laboratory animals or to confirm that a subject's immune system responds to the administration of a vaccine. Preferably the vaccine is based on SEQ ID NO: 1 or a fragment thereof. The methods, reagents and uses may be used to initially diagnose a patient before the initial administration of a vaccine to check whether said patient is already immunized, for example as a result of a previous infection with SARS-CoV-2 or a previous vaccination. For example, the patient may be selected for inclusion in or exclusion from a study or trial and monitored over time. In particular already infected subjects may be excluded from testing a vaccine candidate. It can be confirmed whether a previous vaccination is still effective, preferably based on the detection of IgA and/or IgG class antibodies to SEQ ID NO: 1.

The methods and reagents according to the present invention may also be used for screening whether donated blood is contaminated with coronavirus or whether a blood donor produces antibodies to SARS-CoV-2, preferably SEQ ID NO: 1, which may be extracted from his donated blood, for example for therapeutic uses. Following the detection of the presence or absence of IgG, IgM and/or IgA class antibodies, preferably IgG and IgA, more preferably IgA, to SEQ ID NO: 1, an antibody to SARS-CoV-2, preferably to SEQ ID NO: 1 may be purified from the blood donor's blood, for example by affinity chromatography or by contacting the carrier according to the present invention with the donated blood under conditions that are compatible with the formation of a complex comprising the antibody to SEQ ID NO: 1 and the carrier, removal of any remaining donated blood and separation of the antibody from the carrier. In a preferred embodiment, donated blood is selected from the group comprising whole blood, plasma, and serum, and may contain an anti-dotting reagent. Also a compound selected from the group comprising citrate, phosphate, dextrose and adenine, preferably all of them, may be present.

The antibody to be detected binds preferably specifically to SEQ ID NO: 1. Specific binding preferably means that the binding reaction is stronger than a binding reaction characterized by a dissociation constant of 1×10⁻⁵ M, more preferably 1×10⁻⁷ M, more preferably 1×10⁻⁸ M, more preferably 1×10⁻⁹ M, more preferably 1×10⁻¹⁰ M, more preferably 1×10⁻¹¹ M, more preferably 1×10⁻¹² M, as determined by surface plasmon resonance using Biacore equipment at 25° C. in PBS buffer at pH 7.

The teachings of the present invention may not only be carried out using polypeptides having the exact sequences referred to in this application explicitly, such as SEQ ID NO: 1 for example by function, name, sequence or accession number, or 6.

In accordance with the invention, the term “variant”, as used herein, also refers to at least one fragment of the full length sequence referred to, or a polypeptide comprising said fragment, more specifically to one or more amino acid or nucleic acid sequences which are, relative to the full length sequence, truncated at one or both termini by one or more amino acids. Such a fragment comprises or encodes a (poly-) peptide having at least 10, 15, 25, 50, 75, 100, 150, 200, 250, 300, 400, 500 or 600 successive amino acids of the original sequence or a variant thereof. For example, fragments of SEQ ID NO: 1 include SEQ ID NO: 12 and SEQ ID NO: 31. Two or more copies of a fragment may be fused for increased sensitivity.

It is also understood that the term “variant”, as used herein, also embraces such polypeptides or fragments thereof comprising amino acid sequences, preferably a fragment comprising at least 25, more preferably 50, more preferably 200 successive amino acids, that are at least 40, 50, 60, 70, 75, 80, 85, 90, 92, 94, 95, 96, 97, 98 or 99% identical to the reference amino acid sequence referred to or the fragment thereof, wherein amino acids other than those essential for the biological activity, for example the ability to bind specifically to an antibody of interest, or maintain the fold or structure of the polypeptide may be deleted or substituted and/or one or more such essential amino acids may be replaced in a conservative manner and/or amino acids are added or deleted such that the biological activity of the polypeptide is at least partially preserved. For example, fragments of SEQ ID NO: 1 in comprise SEQ ID NO: 5, SEQ ID NO: 12, SEQ ID NOs: 14-29, SEQ ID NOs: 35-37 and SEQ ID NOs: 40-43. The state of the art comprises various methods that may be used to align two given nucleic acid or amino acid sequences and to calculate the degree of identity, see for example Arthur Lesk (2008), Introduction to bioinformatics, Oxford University Press, 2008, 3^{rd} edition. In a preferred embodiment, the ClustalW software (Larkin, M. A., Blackshields, G., Brown, N. P., Chenna, R., McGettigan, P. A., McWilliam, H., Valentin, F., Wallace, I. M., Wilm, A., Lopez, R., Thompson, J. D., Gibson, T. J., Higgins, D. G. (2007): Clustal W and Clustal X version 2.0. Bioinformatics, 23, 2947-.2948) is used applying default settings.

SARS-CoV-2-related publications on specific amino acid sequences such as Beal et al. may aid the skilled one in designing variants (Beal, J., Mitechell, T., Wyschogrod, W., Manthey, J, and Clore, A. (2020) Highly Distinguished Amino Acid Sequences of 2019-nCoV (Wuhan Coronavirus) dol: doi.org/10.1101/2020.01.31.929497), as well as publications relating to SARS-CoV, for example Hua et a/. (Hua, R. Zhou, Y., Wang, Y., Hua, Y and Tong, T, (2004) Identification of two antigenic epitopes on SARS-CoV spike protein, BBR 319, 929-935), wherein homologous epitopes may be found and SARS-CoV-2 epitopes be identified on account of their homology. For example, possible epitopes may be derived from SEQ ID NO: 5. Dahlke et al. present an epitope mapping based on a microarray comprising overlapping 15mer peptides derived from the S1 polypeptide (Dahlke, C., Heidepriem, J., Kobbe, R., Santer R., Koch, T., Fathi, A., Ly, M. L, Schmiedel, S., Seeberger, P. H., ID-UKE COVID-19 study group, Addo, M. M., and Loeffler, F. F, (2020) doi.org/10.1101/2020.04.14.20059733doi). More specifically, peptides comprising amino acid sequences SSVLHSTQDLFLPFF (SEQ ID NO: 14, which is 30-44 of SEQ ID NO: 1), TWFHAIHVSGTNGTKRFDNPV (SEQ ID NO: 15, which is 48-68), NVVIKVCEFQFCNDPFLGVYYHKNNKSWMESEFRVYSSANNCTFEYVSQPFLMDL EG (SEQ ID NO: 16, which is 110-166), DLPQGFSALEPLVDL (SEQ ID NO: 17, which is 200-214), LLALHRSYLTPGDSSSGVVIAGAAAY (SEQ ID NO: 18, which is 226-250), QPRTFLLKYNENGTITDAVDCALDP (SEQ ID NO: 19, which is 256-277), NATRFASVYAWNRKR (SEQ ID NO: 20, which is 328-342), TGKIADYNYKLPDDF (SEQ ID NO: 21, which is 399-414), YNYLYRLFRKSNLKP (SEQ ID NO: 22, which is 434-448), FGRDIADTTDAVRDPQTLEILDI (SEQ ID NO: 23, which is 550-572), SNQVAVLYQDVNCTE (SEQ ID NO: 24, which is 590-604), AGCLIGAEHVNNSYECDIP (SEQ ID NO: 25, which is 632-650) of S1 protein were identified as IgA-reactive epitopes. Peptides comprising amino acid sequences YNSASFSTFKCYGVS (SEQ ID NO: 26, which is 354-368), STGSNVFQTRAGCLI (SEQ ID NO: 27. which is 622-636) of S1 protein were identified as IgG-reactive epitopes. Peptides comprising amino acid sequences SSVLHSTQDLFLPFF (SEQ ID NO: 28, which is 30-44) and LLALHRSYLTPGDSSSGWTAGAAAY (SEQ ID NO: 29, which is 226-250) of S1 protein were identified as IgM-reactive epitopes. Moreover, the inventors have shown that peptides having sequences RTQLPPAYTNS (SEQ ID NO: 41), LTPGDSSSGWTAG (SEQ ID NO: 35), YQAGSTPCNGV (SEQ ID NO: 36) and YGFQPTNGVGYQ (SEQ ID NO: 37) are reactive with antibodies from SARS-CoV-2 patients. Many other publications can be used by the person skilled in the art as guidance when designing variants (Zhang et al. (2020) Mining of epitopes on spike protein of SARS-CoV-2 from COVID-19 patients, Cell Res 30, 702-704 (2020). doi.org/10.1038/s41422-020-0366-x; Poh, C. M., Carissimo, G., Wang, B. et al. Two linear epitopes on the SARS-CoV-2 spike protein that elicit neutralizing antibodies in COVID-19 patients. Nat Commun 11, 2806 (2020), doi.org/10.1038/s41467-020-16638-2; Wang et al. (2020) SARS-CoV-2 Proteome Microarray for Mapping COVID-19 Antibody Interactions at Amino Acid Resolution, ACS Cent. Sd. 2020, 6, 12, 2238-2249). In a preferred embodiment, a polypeptide is used which comprises SEQ ID NO: 1 or a variant thereof and is folded and, more preferably comprises at least 150, 200, 250, 300, 400, 500 or 600 successive amino acids of SEQ ID NO: 1.

In a preferred embodiment, variants may, in addition, comprise chemical modifications, for example labels such as isotopic labels or detectable labels or covalent modifications such as glycosylation, phosphorylation, acetylation, decarboxylation, citrullination, hydroxylation and the like. The person skilled in the art is familiar with methods for the modification of polypeptides. Moreover, variants may also be generated by way of fusion with other known polypeptides or variants thereof, for example artificial linkers, affinity tags, other antigens and the like. For example, SEQ ID NO: 3, SEQ ID NO: 32, SEQ ID NO: 34 and SEQ ID NO: 38 are fusion proteins according to the present invention.

According to the present invention, a medical or diagnostic device such as the diagnostically useful carrier may be prepared by expressing a recombinant variant of SEQ ID NO: 1 comprising an affinity tag, optionally with an artificial linker which may include a protease cleavage site, in a cell such as a eukaryotic (such as a CHO or HEK293 cell) or prokaryotic (such as an E. coil cell) cell, contacting the expressed variant with a ligand binding specifically to the affinity tag, which ligand is immobilized on a solid phase, washing the solid phase such that non-specifically bound material from the cell is removed and eluting the expressed variant from the solid phase, preferably by adding an excess of non-immobilized ligand. The variant may then be immobilized on the device. Optionally, the affinity tag may be removed by contacting the variant with a protease, preferably a protease recognizing the protease cleavage site, preferably before the immobilization. The affinity tag may be selected from the group of tags comprising His, 18A, ACP, aldehyde, Avi, BCCP, calmodulin-binding peptide (CBP), chitin binding domain (CBD), E-Tag, ELK16, FLAG, flash, poly-glutamate, poly-aspartate, GST, Green fluorescent protein, HA, maltose binding protein, myc, nus, NE, ProtA, ProtC, Tho1d4, S-Tag, SnoopTag, SpyTag, SofTag, Streptavidin, Strep-tag II, T7 Epitope Tag, TAP, TO, Thioredoxin, Ty, V5, VSV and Xpress Tag. Useful proteases include, but are not limited to TEV, Thrombin, Faktor Xa or Enteropeptidase. Suitable linkers comprising a protease cleavage site (e.g. between SEQ ID NO: 1 or variant thereof and the affinity tag) may be comprised in the variant, and be part of the coding sequence of commercially available expression vectors, for example pET vector series (Novagen) that may be used for expression of said variant.

The variant of the polypeptide has biological activity. In a preferred embodiment, such biological activity is the ability to bind to the respective antibody. In a preferred embodiment it comprises an epitope having the ability or it has itself the ability to bind to an antibody to SEQ ID NO: 1, preferably an IgA class antibody to SEQ ID NO: 1, preferably from a sample from a patient suffering from SARS-CoV-2, wherein more preferably the epitope comprises a sequence comprising at least 5, 6, 7 or 8 amino acid residues. More preferably, it does not bind specifically to homologues of SEQ ID NO: 1 from other coronaviruses, preferably from the group comprising MERS (SEQ ID NO: 6), NL63 (SEQ ID NO: 10), 229E (SEQ ID NO: 7), 0043 (SEQ ID NO: 8) and HKU1 (SEQ ID NO: 9), more preferably from the group comprising SARS-CoV-1 (SEQ ID NO: 11), MERS, NL63, 229E, OC43 and HKU1, wherein specific binding is preferably defined and determined as outlined above, using Biacore equipment.

The person skilled in the art is familiar how to make diagnostically useful reagents based on suitable amino acid sequences and is able to provide both linear peptides, for example by chemical synthesis, and polypeptides, for example by recombinant expression. In particular, the person skilled in the art is aware that both conformational and sequential epitopes exist, and that a suitable strategy for expression and purification must be chosen to obtain a diagnostically useful polypeptide which may be used to detect an antibody such as the antibody to SEQ ID NO: 1 (for example Reischl, U., Molecular Diagnosis of Infectious diseases, Humana Press, 1998, for example chapters 12, 15, 16, 18 and 19). The person skilled in the art is also familiar with ways to evaluate the usefulness of recombinant proteins for test systems (for example Reischl, U., Molecular Diagnosis of Infectious diseases, Humana Press, 1998, for example chapters 21-26).

The detection of the antibody or complex for the prognosis, diagnosis, methods or kit according to the present invention comprises the use of a method selected from the group comprising immunodiffusion techniques, immunoelectrophoretic techniques, light scattering immunoassays, agglutination techniques, labeled immunoassays such as those from the group comprising radiolabeled immunoassays, enzyme immunoassays such as colorimetric assays, chemiluminescence immunoassays and immunofluorescence techniques. In a preferred embodiment, the complex is detected using a method selected from the group comprising immunodiffusion techniques, immunoelectrophoretic techniques, light scattering immunoassays, agglutination techniques, labeled immunoassays from the group comprising radiolabeled immunoassays, chemiluminescence immunoassays, immunofluorescence techniques and immunoprecipitation techniques. The person skilled in the art is familiar with these methods, which are also described in the state of the art, for example in Zane, H. D. (2001): Immunology—Theoretical & Practical Concepts in Laboratory Medicine, W. B. Saunders Company, in particular in Chapter 14. Preferably the test format is an ELISA and a microtiter plate comprising wells is used as a diagnostically useful carrier.

Preferably a polypeptide comprising SEQ ID NO: 1 or a variant thereof is immobilized on a solid phase of the diagnostically useful carrier. It may be directly immobilized on the solid phase when contacted with the sample, but a competitive assay, a capture bridge assay, an immunometric assay, a class-specific second antibody on the solid phase, a class capture assay, direct or indirect, may also be used. The principle of each of these formats is detailed in The Immunoassay Handbook, 3^{rd} edition, edited by David Wild, Elsevier, 2005. More preferably, the solid phase is a test strip or a well of a microtiter plate for ELISA, most preferably a well of a microtiter plate for ELISA.

In a preferred embodiment, a competitive assay format is used, wherein the antibody to be detected competes with another antibody to SEQ ID NO: 1 or another ligand binding specifically to SEQ ID NO: 1. The ligand binding specifically to SEQ ID NO: 1 may be selected from the group comprising an aptamer or antibody binding to SEQ ID NO: 1 and the human ACE2 receptor (SEQ ID NO: 39) or a variant thereof, the natural binding partner of the SARS-Colt-2 spike protein. Such a method may comprise providing a polypeptide comprising SEQ ID NO: 1 or variant thereof and the ligand binding specifically to SEQ ID NO: 1, preferably from the group comprising an antibody, an aptamer and the ACE receptor or a variant thereof, If the antibody to be detected is present, it will interfere with the formation of the complex or partially or fully displace the ligand binding specifically to SEQ ID NO: 1, thus reducing the number of the complexes. Any complex comprising the antibody to be detected may then be detected by precipitating the complex, for example using an affinity ligand attached to the polypeptide comprising SEQ ID NO: 1 or to a molecule binding to the antibody to be detected such as a secondary antibody. Examples of affinity ligands include glutathione and biotin. A binding partner of the affinity ligand may be coated on a solid phase which binds to the affinity ligand such as GST or streptavidin, respectively. Any complex precipitated may then be detected, preferably by detecting a detectable label attached to the ligand binding specifically to the polypeptide comprising SEQ ID NO: 1 or attached to the polypeptide comprising SEQ ID NO: 1, respectively. A specific competitive test has been published by Tan et al. (2020) A SARS-Col/-2 surrogate virus neutralization test based on antibody-mediated blockage of ACE2-spike protein protein interaction, Nature Biotechnology 38 (1073-1078).

Alternatively, the complex may be formed on the surface of a carrier if the ligand binding specifically to SEQ ID NO: 1 or the polypeptide comprising SEQ ID NO: 1 or a variant is coated on said surface. Normally, the presence or absence of IgA, IgM and IgG class antibodies interfering with the complex formation will be detected using such a format. Specific IgA class antibodies may be detected by isolating the antibodies of the Ig class of interest before, preferably from the group comprising IgA, IgM and IgG class antibodies preferably Ig, for example using Protein A or Protein G or a secondary antibody. The person skilled in the art is familiar with the synthesis, selection and use of aptamers (Thiviyanathan V, Gorenstein D G, Aptarners and the next generation of diagnostic reagents. Proteornics Din Appl. 2012;6(11-12):563-573. doi:10.1002/prca.201200042) and antibodies (Hurt M, Frenzel A, Schirrmann T, Dübel S. Selection of recombinant antibodies from antibody gene libraries. Methods Mol Biol. 2014;1101:305-20; Hanack K, Messerschmidt K, Listek M. Antibodies and Selection of Monoclonal Antibodies. Adv Exp Med Biol. 2016;917:11-22; Harold F. Stills, in The Laboratory Rabbit, Guinea Pig, Hamster, and Other Rodents, 2012) and with generating and selecting antibodies binding to specific targets (Lottspeich/Engels, Bioanalytic, Chapter 6 and references therein, Springer 2012).

In another preferred embodiment, a complex comprising a first and a second polypeptide comprising SEQ ID NO: 1 or a variant thereof, such as RBD, and the antibody to be detected may be formed in a liquid phase if the antibody is present in the sample, since the antibody has two or more binding sites, each binding to a polypeptide comprising SEQ ID NO: 1. Any complex comprising the antibody to be detected may then be detected by precipitating the complex, for example using an affinity ligand attached to the first polypeptide such as glutathione or biotin and a binding partner coated on a solid phase which binds to the affinity ligand such as GST or streptavidin, respectively, which solid phase may for example be a bead. Any complex precipitated may then be detected, preferably by detecting a detectable label attached to the second polypeptide. Alternatively, the complex may be formed on the surface of a carrier if the first polypeptide comprising SEQ ID NO: 1 or a variant thereof is coated on said surface. Alternatively, the first and the second polypeptide may each be labeled with two different labels which are detectable only if they are in close proximity, for example when bridged by the antibody to be detected. Again, the presence or absence of IgA, IgM and IgG class antibodies will be detected unless the specific Ig class antibodies have been isolated before, preferably from the group comprising IgA, IgM and IgG class antibodies, preferably IgA, for example using Protein A or Protein G or a secondary antibody. Preferably, in an early phase of the infection, when IgG antibodies have not yet been produced, IgA antibodies will predominantly or only be detected.

Any polypeptide, for example a polypeptide comprising SEQ ID NO: 1 or a secondary antibody, may be provided in any form and at any degree of purification, from tissues, fluids or cells comprising said polypeptide in an endogenous form, more preferably cells overexpressing the polypeptide, crude or enriched lysates of such cells, to purified and/or isolated polypeptide which may be essentially pure. In a preferred embodiment, the polypeptide is a native polypeptide, wherein the term “native polypeptide”, as used herein, refers to a folded polypeptide, more preferably to a folded polypeptide purified from cells, more preferably from prokaryotic or eukaryotic, preferably mammalian cells. A glycosylated form of the polypeptide may be used. Secondary antibodies are preferably pure, for example following purification based on Protein A or G as an affinity ligand. In a preferred embodiment, any polypeptide used or provided according to the present invention is used or provided in a folded state, in the absence of significant concentrations of denaturing reagents such as thiol-containing compounds such as DTT.

In a preferred embodiment, the presence or absence of an IgA, IgM and/or IgG class antibody to SEQ ID NO: 1 is detected. In another preferred embodiment, the presence or absence of IgA is detected only. In another preferred embodiment, the presence or absence of IgM is detected only. In another preferred embodiment, the presence or absence of IgG is detected only.

In a preferred embodiment, the presence or absence of IgA and the presence or absence of IgG is detected. More preferably, a secondary antibody to IgA class antibodies and a secondary antibody to IgG class antibodies are used or provided for this detection. In another preferred embodiment, the presence or absence of IgM and the presence or absence of IgG is detected. More preferably, a secondary antibody to IgM class antibodies and a secondary antibody to IgG class antibodies are used or provided for this detection. In another preferred embodiment, the presence or absence of IgM and the presence or absence of IgA is detected. More preferably, a secondary antibody to IgM class antibodies and a secondary antibody to IgA class antibodies are used or provided for this detection.

In a preferred embodiment, the presence or absence of an antibody, for example IgA, IgM and/or IgG to SEQ ID NO: 1, may be detected using a polypeptide comprising SEQ ID NO: 1 or a variant thereof, but the polypeptide may comprise additional sequences, preferably artificial sequences for example linkers or binding epitopes. However, such additional sequences are chosen such that the ability of SEQ ID NO: 1 or a variant thereof to bind specifically to the antibody to be detected or the diagnostic reliability, in particular sensitivity and/or specificity, is not significantly altered, let alone abolished. For example, a domain binding to SEQ ID NO: 1 or a fragment thereof, thus masking an epitope, should not be fused to the polypeptide or be present. According to the present invention, a secondary antibody detecting IgA, IgM and/or IgG class immunoglobulins to a SARS-CoV-2 antigen, preferably from the group comprising an antigen comprising SEQ ID NO: 1 or a variant thereof, an antigen comprising SEQ ID NO: 30 or a variant thereof, an antigen comprising SEQ

ID NO: 31 or a variant thereof and an antigen comprising SEQ ID NO: 33 or a variant thereof, preferably all, may be detected or used for the diagnosis, preferably early diagnosis of a SARS-CoV-2 infection. An antigen selected from the group comprising an antigen comprising SEQ ID NO: 30 or a variant thereof, an antigen comprising SEQ ID NO: 31 or a variant thereof and an antigen comprising SEQ ID NO: 33 or a variant thereof, preferably all, may be coated on a diagnostically useful carrier, preferably spatially separated or in a mixture, and contacted with a sample for detecting an antibody binding specifically to the respective antigen or antigens. In a preferred embodiment, the detection of an antibody to SEQ ID NO: 1 in addition to the detection of an antibody to such a SARS-CoV-2 antigen other than SEQ ID NO: 1 may increase the overall sensitivity of the assay, in particular diagnosing a SARS-CoV-2 infection at an early stage or for the differential diagnosis at an early stage.

In a preferred embodiment, the products, methods and uses of the present invention are configured such that presence or absence of an antibody to SEQ ID NO: 1 can be distinguished from the presence of an antibody to another SARS-CoV-2 antigen or one or more other antigen, preferably from SARS-CoV-2 N protein, more preferably from the group comprising SARS-CoV-2 N protein, SARS-CoV-2 M protein and SARS-CoV-2 E protein, most preferably from the group comprising SARS-CoV-2 N protein, SARS-CoV-2 M protein SARS-Coll-2 E protein and SARS-CoV-2 S protein epitopes other than those present on SEQ ID NO: 1. In a more preferred embodiment, a polypeptide comprising SEQ ID NO: 1 or a variant thereof is spatially separated from such other SARS-CoV-2 antigens or other coronavirus antigens when used to detect the presence or absence of an antibody to SEQ ID NO: 1. For example, the polypeptide comprising SEQ ID NO: 1 or a variant may be pure and/or isolated on a carrier. For example, it could be on a blot or microtiter well or bead, spatially separate from other antigens.

In a preferred embodiment, the products, methods and uses of the present invention are configured such that presence or absence of an antibody to SEQ ID NO: 1 can be detected, without determining whether the antibody detected belongs to a certain immunoglobulin class, This is often referred to as detecting the “total antibodies” to one or more antigens such as SEQ ID NO: 1. Detecting total antibodies, including IgA and IgG, increases the sensitivity of the assay, since IgA class antibodies present at an early stage and IgA and IgG class antibodies to SEQ ID NO: 1 at a late phase are detected as part of the total antibodies to SEQ ID NO: 1. Preferably, an antibody to SEQ ID NO: 1 can be distinguished from an antibody to another SARS-CoV-2 antigen such as N protein (SEQ ID NO: 30) or S2 domain (SEQ ID NO: 32), more preferably because other SARS-CoV-2 antigens are absent or spatially separated or an antibody to such another SARS-CoV-2 produces a signal which can be distinguished from a signal produced by an antibody to SEQ ID NO: 1.

In a preferred embodiment, the products, methods and uses of the present invention are configured such that presence or absence of an antibody to SEQ ID NO: 1 can be detected, without determining whether the antibody detected binds to SEQ ID NO:

1 or to another SARS-CoV-2 antigen such as N protein or S2 protein. This may be accomplished by using a mixture of antigens comprising SEQ ID NO: 1 or a variant thereof in combination with N protein (SEQ ID NO: 30) or a variant thereof or S2 domain (SEQ ID NO: 32) or a variant thereof. In a more preferred embodiment, the whole spike protein comprising S1 and S2 domain (SEQ ID NO: 32) may be used, optionally in combination with N protein (SEQ ID NO: 30). Again, the sensitivity of such an assay is increased since antibodies to SEQ ID NO: 1 including IgA and IgG, increases the sensitivity of the assay, since IgA class antibodies present at an early stage and IgA and IaG class antibodies to SEQ ID NO: 1 at a late phase are detected as part of the total antibodies to SEQ ID NO: 1.

In a preferred embodiment, the products, methods and uses of the present invention are configured such that presence or absence of an antibody to SEQ ID NO: 1 can be detected, without determining whether the antibody detected belongs to a certain immunoglobulin class. This procedure is often referred to as detecting the “total antibodies” to one or more antigens such as SEQ ID NO: 1. Detecting total antibodies, including IgA and IgG, increases the sensitivity of the assay, since IgA class antibodies present at an early stage are detected as part of the antibodies to SEQ ID NO: 1.

In a preferred embodiment, the absence of an antibody to SEQ ID NO: 1 may be detected, using an isolated, pure and/or recombinant polypeptide comprising SEQ ID NO: 1 or a variant thereof.

In a preferred embodiment, the presence or absence of an antibody to SARS-CoV-2 N protein, defined by SEQ ID NO: 30, is determined in addition, preferably an IgA, IgG and/or IgM antibody, more preferably IgG or IgM, most preferably IgG. The carrier according to the invention may be coated with a polypeptide comprising the SARS-CoV-2 N protein defined by SEQ ID NO: 30 or a variant thereof for this purpose.

In a preferred embodiment, the presence or absence of an antibody to the receptor binding domain (RBD) of the SARS-CoV-2 51 domain defined by SEQ ID NO: 31 is detected. The carrier according to the invention may be coated with a polypeptide comprising SEQ ID NO: 31 ora variant thereof for this purpose.

In a preferred embodiment, the presence or absence of an antibody to the SARS-CoV-2 S2 domain defined by SEQ ID NO: 32 of the SARS-CoV-2 spike protein is detected. The carrier according to the invention may comprise a polypeptide comprising SEQ ID NO: 32 or a variant thereof for this purpose.

In a preferred embodiment, a secondary antibody is an antibody binding to all antibodies from an antibody or immunoglobulin class, preferably a human antibody class, preferably IgA and/or IgG and/or IgIVI antibodies, preferably IgA. Secondary antibodies typically recognize the constant domain of said class or are polyvalent, with binding sites to various epitopes across the sequence or 3D structure shared by antibodies of said Ig class. Secondary antibodies are typically from a mammal other than a human or from a bird, preferably from chicken, rabbit, mouse, rat, horse, pig, donkey, goat, cow, camel, llama, or non-human primate. A wide range of them is commercially available.

According to the present invention, the SARS-CoV-2 infection may be detected at increased sensitivity at an early stage, preferably 5 or fewer days after the onset of disease symptoms.

In a further preferred embodiment, the method the invention further comprises evaluating the result for a diagnosis, The evaluation in accordance with the present invention is carried out to decide whether a treatment for the individual tested is required. The evaluation of the result of the diagnosis may include involving a physician in order to select an appropriate treatment if the diagnosis was positive. Importantly_{;} the method of the invention excluding the evaluation may be carried out at one location such as a country and the evaluation of the results of the diagnosis may be carried out at a different location.

In a different preferred embodiment, the method of the invention further comprises transferring the result of the diagnosis or the evaluation to a different location. This preferred embodiment relates to cases where the method of the invention, optionally including the evaluation step_{;} are carried out in one location such as one country and the results of the diagnosis and evaluation, respectively, are transferred to a different location such as a different country. The transfer may be effected by any means available to the skilled person. This includes electronic transfer of the data as well as factual transfer of read material. According to this preferred embodiment, in particular the physician or clinic treating the patient in case of a positive result may take the appropriate steps for successful treatment.

The diagnostically useful carrier is preferably selected from the group comprising a glass slide, preferably for microscopy, a biochip, a microarray, a microtiter plate, a lateral flow device, a test strip, a membrane, e.g. a nitrocellulose membrane, preferably a line blot, a chromatography column and a bead, preferably a microtiter plate.

In a preferred embodiment, the diagnostically useful carrier is a line blot (Raoult, D., and Dasch, G. A. (1989), The line blot: an immunoassay for monoclonal and other antibodies. Its application to the serotyping of gram-negative bacteria, J. Immunol., Methods, 125 (1-2), 57-65; WO2013041540). In a preferred embodiment, the term “line blot”, as used herein, refers to a test strip; more preferably membrane-based; that has been coated with one or more means for specifically capturing an antibody, preferably each of these means is a polypeptide. If two or more means are used, they are preferably spatially separated on the carrier. Preferably_{;} the width of the bands is at least 30, more preferably 40, 50, 60, 70 or 80% the width of the test strip. The line blot may comprise one or more control bands for confirming that it has been contacted with sample sufficiently long and under adequate conditions, in particular in the presence of human serum, or with a secondary antibody, respectively. A line blot is preferably made from a nitrocellulose membrane.

In another preferred embodiment, the diagnostically useful carrier is a bead. Various beads for numerous applications are commercially available, mainly based on carbohydrate, for example sepharose or agarose, or plastic. They may contain active or activatable chemical groups such as a carboxyl or tosyl or ester group, which can be utilized for the immobilization of a means for specifically capturing an antibody. Preferably, the beads are beads having an average diameter of from 0.1 μm to 10 μm, from 0.5 μm to 8 μm, from 0.75 μm to 7 μm or from 1 μm to 6 μm. The beads can be coated with the means for specifically capturing an antibody directly or via affinity ligands, for example biotin or glutathione and streptavidin or GST, respectively. For example, the bead may be coated with biotin or glutathione and the antigen may be fused with streptavidin or glutathione-S-transferase or a variant thereof, respectively. Preferably, the bead is provided in the form of an aqueous suspension having a bead content of from 10 to 90%, preferably from 20 to 80%, preferably from 30 to 70%, more preferably from 40 to 60% (wlw). The person skilled in the art is familiar with such beads (Diamindis, E. P., Chriopoulus, T. K., Immunoassays, 1996, Academic Press), which are commercially available, for example Bio-Flex 000H beads MC10026-01 or 1 71-50601 1 from Bio-Rad,

In a particularly preferred embodiment, the beads are paramagnetic beads, which can be easily concentrated on a surface with the aid of a magnet. For this purpose, commercial paramagnetic beads usually contain a paramagnetic mineral, for example iron oxide. A multiplicity of suitable paramagnetic beads is commercially available. A bead may be labeled with a detectable label,

In a preferred embodiment, a paramagnetic bead is used and washed or incubated in buffer by applying a magnetic field to concentrate and immobilize the beads, following removal of the buffer present and addition of new buffer. The magnetic field may then be discontinued to make the suspension of the beads in the new buffer more efficient. A buffer may be any buffered solution used according to the present invention including a diluted patient sample, an incubation buffer or a buffer comprising a secondary antibody.

In a preferred embodiment, the antibody is detected using a chemiluminescent label. In a preferred embodiment, this is a chemiluminescent enzyme, preferably selected from the group comprising luciferase, peroxidase, alkaline phosphatase and g-galactosidase or a variant thereof, which may turn over a chemiluminescent substrate without being consumed itself (Kricka, L. J. (2003). Clinical applications of chemiluminescence. Analytica chirnica acta, 500(1): 279-286). In another preferred embodiment, the chemiluminescent label is a small organic compound having no enzymatic activity catalyzing a chemiluminescence reaction, which emits a chemiluminescence signal upon being degraded when contacted with a chemiluminescence substrate solution which comprises inorganic and/or non-enzymatic organic compounds that are required for emitting the signal. Preferably, the small organic compound having no enzymatic activity is selected from the group comprising acridinium esters (Weeks, I., Beheshti, I., McCapra, F., Campbell, A. K,, Woodhead, J. S. (1983) Acridinium esters as high specific activity labels in immunoassay. Clin Chem 29: 1474-1479) and luminol or a chemiluminescent derivative thereof such as isoluminol, Such small organic compounds may be coupled to the secondary antibody. In the case of lumina, the substrate solution comprises H202 at a high pH. In the case of an acridinium ester, a mixture of H202 and sodium hydroxide is frequently used. The small organic compound is consumed upon emission of the chemiluminescence signal. In a preferred embodiment the chemiluminescence of the chemiluminescent label is detected for 1 to 60 seconds, preferably for 2 to 20 seconds, more preferably 3 to 15 seconds following initiation of the chemiluminescent detection reaction, In another preferred embodiment the chemiluminescence of the chemiluminescent label is detected for at least 0.5, 1, 1.5, 2, 2.5 or 3 seconds. In another preferred embodiment, the carrier is a microtiter plate comprising at least 8 wells that may be used for ELISA. At least one of the wells is coated with the means for specifically capturing an antibody, either directly or indirectly, preferably a polypeptide comprising SEQ ID NO: 1 or a variant thereof. At least 3, preferably 4, more preferably 5 calibrators, at defined concentrations may be used to set up a calibration curve for semi-quantitative analysis. When the inventive method is carried out, the calibrators, which typically cover a range of concentrations covering the calibrating curve, may be processed and developed in parallel to the samples. A secondary antibody comprising a detectable label such as an enzymatically active label may be provided, for example a label having horse radish peroxidase activity or alkaline phosphatase activity or an enzyme capable of chemiluminescence.

In another preferred embodiment, the carrier is a microarray. In a preferred embodiment, the term “microarray”, as used herein, refers to a chip spotted with a variety of spatially separate antigens, preferably at least 20, preferably 30, 40, 50, 80 or 100. Preferably each antigen is a peptide comprising or consisting of 5 to 25, preferably 7 to 15 successive amino acids spanning a fragment of SEQ ID NO: 1, more preferably spanning the RBD (SEQ ID NO: 31). A secondary antibody comprising a label, preferably a fluorescent label, may be used for the detection. Preferably other antigens are spotted, more preferably from the group of polypeptides comprising SEQ ID NO: 30 (SARS-CoV-2 N protein) and SEQ ID NO: 33 (SARS-CoV-2 S2 protein).

In another preferred embodiment, a glass slide is used, which is on or part of a carrier for microscopic immunofluorescence analysis. A cell, preferably a eukaryotic cell such as a HEK293 cell is on the slide. It may be covered with a mounting buffer, Various compositions and methods are described in the state of the art, for example in “Mountants and Antifades”, published by Wright Cell Imaging Facility, Toronto Western Research Institute University Health Network, (de.scribd.com/document/47879592/Mountants-Antifades), Krenek et al. (1989) Comparison of antifading agents used in immunofluorescence, J. Immunol. Meth 117, 91-97 and Nairn et al. (1969) Microphotometry in Immunofluorescence, Clin. Exp. Immunol. 4, 697-705. The cell expresses, preferably overexpresses a polypeptide comprising SEQ ID NO: 1 or a variant thereof. The carrier may comprise a mock-transfected cell, which has been transfected with the same vector as the cell overexpressing a polypeptide comprising SEQ ID NO: 1 or a variant thereof, but without the nucleic acid encoding for the latter. Such mock-transfected cell may serve as a negative control. Another cell may comprise an additional coronavirus, preferably SARS, more preferably SARS-CoV-2 antigen, for example N protein, S2 protein or RBD to detect an antibody.

According to the present invention, immunofluorescence may be used to detect an antibody. The person skilled in the art is familiar with the method (Storch, W. B., Immunofluorescence in Clinical Immunology: A Primer and Atlas, Birkhäuser, 2000; Wesseling J G, Godeke G J, Schijns V E, Prevec L, Graham F L, Horzinek M C, Rottier P J. Mouse hepatitis virus spike and nucleocapsid proteins expressed by adenovirus vectors protect mice against a lethal infection. J Gen Virol. 1993 October; 74 (Pt 10):2061-9. doi: 10.1099/0022-1317-74-10-2061. PMID: 8409930.). Briefly, an antigen, preferably a polypeptide comprising SEQ ID NO: 1 or a variant thereof, is immobilized on a carrier which may be a cell expressing said antibody and contacted with a sample, followed by detection of the antibody to be detected by fluorescence, preferably using a means for detecting the antibody labeled with a fluorescent label. In a preferred embodiment, the cell is a eukaryotic cell overexpressing the polypeptide, such as a cell selected from the group comprising HEK, Hela, CHO and Jurkat cells and derivatives thereof. In a preferred embodiment, the cell is a recombinant cell overexpressing the polypeptide, which is preferably under the control of a heterologous strong promoter.

According to the present invention, a lateral flow device may be used to detect an antibody. The person skilled in the art is familiar with lateral flow devices for this purpose (Lateral Flow Immunoassay, edited by Raphael Wong, Harley Tse, 2009, Springer; Paper-based diagnostics: Current Status and Future applications, Kevin J. Land, Springer 2019). Briefly, a lateral flow assay may be based on a membrane such as a nitrocellulose membrane which comprises a polypeptide comprising SEQ ID NO: 1 ora variant thereof comprising a detectable label. If the membrane is contacted with a sample, an antibody to be detected will bind to the antigen. The resulting complex will move driven by capillary forces on the membrane and will be immobilized on a test line on the membrane comprising a means for detecting the antibody_{;} typically a secondary antibody binding to the immunoglobulin class or classes of the antibody or the antibodies to be detected such as IgG and/or IgA and/or IgM. Preferably nanoparticles or beads are used as labels, for example gold nanoparticles or latex beads.

According to the present invention, a polypeptide, preferably the polypeptide comprising SEQ ID NO: 1 or a variant thereof, may be a recombinant protein, wherein the term “recombinant”, as used herein, refers to a polypeptide produced using genetic engineering approaches at any stage of the production process, for example by fusing a nucleic acid encoding the polypeptide to a strong promoter for overexpression in cells or tissues or by engineering the sequence of the polypeptide itself. The person skilled in the art is familiar with methods for engineering nucleic acids and polypeptides encoded (for example, described in Green M. R. and Sambrook, J. (2012), Molecular Cloning—A Laboratory Manual, Fourth Edition, CSH or in Brown T. A. (1986), Gene Cloning—an introduction, Chapman & Hall) and for producing and purifying native or recombinant polypeptides (for example Handbooks “Strategies for Protein Purification”, “Antibody Purification”, published by GE Healthcare Life Sciences, and in Burgess, R. R., Deutscher, M. P. (2009): Guide to Protein Purification). In another preferred embodiment, the polypeptide is an isolated polypeptide, wherein the term “isolated” means that the polypeptide has been enriched compared to its state upon production using a biotechnological or synthetic approach and is preferably pure, i.e. at least 60, 70, 80, 90, 95 or 99 percent of the polypeptide in the respective liquid consists of said polypeptide as judged by SDS polyacrylamide gel electrophoresis followed by Coomassie blue staining and visual inspection. Preferably any polypeptide on a carrier used as a means to capture an antibody is pure.

In a preferred embodiment, a detectable label is used to detect an antibody according to the present invention, which is a label that may be used to distinguish a population of molecules from others using biophysical detection methods. It is preferably selected from the group comprising a fluorescent, a radioactive, a chemiluminescent label, a heavy metal such as gold label, a nanoparticle, a bead or an enzymatically active label, preferably one catalyzing a colorimetric reaction. In a preferred embodiment, a fluorescent label is selected from the group comprising Alexa dyes, FITC, TRITC and green fluorescent protein (GFP). Iodine-125 may be used as radioactive label. In a preferred embodiment, an enzymatically active label is selected from the group comprising horseradish peroxidase, glucose oxidase, beta galactosidase, alkaline phosphatase and luciferase. The person skilled in the art is able to choose suitable labels and to attach them to proteins, nucleic acids and other molecules (Hassanzadeh L, Chen S, Veedu R N. Radiolabeling of Nucleic Acid Aptamers for Highly Sensitive Disease-Specific Molecular Imaging. Pharmaceuticals (Basel). 2018; 11(4):106. Published 2018 Oct. 15. doi:10.3390/ph11040106 Hassanzadeh L, Chen S, Veedu R N. Radiolabeling of Nucleic Acid Aptamers for Highly Sensitive Disease-Specific Molecular Imaging. Pharmaceuticals (Basel). 2018; 11(4):106. Published 2018 Oct. 15. doi:10.3390/ph11040106, Bioconjugate Techniques, 3rd Edition (2013) by Greg T. Hermanson, Obermaier C, Griebel A, Westermeier R. Principles of protein labeling techniques. Methods Mol Biol. 2015; 1295: 1153-65), and a wide range of labeled molecules are commercially available.

According to the present invention, a means for detecting the presence of antibodies to SEQ ID NO: 1 is provided. In a preferred embodiment, a secondary antibody comprising a detectable label may be used to detect IgA and/or IgG and/or IgM, preferably IgA class antibodies to SEQ ID NO: 1, more preferably SEQ ID NO: 1 and another coronavirus antigen. A protein having peroxidase activity may be used as an enzymatically active label. Preferably the secondary antibody recognizes mammalian, more preferably human antibodies. If antibodies from one Ig class are to be detected, two secondary antibodies may be used, preferably one binding to IgA class antibodies and one binding to IgG class antibodies. The two secondary antibodies may be in a mixture or separate, preferably separate to allow separate detection of antibodies from different Ig classes such as IgA, IgM and IgG antibodies, preferably IgG and IgA for example to obtain information regarding the course of the disease, based on the fact that IgA class antibodies emerge earlier in many patients than IgG class antibodies. Alternatively, one secondary antibody binding to antibodies from more than one Ig class may be used, such as from the group comprising IgG, IgA and IgM, preferably one binding to IgG and IgA class antibodies. A secondary antibody may be a polyclonal or monoclonal antibody. In a preferred embodiment, the secondary antibody is from a mammal other than a human but binds to human antibodies of a certain Ig class, preferably IgA, IgG and/or IgM. The person skilled in the art is familiar with the production and use of secondary antibodies (Kalyuzhny, A., Immunohistochemistry, Essential Elements and Beyound, Springer, 2017, in particular chapter 4; Howard, G. C., and Bethell, D. R., Basic Methods in Antibody Production and Characterization, 2000; CRC press). A variety of secondary antibodies, optionally with labels such as a fluorescent label, is commercially available, for example FITC-labeled secondary antibodies Cat # H15101, Cat # 62-8411 Cat # A24459, horseradish peroxidase labeled secondary antibodies Cat # 31420, Cat # SA1-35467, Cat # SA1-35467, Cat # SA1-35467 and others from Thermo Fisher. Labeled fragments of secondary antibodies or aptamers may also be used. The person skilled in the art is familiar with the synthesis, selection and use of aptamers, which may also be used as means for detecting the presence of antibody to SEQ ID NO: 1, for example when binding specifically to the antibody or antibodies to be detected, preferably IgG and/or IgA and/or IgM antibodies (Thiviyanathan V, Gorenstein DG. Aptamers and the next generation of diagnostic reagents. Proteornics Clin Appl. 2012; 6(11-12):563-573. doi:10.1002/prca.201200042) and the generation of specific antibodies (Hurt M, Frenzel A, Schirrmann T, Dübel S. Selection of recombinant antibodies from antibody gene libraries. Methods Mol Biol. 2014; 1101:305-20; Hanack K, Messerschmidt K, Listek M. Antibodies and Selection of Monoclonal Antibodies, Adv Exp Med Biol. 2016;917;11-22; Harold F. Stills, in The Laboratory Rabbit, Guinea Pig, Hamster, and Other Rodents, 2012). Such aptamers may bind specifically to the constant region or epitopes in other parts of the antibody or antibodies to be detected.

In another preferred embodiment, a polypeptide comprising SEQ ID NO: 1 or a variant thereof, preferably the receptor binding domain (SEQ ID NO: 31), may be used as a means for detecting the presence of an antibody to SEQ ID NO: 1. More specifically, said polypeptide may be coated to a diagnostically useful carrier and may be used to capture any antibody to be detected. Since human antibodies have more than one binding site, only one antigen binding site of a captured antibody may be occupied. Subsequent addition of another polypeptide comprising SEQ ID NO: 1 or a variant thereof, which is not coated on the carrier, may lead to the occupation of another binding site by this newly added polypeptide. The complex comprising a coated polypeptide, the antibody to be detected and the other polypeptide may then be detected. More preferably, the other polypeptide carries a detectable label, and this is used to detect the complex. In this embodiment, all antibodies, more specifically IgA, IgM and IgG class antibodies may be detected.

In another preferred embodiment, a polypeptide comprising SEQ ID NO: 39 (ACE2) or a variant thereof binding to the receptor binding domain in SEQ ID NO: 1, optionally in combination with a polypeptide comprising SEQ ID NO: 1 or a variant thereof binding to the ACE2 receptor, may be used as a means for detecting the presence of an antibody to SEQ ID NO: 1. The competitive assay format is then used to detect its presence.

In another preferred embodiment, specific proteins binding to distinct immunoglobulin classes may be labeled with detectable labels and used as a means for detecting the presence of an antibody to SEQ ID NO: 1. For example, protein G, A and L are bacterial proteins which bind to IgG class antibodies, (L. Bjorck, G. Kronvall: Purification and some properties of streptococcal protein G, a novel IgG-binding reagent. In: Journal of Immunology. 133(2)11984.), whereas jacalin (Abeam, ThermoFisher) may be used for binding of IgA class antibodies (see e.g. Choe et al., Materials (Basel). 2016 Dec; 9(12): 994; Wilkinson & Neville Vet Immunol Immunopathol. 1988 March; 18(2):195-8).

In a preferred embodiment, a kit according to the present invention comprises a polypeptide comprising SEQ ID NO: 1 or a variant thereof, preferably coated to a diagnostically useful carrier, more preferably a microtiter plate, and one or more, preferably all reagents from the group comprising a calibrator, a positive control, a negative control, a washing buffer, a means for detecting the presence of an antibody to SEQ ID NO: 1, preferably an IgA, IgM and/or IgG class antibody, preferably a secondary antibody binding specifically antibodies, more preferably IgA, IgM and/or IgG class antibodies, wherein the secondary antibody may comprise a detectable label, a sample buffer, a detection solution, preferably a chromogen/substrate solution, a stop solution and a protective foil.

In a preferred embodiment, a calibrator is a reagent that binds to a polypeptide comprising SEQ ID NO: 1 or a variant thereof and is preferably recognized by secondary antibodies recognizing IgA, IV and/or IgG class antibodies. The calibrator may be an IgA antibody to SEQ ID NO: 1. Alternatively, the calibrator may be a chimeric antibody, preferably comprising the constant region or other regions or epitopes shared by distinct immunoglobulin classes, preferably IgA and/or IgG and/or IgM, and a variable region, in particular a binding site which is derived from an artificial antibody. The person skilled in the art is familiar with the design, production and use of such calibrators (Lütkecosmann S, Faupel T, Porstmann S, Porstmann T, Micheel B, Hanack K. A cross-reactive monoclonal antibody as universal detection antibody in autoantibody diagnostic assays. Olin Chim Acta. 2019 December; 499:87-92. doi: 10.1016/j.cca.2019.09.003. Epub 2019 Sep. 4. PMID: 31493374, Hackett J Jr, Hoff-Velk J, Golden A, Brashear J, Robinson J, Rapp M, Klass M, Ostrow D H, Mandecki W. Recombinant mouse-human chimeric antibodies as calibrators in immunoassays that measure antibodies to Toxoplasma gondii. J Olin Microbiol. 1998 May; 36(5):1277-84. doi:, WO2009081165A1). In a preferred embodiment, a positive control is a solution comprising a compound such as antibody to SEQ ID NO: 1, preferably from the croup comprising IgA, IgG and IgM class antibodies, more preferably IgA, from the sample of a patient suffering from SARS-CoV-2 at an amount that a positive result is obtained using the method according to the present invention. A negative control is a reagent that lacks such a compound and could comprise serum from a healthy person. A washing buffer may be used to wash the carrier such as a microtiter plate after the incubation to remove unspecific antibodies and could be PBS. The means for detecting the presence of an antibody could be a secondary antibody binding to the antibody class to be detected, preferably human IgA, IgM and/or IgG class antibodies, and is labeled with a detectable label, preferably with an enzyme, more preferably with an enzyme having peroxidase activity. The sample buffer may be used to dilute patient sample and may be PBS. The detection solution may yield a signal in the presence of the labeled secondary antibody and is preferably a color-developing solution and more preferably 3,3′,5,5′ tetramethylbenzidine/H₂O₂. The stop solution may be added to a reaction to stop the reaction of the detection solution and may comprise a strong acid, preferably 0.5 M sulphuric acid. The protective foil may be placed on top of the carrier such as a microtiter plate to avoid evaporation.

Any reagent used according to the invention may comprise a preservative, for example azide.

According to the present invention, a use of a polypeptide comprising SEQ ID NO: 1 or a variant thereof or an antibody to SEQ ID NO: 1, preferably an IgA class antibody, for the manufacture of a diagnostic kit is provided. In a preferred embodiment, the polypeptide or antibody is packaged as one of the components of such a kit. The polypeptide or antibody may be used to confirm the quality upon production of the kit. For example, the antibody may be used as a positive control to confirm the reactivity of a polypeptide comprising SEQ ID NO: 1 which is a component of the kit, either separate or coated on a diagnostically useful carrier. The polypeptide may be used to confirm the reactivity of an antibody binding specifically to SEQ ID NO: 1 which is part of the kit. The polypeptide may be used to coat a diagnostically useful carrier as part of the manufacture. Both the polypeptide and antibody may be used to determine the concentration of the antibody or polypeptide, respectively, in buffered solutions used to make the carrier and the kit or to check whether or not a cell is expressing a polypeptide comprising SEQ ID NO: 1.

According to the present invention, an antibody to SEQ ID NO: 1, preferably IgA class antibody, is used for diagnosing a SARS-CoV-2 infection or for the differential diagnosis according to the present invention. The use may relate to detecting the antibody itself or in combination with other antibodies to SEQ ID NO: 1 or antibodies to other SARS-CoV-2 antigens, thus increasing the overall sensitivity of the diagnostic assay. In a more preferred embodiment, an IgA class antibody is used to increase the sensitivity of a diagnostic assay, in particular at an early stage of the infection. This may be accomplished by detecting not only IgG, but also IgA class antibodies, optionally also IgM class antibodies to SEQ ID NO: 1 or IgA only. In a more preferred embodiment, an IgG class antibody is used to increase the sensitivity of a diagnostic assay, in particular over a period of time which sees a decline in the concentration of antibodies to other antigens, for example IgG class antibodies to the N protein of SARS-CoV-2 such as during the late phase of a SARS-CoV-2 infection. This may be accomplished by detecting not only IgG, but also IgA class antibodies, optionally also IgM class antibodies to SEQ ID NO: 1 or IgG only. In another preferred embodiment, the antibody may be used for the diagnosis by calibrating a device or assay using a calibrator comprising said antibody, preferably an IgA, IgM and/or IgG class antibody, preferably all. In a preferred embodiment, IgA and/or IgG antibody to SEQ ID NO: 1 is used to increase the sensitivity at a late phase of the immunization. The antibody may be used for the validation of an assay, for calibration, for confirming the quality of reagents or assay materials such as a diagnostically useful carrier or as a positive control.

According to the present invention, a polypeptide comprising SEQ ID NO:1 or a variant thereof or an antibody to SEQ ID NO: 1, preferably an IgA class antibody is used for the manufacture of a diagnostic kit, preferably for the diagnosis of SARS-CoV-2, wherein an IgA class antibody binding specifically to SEQ ID NO: 1 is detected.

A use of an antibody to SEQ ID NO: 1, preferably IgA, IgM and/or IgG, more preferably IgA class antibody, for increasing the sensitivity of the detection of a SARS-CoV-2 infection, preferably at an early stage of the infection, more preferably 5 or fewer days after the onset of disease symptoms. In another preferred embodiment, the use may be at a late phase of the infection. In a preferred embodiment, the presence of or absence IgA, IgM and IgG to SEQ ID NO: 1 is detected.

The present invention comprises a range of novel nucleic acid and polypeptide sequences, including in particular the sequences described in the following and/or in the sequence listing forming part of the present specification. It will be understood that in case of conflict between any sequence shown herein below and the corresponding sequence described in the sequence listing, the present invention specifically and individually relates to each one of the respective sequences, i.e., to the sequence described herein below and also to the sequence described in the sequence listing.

SEQ ID NO: 1 (SARS-CoV-2 S1 domain from S Protein)
VNLTTRTQLPPAYTNSFTRGVYYPDKVFRSSVLHSTQDLFLPFFSNVTWFHAIHVSG
TNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRI
SNCVADYSVLYNSASFSTFKCYGVSPTKLNDLCFTNVYADSFVIRGDEVRQIAPGQT
GKIADYNYKLPDDFTGCVIAWNSNNLDSKVGGNYNYLYRLFRKSNLKPFERDISTEI
YQAGSTPCNGVEGFNCYFPLQSYGFQPTNGVGYQPYRVVVLSFELLHAPATVCGP
KKSTNLVKNKCVNFNFNGLTGTGVLTESNKKFLPFQQFGRDIADTTDAVRDPQTLEI
LDITPCSFGGVSVITPGTNTSNQVAVLYQDVNCTEVPVAIHADQLTPTWRVYSTGSN
VFQTRAGCLIGAEHVNNSYECDIPIGAGICASYQTQTNSPRRAR
SEQ ID NO: 2 (SARS-CoV-2 S1 domain from S Protein,
C-terminally his-tagged, as expressed in cells for the
example)
MFVFLVLLPLVSSQCVNLTTRTQLPPAYTNSFTRGVYYPDKVFRSSVLHSTQDLFLP
FFSNVTWFHAIHVSGTNGTKRFDNPVLPFNDGVYFASTEKSNIIRGWIFGTTLDSKT
QSLLIVNNATNVVIKVCEFQFCNDPFLGVYYHKNNKSWMESEFRVYSSANNCTFEY
VSQPFLMDLEGKQGNFKNLREFVFKNIDGYFKIYSKHTPINLVRDLPQGFSALEPLV
DLPIGINITRFQTLLALHRSYLTPGDSSSGWTAGAAAYYVGYLQPRTFLLKYNENGTI
TDAVDCALDPLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCFTNVYADS
FVIRGDEVRQIAPGQTGKIADYNYKLPDDFTGCVIAWNSNNLDSKVGGNYNYLYRLF
RKSNLKPFERDISTEIYQAGSTPCNGVEGFNCYFPLQSYGFQPTNGVGYQPYRVVV
LSFELLHAPATVCGPKKSTNLVKNKCVNFNFNGLTGTGVLTESNKKFLPFQQFGRDI
ADTTDAVRDPQTLEILDITPCSFGGVSVITPGTNTSNQVAVLYQDVNCTEVPVAIHAD
QLTPTWRVYSTGSNVFQTRAGCLIGAEHVNNSYECDIPIGAGICASYQTQTNSPRR
ARLEHHHHHHHH
SEQ ID NO: 3 (SARS-CoV-2 S1 domain from S Protein,
C-terminally his-tagged, as after cleavage of signal
peptide, used in the examples)
VNLTTRTQLPPAYTNSFTRGVYYPDKVFRSSVLHSTQDLFLPFFSNVTWFHAIHVSG
TNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRI
SNCVADYSVLYNSASFSTFKCYGVSPTKLNDLCFTNVYADSFVIRGDEVRQIAPGQT
GKIADYNYKLPDDFTGCVIAWNSNNLDSKVGGNYNYLYRLFRKSNLKPFERDISTEI
YQAGSTPCNGVEGFNCYFPLQSYGFQPTNGVGYQPYRVVVLSFELLHAPATVCGP
KKSTNLVKNKCVNFNFNGLTGTGVLTESNKKFLPFQQFGRDIADTTDAVRDPQTLEI
LDITPCSFGGVSVITPGTNTSNQVAVLYQDVNCTEVPVAIHADQLTPTWRVYSTGSN
VFQTRAGCLIGAEHVNNSYECDIPIGAGICASYQTQTNSPRRARLEHHHHHHHH
SEQ ID NO: 4 (SARS-CoV-2 S1 domain from S Protein,
nucleotide sequence encoding SEQ ID NO: 2)
ATGTTCGTATTCCTTGTTCTGCTGCCTTTGGTTAGCAGTCAGTGTGTCAACCTGA
CAACTCGCACGCAACTGCCGCCAGCTTACACCAACTCTTTCACAAGAGGCGTCT
ACTACCCGGACAAAGTGTTTCGCTCATCAGTGCTGCACTCTACACAAGATTTGTT
TCTGCCATTCTTCTCTAACGTAACCTGGTTTCACGCGATTCATGTGTCTGGGACA
AATGGGACCAAGCGCTTCGACAACCCCGTGCTGCCATTCAATGACGGGGTGTA
TTTTGCCTCCACCGAGAAATCCAATATCATCCGAGGATGGATTTTCGGTACTACG
CTGGACTCTAAAACGCAGTCTCTCTTGATCGTTAATAACGCCACAAATGTTGTCA
TTAAGGTGTGCGAGTTTCAGTTCTGTAATGATCCCTTTCTGGGTGTGTATTACCA
CAAGAATAACAAGTCATGGATGGAAAGCGAGTTTCGCGTGTACTCAAGTGCCAA
TAACTGCACATTCGAGTATGTGTCCCAGCCTTTCCTGATGGATCTCGAAGGCAA
ACAGGGGAACTTCAAGAATCTGCGCGAGTTCGTGTTTAAGAACATCGACGGTTA
TTTCAAGATCTACAGCAAACATACACCCATTAACCTGGTCAGGGATCTCCCTCAG
GGATTCTCCGCCCTGGAACCCTTGGTGGACTTGCCCATTGGGATTAACATCACT
AGATTCCAGACCCTGCTGGCCCTTCACCGTTCCTATCTTACTCCTGGCGACAGT
AGCAGTGGATGGACCGCAGGAGCAGCCGCTTACTATGTAGGCTATCTGCAGCC
ACGGACCTTCCTCCTCAAGTACAATGAAAATGGTACCATAACTGATGCTGTGGA
CTGCGCTCTGGATCCACTCTCCGAAACTAAATGCACCCTTAAAAGCTTCACGGT
CGAAAAGGGAATCTACCAGACAAGTAACTTTCGGGTACAACCCACTGAGTCCAT
CGTGCGGTTTCCTAACATCACAAATCTCTGCCCCTTTGGTGAAGTGTTTAACGC
CACTAGGTTCGCTTCTGTTTATGCGTGGAATCGGAAGAGGATTTCCAATTGCGT
GGCAGACTACTCTGTCCTGTATAATAGCGCTAGCTTCAGCACCTTCAAATGTTAC
GGGGTAAGCCCAACTAAACTGAACGACCTCTGTTTTACCAACGTGTATGCCGAT
AGCTTTGTCATACGAGGAGATGAGGTTCGTCAGATTGCTCCTGGCCAAACGGG
GAAAATCGCAGACTACAACTACAAGCTTCCCGACGACTTCACAGGATGCGTGAT
CGCGTGGAACTCAAATAATCTGGATAGCAAGGTTGGTGGCAATTATAACTACCT
GTATCGACTGTTCAGGAAAAGCAACCTCAAACCCTTTGAGCGCGACATCAGCAC
CGAGATATACCAAGCCGGTTCAACACCTTGCAATGGGGTGGAAGGGTTTAACTG
CTATTTCCCACTTCAGAGCTATGGGTTTCAGCCAACCAATGGAGTCGGCTACCA
GCCCTATCGGGTGGTAGTCCTGTCCTTTGAGCTGTTGCATGCGCCTGCCACAGT
CTGTGGCCCTAAGAAGAGTACGAATCTGGTGAAGAACAAGTGCGTCAACTTCAA
TTTTAACGGCTTGACTGGAACAGGAGTTCTGACCGAGTCCAACAAGAAATTCCT
TCCTTTTCAGCAGTTTGGAAGGGATATAGCCGACACTACCGATGCCGTTCGGGA
TCCACAGACACTGGAGATTCTGGACATTACTCCGTGCTCATTTGGCGGTGTATC
TGTCATCACACCTGGGACCAATACCTCAAATCAGGTGGCTGTGCTCTACCAGGA
TGTGAATTGTACCGAAGTTCCAGTGGCAATTCATGCCGATCAACTGACTCCCAC
CTGGAGAGTGTACAGTACTGGCAGTAACGTGTTTCAGACAAGAGCTGGCTGTCT
CATAGGCGCAGAACACGTCAACAACAGCTATGAGTGTGACATTCCGATCGGCG
CAGGCATCTGTGCATCCTACCAGACGCAAACCAACTCTCCCAGAAGAGCCAGG
CTCGAGCACCACCATCACCATCACCATCACTAA
SEQ ID NO: 5 (possible SARS-CoV-2 S1 epitope)
NLKPFERDISTE
SEQ ID NO: 6 (S1[MERS_CoV])
YVDVGPDSVKSACIEVDIQQTFFDKTWPRPIDVSKADGIIYPQGRTYSNITITYQGLFP
YQGDHGDMYVYSAGHATGTTPQKLFVANYSQDVKQFANGFVVRIGAAANSTGTVII
SPSTSATIRKIYPAFMLGSSVGNFSDGKMGRFFNHTLVLLPDGCGTLLRAFYCILEP
RSGNHCPAGNSYTSFATYHTPATDCSDGNYNRNASLNSFKEYFNLRNCTFMYTYNI
TEDEILEWFGITQTAQGVHLFSSRYVDLYGGNMFQFATLPVYDTIKYYSIIPHSIRSIQ
SDRKAWAAFYVYKLQPLTFLLDFSVDGYIRRAIDCGFNDLSQLHCSYESFDVESGVY
SVSSFEAKPSGSVVEQAEGVECDFSPLLSGTPPQVYNFKRLVFTNCNYNLTKLLSLF
SVNDFTCSQISPAAIASNCYSSLILDYFSYPLSMKSDLSVSSAGPISQFNYKQSFSNP
TCLILATVPHNLTTITKPLKYSYINKCSRLLSDDRTEVPQLVNANQYSPCVSIVPSTVW
EDGDYYRKQLSPLEGGGWLVASGSTVAMTEQLQMGFGITVQYGTDTNSVCPKLEF
ANDTKIASQLGNCVEYSLYGVSGRGVFQNCTAVGVRQQRFVYDAYQNLVGYYSDD
GNYYCLRACVSVPVSVIYDKETKTHATLEGSVACEHISSTMSQYSRSTRSMLKRRD
STYGPLQTPVGCVLGLVNSSLFVEDCKLPLGQSLCALPDTPSTLTPRSVR
SEQ ID NO: 7 (S1[HCoV-229E])
CQTTNGLNTSYSVCNGCVGYSENVFAVESGGYIPSDFAFNNWFLLTNTSSVVDGV
VRSFQPLLLNCLWSVSGLRFTTGFVYFNGTGRGDCKGFSSDVLSDVIRYNLNFEEN
LRRGTILFKTSYGVVVFYCTNNTLVSGDAHIPFGTVLGNFYCFVNTTIGNETTSAFVG
ALPKTVREFVISRTGHFYINGYRYFTLGNVEAVNFNVTTAETTDFCTVALASYADVLV
NVSQTSIANIIYCNSVINRLRCDQLSFDVPDGFYSTSPIQSVELPVSIVSLPVYHKHTFI
VLYVDFKPQSGGGKCFNCYPAGVNITLANFNETKGPLCVDTSHFTTKYVAVYANVG
RWSASINTGNCPFSFGKVNNFVKFGSVCFSLKDIPGGCAMPIVANWAYSKYYTIGSL
YVSWSDGDGITGVPQPVEGVSSFMNVTLDKCTKYNIYDVSGVGVIRVSNDTFLNGIT
YTSTSGNLLGFKDVTKGTIYSITPCNPPDQLVVYQQAVVGAMLSENFTSYGFSNVVE
LPKFFYA
SEQ ID NO: 8 (S1[HCoV-OC43])
AVIGDLKCTSDNINDKDTGPPPISTDTVDVTNGLGTYYVLDRVYLNTTLFLNGYYPTS
GSTYRNMALKGSVLLSRLWFKPPFLSDFINGIFAKVKNTKVIKDRVMYSEFPAITIGS
TFVNTSYSVVVQPRTINSTQDGDNKLQGLLEVSVCQYNMCEYPQTICHPNLGNHRK
ELWHLDTGVVSCLYKRNFTYDVNADYLYFHFYQEGGTFYAYFTDTGVVTKFLFNVY
LGMALSHYYVMPLTCNSKLTLEYWVTPLTSRQYLLAFNQDGIIFNAEDCMSDFMSEI
KCKTQSIAPPTGVYELNGYTVQPIADVYRRKPNLPNCNIEAWLNDKSVPSPLNWER
KTFSNCNFNMSSLMSFIQADSFTCNNIDAAKIYGMCFSSITIDKFAIPNGRKVDLQLG
NLGYLQSFNYRIDTTATSCQLYYNLPAANVSVSRFNPSTWNKRFGFIEDSVFKPRPA
GVLTNHDVVYAQHCFKAPKNFCPCKLNGSCVGSGPGKNNGIGTCPAGTNYLTCDN
LCTPDPITFTGTYKCPQTKSLVGIGEHCSGLAVKSDYCGGNSCTCRPQAFLGWSAD
SCLQGDKCNIFANFILHDVNSGLTCSTDLQKANTDIILGVCVNYDLYGILGQGIFVEVN
ATYYNSWQNLLYDSNGNLYGFRDYIINRTFMIRSCYSGRVSAAFHANSSEPALLFRN
IKCNYVFNNSLTRQLQPINYFDSYLGCVVNAYNSTAISVQTCDLTVGSGYCVDYSKN
RRSRG
SEQ ID NO: 9 (S1[HCoV-HKU1])
AVIGDFNCTNSFINDYNKTIPRISEDVVDVSLGLGTYYVLNRVYLNTTLLFTGYFPKS
GANFRDLALKGSIYLSTLWYKPPFLSDFNNGIFSKVKNTKLYVNNTLYSEFSTIVIGSV
FVNTSYTIVVQPHNGILEITACQYTMCEYPHTVCKSKGSIRNESWHIDSSEPLCLFKK
NFTYNVSADWLYFHFYQERGVFYAYYADVGMPTTFLFSLYLGTILSHYYVMPLTCN
AISSNTDNETLEYWVTPLSRRQYLLNFDEHGVITNAVDCSSSFLSEIQCKTQSFAPN
TGVYDLSGFTVKPVATVYRRIPNLPDCDIDNWLNNVSVPSPLNWERRIFSNCNFNLS
TLLRLVHVDSFSCNNLDKSKIFGSCFNSITVDKFAIPNRRRDDLQLGSSGFLQSSNY
KIDISSSSCQLYYSLPLVNVTINNFNPSSWNRRYGFGSFNLSSYDVVYSDHCFSVNS
DFCPCADPSVVNSCAKSKPPSAICPAGTKYRHCDLDTTLYVKNWCRCSCLPDPIST
YSPNTCPQKKVVVGIGEHCPGLGINEEKCGTQLNHSSCFCSPDAFLGWSFDSCISN
NRCNIFSNFIFNGINSGTTCSNDLLYSNTEISTGVCVNYDLYGITGQGIFKEVSAAYYN
NWQNLLYDSNGNIIGFKDFLTNKTYTILPCYSGRVSAAFYQNSSSPALLYRNLKCSY
VLNNISFISQPFYFDSYLGCVLNAVNLTSYSVSSCDLRMGSGFCIDYALPSSRRKRR
SEQ ID NO: 10 (S1[HCoV-NL63])
FFTCNSNANLSMLQLGVPDNSSTIVTGLLPTHWFCANQSTSVYSANGFFYIDVGNH
RSAFALHTGYYDANQYYIYVTNEIGLNASVTLKICKFSRNTTFDFLSNASSSFDCIVNL
LFTEQLGAPLGITISGETVRLHLYNVTRTFYVPAAYKLTKLSVKCYFNYSCVFSVVNA
TVTVNVTTHNGRVVNYTVCDDCNGYTDNIFSVQQDGRIPNGFPFNNWFLLTNGSTL
VDGVSRLYQPLRLTCLWPVPGLKSSTGFVYFNATGSDVNCNGYQHNSVVDVMRY
NLNFSANSLDNLKSGVIVFKTLQYDVLFYCSNSSSGVLDTTIPFGPSSQPYYCFINSTI
NTTHVSTFVGILPPTVREIVVARTGQFYINGFKYFDLGFIEAVNFNVTTASATDFWTV
AFATFVDVLVNVSATNIQNLLYCDSPFEKLQCEHLQFGLQDGFYSANFLDDNVLPET
YVALPIYYQHTDINFTATASFGGSCYVCKPHQVNISLNGNTSVCVRTSHFSIRYIYNR
VKSGSPGDSSWHIYLKSGTCPFSFSKLNNFQKFKTICFSTVEVPGSCNFPLEATWH
YTSYTIVGALYVTWSEGNSITGVPYPVSGIREFSNLVLNNCTKYNIYDYVGTGIIRSSN
QSLAGGITYVSNSGNLLGFKNVSTGNIFIVTPCNQPDQVAVYQQSIIGAMTAVNESR
YGLQNLLQLPNFYYV
SEQ ID NO: 11 (S1[SARS_CoV])
SGSDLDRCTTFDDVQAPNYTQHTSSMRGVYYPDEIFRSDTLYLTQDLFLPFYSNVT
GFHTINHTFGNPVIPFKDGIYFAATEKSNVVRGWVFGSTMNNKSQSVIIINNSTNVVI
RACNFELCDNPFFAVSKPMGTQTHTMIFDNAFNCTFEYISDAFSLDVSEKSGNFKHL
REFVFKNKDGFLYVYKGYQPIDVVRDLPSGFNTLKPIFKLPLGINITNFRAILTAFSPA
QDIWGTSAAAYFVGYLKPTTFMLKYDENGTITDAVDCSQNPLAELKCSVKSFEIDKG
IYQTSNFRVVPSGDVVRFPNITNLCPFGEVFNATKFPSVYAWERKKISNCVADYSVL
YNSTFFSTFKCYGVSATKLNDLCFSNVYADSFVVKGDDVRQIAPGQTGVIADYNYKL
PDDFMGCVLAWNTRNIDATSTGNYNYKYRYLRHGKLRPFERDISNVPFSPDGKPCT
PPALNCYWPLNDYGFYTTTGIGYQPYRVVVLSFELLNAPATVCGPKLSTDLIKNQCV
NFNFNGLTGTGVLTPSSKRFQPFQQFGRDVSDFTDSVRDPKTSEILDISPCSFGGV
SVITPGTNASSEVAVLYQDVNCTDVSTAIHADQLTPAWRIYSTGNNVFQTQAGCLIG
AEHVDTSYECDIPIGAGICASYHTVSLLRL
SEQ ID NO: 12 (fragment of SEQ ID NO: 1)
NSFTRGVYYPDKVFRSSVLHSTQDLFLPFFSNVTWFHAIHVSGTNGTKRFDNPVLP
FNDGVYFASTEKSNIIRGWIFGTTLDSKTQSLLIVNNATNVVIKVCEFQFCNDPFLGV
YYHKNNKSWMESEFRVYSSANNCTFEYVSQPFLMDLEGKQGNFKNLREFVFKNID
GYFKIYSKHTPINLVRDLPQGFSALEPLVDLPIGINITRFQTLLALHRSYLTPGDSSSG
WTAGAAAYYVGYLQPRTFLLKYNENGTITDAVDCALDPLSETKCTLKSFTVEKGIYQ
TSNFRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAGSTPCNGVE
GFNCYFPLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDITPCSFGGVS
VITPGTNTSNQVAVLYQDVNCTEVPVAIHADQLTPTWRVYSTGSNVFQTRAGCLIGA
EHVNNSYECDIPIGAGICASYQTQT
SEQ ID NO: 13 (genome of SARS-CoV-2 isolate Wuhan-Hu-1
genome, identical to Genbank MN908947):
ATTAAAGGTTTATACCTTCCCAGGTAACAAACCAACCAACTTTCGATCTCTTGTA
GATCTGTTCTCTAAACGAACTTTAAAATCTGTGTGGCTGTCACTCGGCTGCATGC
TTAGTGCACTCACGCAGTATAATTAATAACTAATTACTGTCGTTGACAGGACACG
AGTAACTCGTCTATCTTCTGCAGGCTGCTTACGGTTTCGTCCGTGTTGCAGCCG
ATCATCAGCACATCTAGGTTTCGTCCGGGTGTGACCGAAAGGTAAGATGGAGAG
CCTTGTCCCTGGTTTCAACGAGAAAACACACGTCCAACTCAGTTTGCCTGTTTTA
CAGGTTCGCGACGTGCTCGTACGTGGCTTTGGAGACTCCGTGGAGGAGGTCTT
ATCAGAGGCACGTCAACATCTTAAAGATGGCACTTGTGGCTTAGTAGAAGTTGA
AAAAGGCGTTTTGCCTCAACTTGAACAGCCCTATGTGTTCATCAAACGTTCGGAT
GCTCGAACTGCACCTCATGGTCATGTTATGGTTGAGCTGGTAGCAGAACTCGAA
GGCATTCAGTACGGTCGTAGTGGTGAGACACTTGGTGTCCTTGTCCCTCATGTG
GGCGAAATACCAGTGGCTTACCGCAAGGTTCTTCTTCGTAAGAACGGTAATAAA
GGAGCTGGTGGCCATAGTTACGGCGCCGATCTAAAGTCATTTGACTTAGGCGA
CGAGCTTGGCACTGATCCTTATGAAGATTTTCAAGAAAACTGGAACACTAAACAT
AGCAGTGGTGTTACCCGTGAACTCATGCGTGAGCTTAACGGAGGGGCATACAC
TCGCTATGTCGATAACAACTTCTGTGGCCCTGATGGCTACCCTCTTGAGTGCAT
TAAAGACCTTCTAGCACGTGCTGGTAAAGCTTCATGCACTTTGTCCGAACAACT
GGACTTTATTGACACTAAGAGGGGTGTATACTGCTGCCGTGAACATGAGCATGA
AATTGCTTGGTACACGGAACGTTCTGAAAAGAGCTATGAATTGCAGACACCTTTT
GAAATTAAATTGGCAAAGAAATTTGACACCTTCAATGGGGAATGTCCAAATTTTG
TATTTCCCTTAAATTCCATAATCAAGACTATTCAACCAAGGGTTGAAAAGAAAAA
GCTTGATGGCTTTATGGGTAGAATTCGATCTGTCTATCCAGTTGCGTCACCAAAT
GAATGCAACCAAATGTGCCTTTCAACTCTCATGAAGTGTGATCATTGTGGTGAAA
CTTCATGGCAGACGGGCGATTTTGTTAAAGCCACTTGCGAATTTTGTGGCACTG
AGAATTTGACTAAAGAAGGTGCCACTACTTGTGGTTACTTACCCCAAAATGCTGT
TGTTAAAATTTATTGTCCAGCATGTCACAATTCAGAAGTAGGACCTGAGCATAGT
CTTGCCGAATACCATAATGAATCTGGCTTGAAAACCATTCTTCGTAAGGGTGGTC
GCACTATTGCCTTTGGAGGCTGTGTGTTCTCTTATGTTGGTTGCCATAACAAGTG
TGCCTATTGGGTTCCACGTGCTAGCGCTAACATAGGTTGTAACCATACAGGTGT
TGTTGGAGAAGGTTCCGAAGGTCTTAATGACAACCTTCTTGAAATACTCCAAAAA
GAGAAAGTCAACATCAATATTGTTGGTGACTTTAAACTTAATGAAGAGATCGCCA
TTATTTTGGCATCTTTTTCTGCTTCCACAAGTGCTTTTGTGGAAACTGTGAAAGG
TTTGGATTATAAAGCATTCAAACAAATTGTTGAATCCTGTGGTAATTTTAAAGTTA
CAAAAGGAAAAGCTAAAAAAGGTGCCTGGAATATTGGTGAACAGAAATCAATAC
TGAGTCCTCTTTATGCATTTGCATCAGAGGCTGCTCGTGTTGTACGATCAATTTT
CTCCCGCACTCTTGAAACTGCTCAAAATTCTGTGCGTGTTTTACAGAAGGCCGC
TATAACAATACTAGATGGAATTTCACAGTATTCACTGAGACTCATTGATGCTATG
ATGTTCACATCTGATTTGGCTACTAACAATCTAGTTGTAATGGCCTACATTACAG
GTGGTGTTGTTCAGTTGACTTCGCAGTGGCTAACTAACATCTTTGGCACTGTTTA
TGAAAAACTCAAACCCGTCCTTGATTGGCTTGAAGAGAAGTTTAAGGAAGGTGT
AGAGTTTCTTAGAGACGGTTGGGAAATTGTTAAATTTATCTCAACCTGTGCTTGT
GAAATTGTCGGTGGACAAATTGTCACCTGTGCAAAGGAAATTAAGGAGAGTGTT
CAGACATTCTTTAAGCTTGTAAATAAATTTTTGGCTTTGTGTGCTGACTCTATCAT
TATTGGTGGAGCTAAACTTAAAGCCTTGAATTTAGGTGAAACATTTGTCACGCAC
TCAAAGGGATTGTACAGAAAGTGTGTTAAATCCAGAGAAGAAACTGGCCTACTC
ATGCCTCTAAAAGCCCCAAAAGAAATTATCTTCTTAGAGGGAGAAACACTTCCCA
CAGAAGTGTTAACAGAGGAAGTTGTCTTGAAAACTGGTGATTTACAACCATTAGA
ACAACCTACTAGTGAAGCTGTTGAAGCTCCATTGGTTGGTACACCAGTTTGTATT
AACGGGCTTATGTTGCTCGAAATCAAAGACACAGAAAAGTACTGTGCCCTTGCA
CCTAATATGATGGTAACAAACAATACCTTCACACTCAAAGGCGGTGCACCAACA
AAGGTTACTTTTGGTGATGACACTGTGATAGAAGTGCAAGGTTACAAGAGTGTG
AATATCACTTTTGAACTTGATGAAAGGATTGATAAAGTACTTAATGAGAAGTGCT
CTGCCTATACAGTTGAACTCGGTACAGAAGTAAATGAGTTCGCCTGTGTTGTGG
CAGATGCTGTCATAAAAACTTTGCAACCAGTATCTGAATTACTTACACCACTGGG
CATTGATTTAGATGAGTGGAGTATGGCTACATACTACTTATTTGATGAGTCTGGT
GAGTTTAAATTGGCTTCACATATGTATTGTTCTTTCTACCCTCCAGATGAGGATG
AAGAAGAAGGTGATTGTGAAGAAGAAGAGTTTGAGCCATCAACTCAATATGAGT
ATGGTACTGAAGATGATTACCAAGGTAAACCTTTGGAATTTGGTGCCACTTCTGC
TGCTCTTCAACCTGAAGAAGAGCAAGAAGAAGATTGGTTAGATGATGATAGTCA
ACAAACTGTTGGTCAACAAGACGGCAGTGAGGACAATCAGACAACTACTATTCA
AACAATTGTTGAGGTTCAACCTCAATTAGAGATGGAACTTACACCAGTTGTTCAG
ACTATTGAAGTGAATAGTTTTAGTGGTTATTTAAAACTTACTGACAATGTATACAT
TAAAAATGCAGACATTGTGGAAGAAGCTAAAAAGGTAAAACCAACAGTGGTTGTT
AATGCAGCCAATGTTTACCTTAAACATGGAGGAGGTGTTGCAGGAGCCTTAAAT
AAGGCTACTAACAATGCCATGCAAGTTGAATCTGATGATTACATAGCTACTAATG
GACCACTTAAAGTGGGTGGTAGTTGTGTTTTAAGCGGACACAATCTTGCTAAAC
ACTGTCTTCATGTTGTCGGCCCAAATGTTAACAAAGGTGAAGACATTCAACTTCT
TAAGAGTGCTTATGAAAATTTTAATCAGCACGAAGTTCTACTTGCACCATTATTAT
CAGCTGGTATTTTTGGTGCTGACCCTATACATTCTTTAAGAGTTTGTGTAGATAC
TGTTCGCACAAATGTCTACTTAGCTGTCTTTGATAAAAATCTCTATGACAAACTTG
TTTCAAGCTTTTTGGAAATGAAGAGTGAAAAGCAAGTTGAACAAAAGATCGCTGA
GATTCCTAAAGAGGAAGTTAAGCCATTTATAACTGAAAGTAAACCTTCAGTTGAA
CAGAGAAAACAAGATGATAAGAAAATCAAAGCTTGTGTTGAAGAAGTTACAACAA
CTCTGGAAGAAACTAAGTTCCTCACAGAAAACTTGTTACTTTATATTGACATTAAT
GGCAATCTTCATCCAGATTCTGCCACTCTTGTTAGTGACATTGACATCACTTTCT
TAAAGAAAGATGCTCCATATATAGTGGGTGATGTTGTTCAAGAGGGTGTTTTAAC
TGCTGTGGTTATACCTACTAAAAAGGCTGGTGGCACTACTGAAATGCTAGCGAA
AGCTTTGAGAAAAGTGCCAACAGACAATTATATAACCACTTACCCGGGTCAGGG
TTTAAATGGTTACACTGTAGAGGAGGCAAAGACAGTGCTTAAAAAGTGTAAAAGT
GCCTTTTACATTCTACCATCTATTATCTCTAATGAGAAGCAAGAAATTCTTGGAAC
TGTTTCTTGGAATTTGCGAGAAATGCTTGCACATGCAGAAGAAACACGCAAATTA
ATGCCTGTCTGTGTGGAAACTAAAGCCATAGTTTCAACTATACAGCGTAAATATA
AGGGTATTAAAATACAAGAGGGTGTGGTTGATTATGGTGCTAGATTTTACTTTTA
CACCAGTAAAACAACTGTAGCGTCACTTATCAACACACTTAACGATCTAAATGAA
ACTCTTGTTACAATGCCACTTGGCTATGTAACACATGGCTTAAATTTGGAAGAAG
CTGCTCGGTATATGAGATCTCTCAAAGTGCCAGCTACAGTTTCTGTTTCTTCACC
TGATGCTGTTACAGCGTATAATGGTTATCTTACTTCTTCTTCTAAAACACCTGAAG
AACATTTTATTGAAACCATCTCACTTGCTGGTTCCTATAAAGATTGGTCCTATTCT
GGACAATCTACACAACTAGGTATAGAATTTCTTAAGAGAGGTGATAAAAGTGTAT
ATTACACTAGTAATCCTACCACATTCCACCTAGATGGTGAAGTTATCACCTTTGA
CAATCTTAAGACACTTCTTTCTTTGAGAGAAGTGAGGACTATTAAGGTGTTTACA
ACAGTAGACAACATTAACCTCCACACGCAAGTTGTGGACATGTCAATGACATAT
GGACAACAGTTTGGTCCAACTTATTTGGATGGAGCTGATGTTACTAAAATAAAAC
CTCATAATTCACATGAAGGTAAAACATTTTATGTTTTACCTAATGATGACACTCTA
CGTGTTGAGGCTTTTGAGTACTACCACACAACTGATCCTAGTTTTCTGGGTAGGT
ACATGTCAGCATTAAATCACACTAAAAAGTGGAAATACCCACAAGTTAATGGTTT
AACTTCTATTAAATGGGCAGATAACAACTGTTATCTTGCCACTGCATTGTTAACA
CTCCAACTAATAGAGTTGTGTTTAATCCACCTGCTCTACAAGATGCTTATTACA
GAGCAAGGGCTGGTGAAGCTGCTAACTTTTGTGCACTTATCTTAGCCTACTGTA
ATAAGACAGTAGGTGAGTTAGGTGATGTTAGAGAAACAATGAGTTACTTGTTTCA
ACATGCCAATTTAGATTCTTGCAAAAGAGTCTTGAACGTGGTGTGTAAAACTTGT
GGACAACAGCAGACAACCCTTAAGGGTGTAGAAGCTGTTATGTACATGGGCACA
CTTTCTTATGAACAATTTAAGAAAGGTGTTCAGATACCTTGTACGTGTGGTAAAC
AAGCTACAAAATATCTAGTACAACAGGAGTCACCTTTTGTTATGATGTCAGCACC
ACCTGCTCAGTATGAACTTAAGCATGGTACATTTACTTGTGCTAGTGAGTACACT
GGTAATTACCAGTGTGGTCACTATAAACATATAACTTCTAAAGAAACTTTGTATTG
CATAGACGGTGCTTTACTTACAAAGTCCTCAGAATACAAAGGTCCTATTACGGAT
GTTTTCTACAAAGAAAACAGTTACACAACAACCATAAAACCAGTTACTTATAAATT
GGATGGTGTTGTTTGTACAGAAATTGACCCTAAGTTGGACAATTATTATAAGAAA
GACAATTCTTATTTCACAGAGCAACCAATTGATCTTGTACCAAACCAACCATATC
CAAACGCAAGCTTCGATAATTTTAAGTTTGTATGTGATAATATCAAATTTGCTGAT
GATTTAAACCAGTTAACTGGTTATAAGAAACCTGCTTCAAGAGAGCTTAAAGTTA
CATTTTTCCCTGACTTAAATGGTGATGTGGTGGCTATTGATTATAAACACTACAC
ACCCTCTTTTAAGAAAGGAGCTAAATTGTTACATAAACCTATTGTTTGGCATGTTA
ACAATGCAACTAATAAAGCCACGTATAAACCAAATACCTGGTGTATACGTTGTCT
TTGGAGCACAACCAGTTGAAACATCAAATTCGTTTGATGTACTGAAGTCAGAG
GACGCGCAGGGAATGGATAATCTTGCCTGCGAAGATCTAAAACCAGTCTCTGAA
GAAGTAGTGGAAAATCCTACCATACAGAAAGACGTTCTTGAGTGTAATGTGAAAA
CTACCGAAGTTGTAGGAGACATTATACTTAAACCAGCAAATAATAGTTTAAAAATT
ACAGAAGAGGTTGGCCACACAGATCTAATGGCTGCTTATGTAGACAATTCTAGT
CTTACTATTAAGAAACCTAATGAATTATCTAGAGTATTAGGTTTGAAAACCCTTGC
TACTCATGGTTTAGCTGCTGTTAATAGTGTCCCTTGGGATACTATAGCTAATTAT
GCTAAGCCTTTTCTTAACAAAGTTGTTAGTACAACTACTAACATAGTTACACGGT
GTTTAAACCGTGTTTGTACTAATTATATGCCTTATTTCTTTACTTTATTGCTACAAT
TGTGTACTTTTACTAGAAGTACAAATTCTAGAATTAAAGCATCTATGCCGACTACT
ATAGCAAAGAATACTGTTAAGAGTGTCGGTAAATTTTGTCTAGAGGCTTCATTTA
ATTATTTGAAGTCACCTAATTTTTCTAAACTGATAAATATTATAATTTGGTTTTTAC
TATTAAGTGTTTGCCTAGGTTCTTTAATCTACTCAACCGCTGCTTTAGGTGTTTTA
ATGTCTAATTTAGGCATGCCTTCTTACTGTACTGGTTACAGAGAAGGCTATTTGA
ACTCTACTAATGTCACTATTGCAACCTACTGTACTGGTTCTATACCTTGTAGTGTT
TGTCTTAGTGGTTTAGATTCTTTAGACACCTATCCTTCTTTAGAAACTATACAAAT
TACCATTTCATCTTTTAAATGGGATTTAACTGCTTTTGGCTTAGTTGCAGAGTGGT
TTTTGGCATATATTCTTTTCACTAGGTTTTTCTATGTACTTGGATTGGCTGCAATC
ATGCAATTGTTTTTCAGCTATTTTGCAGTACATTTTATTAGTAATTCTTGGCTTAT
GTGGTTAATAATTAATCTTGTACAAATGGCCCCGATTTCAGCTATGGTTAGAATG
TACATCTTCTTTGCATCATTTTATTATGTATGGAAAAGTTATGTGCATGTTGTAGA
CGGTTGTAATTCATCAACTTGTATGATGTGTTACAAACGTAATAGAGCAACAAGA
GTCGAATGTACAACTATTGTTAATGGTGTTAGAAGGTCCTTTTATGTCTATGCTA
ATGGAGGTAAAGGCTTTTGCAAACTACACAATTGGAATTGTGTTAATTGTGATAC
ATTCTGTGCTGGTAGTACATTTATTAGTGATGAAGTTGCGAGAGACTTGTCACTA
CAGTTTAAAAGACCAATAAATCCTACTGACCAGTCTTCTTACATCGTTGATAGTG
TTACAGTGAAGAATGGTTCCATCCATCTTTACTTTGATAAAGCTGGTCAAAAGAC
TTATGAAAGACATTCTCTCTCTCATTTTGTTAACTTAGACAACCTGAGAGCTAATA
ACACTAAAGGTTCATTGCCTATTAATGTTATAGTTTTTGATGGTAAATCAAAATGT
GAAGAATCATCTGCAAAATCAGCGTCTGTTTACTACAGTCAGCTTATGTGTCAAC
CTATACTGTTACTAGATCAGGCATTAGTGTCTGATGTTGGTGATAGTGCGGAAGT
TGCAGTTAAAATGTTTGATGCTTACGTTAATACGTTTTCATCAACTTTTAACGTAC
CAATGGAAAAACTCAAAACACTAGTTGCAACTGCAGAAGCTGAACTTGCAAAGA
ATGTGTCCTTAGACAATGTCTTATCTACTTTTATTTCAGCAGCTCGGCAAGGGTT
TGTTGATTCAGATGTAGAAACTAAAGATGTTGTTGAATGTCTTAAATTGTCACATC
AATCTGACATAGAAGTTACTGGCGATAGTTGTAATAACTATATGCTCACCTATAA
CAAAGTTGAAAACATGACACCCCGTGACCTTGGTGCTTGTATTGACTGTAGTGC
GCGTCATATTAATGCGCAGGTAGCAAAAAGTCACAACATTGCTTTGATATGGAAC
GTTAAAGATTTCATGTCATTGTCTGAACAACTACGAAAACAAATACGTAGTGCTG
CTAAAAAGAATAACTTACCTTTTAAGTTGACATGTGCAACTACTAGACAAGTTGTT
AATGTTGTAACAACAAAGATAGCACTTAAGGGTGGTAAAATTGTTAATAATTGGT
TGAAGCAGTTAATTAAAGTTACACTTGTGTTCCTTTTTGTTGCTGCTATTTTCTAT
TTAATAACACCTGTTCATGTCATGTCTAAACATACTGACTTTTCAAGTGAAATCAT
AGGATACAAGGCTATTGATGGTGGTGTCACTCGTGACATAGCATCTACAGATAC
TTGTTTTGCTAACAAACATGCTGATTTTGACACATGGTTTAGCCAGCGTGGTGGT
AGTTATACTAATGACAAAGCTTGCCCATTGATTGCTGCAGTCATAACAAGAGAAG
TGGGTTTTGTCGTGCCTGGTTTGCCTGGCACGATATTACGCACAACTAATGGTG
ACTTTTTGCATTTCTTACCTAGAGTTTTTAGTGCAGTTGGTAACATCTGTTACACA
CCATCAAAACTTATAGAGTACACTGACTTTGCAACATCAGCTTGTGTTTTGGCTG
CTGAATGTACAATTTTTAAAGATGCTTCTGGTAAGCCAGTACCATATTGTTATGAT
ACCAATGTACTAGAAGGTTCTGTTGCTTATGAAAGTTTACGCCCTGACACACGTT
ATGTGCTCATGGATGGCTCTATTATTCAATTTCCTAACACCTACCTTGAAGGTTC
TGTTAGAGTGGTAACAACTTTTGATTCTGAGTACTGTAGGCACGGCACTTGTGAA
AGATCAGAAGCTGGTGTTTGTGTATCTACTAGTGGTAGATGGGTACTTAACAATG
ATTATTACAGATCTTTACCAGGAGTTTTCTGTGGTGTAGATGCTGTAAATTTACTT
ACTAATATGTTTACACCACTAATTCAACCTATTGGTGCTTTGGACATATCAGCATC
TATAGTAGCTGGTGGTATTGTAGCTATCGTAGTAACATGCCTTGCCTACTATTTT
ATGAGGTTTAGAAGAGCTTTTGGTGAATACAGTCATGTAGTTGCCTTTAATACTT
TACTATTCCTTATGTCATTCACTGTACTCTGTTTAACACCAGTTTACTCATTCTTA
CCTGGTGTTTATTCTGTTATTTACTTGTACTTGACATTTTATCTTACTAATGATGTT
TCTTTTTTAGCACATATTCAGTGGATGGTTATGTTCACACCTTTAGTACCTTTCTG
GATAACAATTGCTTATATCATTTGTATTTCCACAAAGCATTTCTATTGGTTCTTTA
GTAATTACCTAAAGAGACGTGTAGTCTTTAATGGTGTTTCCTTTAGTACTTTTGAA
GAAGCTGCGCTGTGCACCTTTTTGTTAAATAAAGAAATGTATCTAAAGTTGCGTA
GTGATGTGCTATTACCTCTTACGCAATATAATAGATACTTAGCTCTTTATAATAAG
TACAAGTATTTTAGTGGAGCAATGGATACAACTAGCTACAGAGAAGCTGCTTGTT
GTCATCTCGCAAAGGCTCTCAATGACTTCAGTAACTCAGGTTCTGATGTTCTTTA
CCAACCACCACAAACCTCTATCACCTCAGCTGTTTTGCAGAGTGGTTTTAGAAAA
ATGGCATTCCCATCTGGTAAAGTTGAGGGTTGTATGGTACAAGTAACTTGTGGT
ACAACTACACTTAACGGTCTTTGGCTTGATGACGTAGTTTACTGTCCAAGACATG
TGATCTGCACCTCTGAAGACATGCTTAACCCTAATTATGAAGATTTACTCATTCG
TAAGTCTAATCATAATTTCTTGGTACAGGCTGGTAATGTTCAACTCAGGGTTATT
GGACATTCTATGCAAAATTGTGTACTTAAGCTTAAGGTTGATACAGCCAATCCTA
AGACACCTAAGTATAAGTTTGTTCGCATTCAACCAGGACAGACTTTTTCAGTGTT
AGCTTGTTACAATGGTTCACCATCTGGTGTTTACCAATGTGCTATGAGGCCCAAT
TTCACTATTAAGGGTTCATTCCTTAATGGTTCATGTGGTAGTGTTGGTTTTAACAT
AGATTATGACTGTGTCTCTTTTTGTTACATGCACCATATGGAATTACCAACTGGA
GTTCATGCTGGCACAGACTTAGAAGGTAACTTTTATGGACCTTTTGTTGACAGGC
AAACAGCACAAGCAGCTGGTACGGACACAACTATTACAGTTAATGTTTTAGCTTG
GTTGTACGCTGCTGTTATAAATGGAGACAGGTGGTTTCTCAATCGATTTACCACA
ACTCTTAATGACTTTAACCTTGTGGCTATGAAGTACAATTATGAACCTCTAACACA
AGACCATGTTGACATACTAGGACCTCTTTCTGCTCAAACTGGAATTGCCGTTTTA
GATATGTGTGCTTCATTAAAAGAATTACTGCAAAATGGTATGAATGGACGTACCA
TATTGGGTAGTGCTTTATTAGAAGATGAATTTACACCTTTTGATGTTGTTAGACAA
TGCTCAGGTGTTACTTTCCAAAGTGCAGTGAAAAGAACAATCAAGGGTACACAC
CACTGGTTGTTACTCACAATTTTGACTTCACTTTTAGTTTTAGTCCAGAGTACTCA
ATGGTCTTTGTTCTTTTTTTTGTATGAAAATGCCTTTTTACCTTTTGCTATGGGTAT
TATTGCTATGTCTGCTTTTGCAATGATGTTTGTCAAACATAAGCATGCATTTCTCT
GTTTGTTTTTGTTACCTTCTCTTGCCACTGTAGCTTATTTTAATATGGTCTATATG
CCTGCTAGTTGGGTGATGCGTATTATGACATGGTTGGATATGGTTGATACTAGTT
TGTCTGGTTTTAAGCTAAAAGACTGTGTTATGTATGCATCAGCTGTAGTGTTACT
AATCCTTATGACAGCAAGAACTGTGTATGATGATGGTGCTAGGAGAGTGTGGAC
ACTTATGAATGTCTTGACACTCGTTTATAAAGTTTATTATGGTAATGCTTTAGATC
AAGCCATTTCCATGTGGGCTCTTATAATCTCTGTTACTTCTAACTACTCAGGTGT
AGTTACAACTGTCATGTTTTTGGCCAGAGGTATTGTTTTTATGTGTGTTGAGTATT
GCCCTATTTTCTTCATAACTGGTAATACACTTCAGTGTATAATGCTAGTTTATTGT
TTCTTAGGCTATTTTTGTACTTGTTACTTTGGCCTCTTTTGTTTACTCAACCGCTA
CTTTAGACTGACTCTTGGTGTTTATGATTACTTAGTTTCTACACAGGAGTTTAGAT
ATATGAATTCACAGGGACTACTCCCACCCAAGAATAGCATAGATGCCTTCAAACT
CAACATTAAATTGTTGGGTGTTGGTGGCAAACCTTGTATCAAAGTAGCCACTGTA
CAGTCTAAAATGTCAGATGTAAAGTGCACATCAGTAGTCTTACTCTCAGTTTTGC
AACAACTCAGAGTAGAATCATCATCTAAATTGTGGGCTCAATGTGTCCAGTTACA
CAATGACATTCTCTTAGCTAAAGATACTACTGAAGCCTTTGAAAAAATGGTTTCA
CTACTTTCTGTTTTGCTTTCCATGCAGGGTGCTGTAGACATAAACAAGCTTTGTG
AAGAAATGCTGGACAACAGGGCAACCTTACAAGCTATAGCCTCAGAGTTTAGTT
CCCTTCCATCATATGCAGCTTTTGCTACTGCTCAAGAAGCTTATGAGCAGGCTGT
TGCTAATGGTGATTCTGAAGTTGTTCTTAAAAAGTTGAAGAAGTCTTTGAATGTG
GCTAAATCTGAATTTGACCGTGATGCAGCCATGCAACGTAAGTTGGAAAAGATG
GCTGATCAAGCTATGACCCAAATGTATAAACAGGCTAGATCTGAGGACAAGAGG
GCAAAAGTTACTAGTGCTATGCAGACAATGCTTTTCACTATGCTTAGAAAGTTGG
ATAATGATGCACTCAACAACATTATCAACAATGCAAGAGATGGTTGTGTTCCCTT
GAACATAATACCTCTTACAACAGCAGCCAAACTAATGGTTGTCATACCAGACTAT
AACACATATAAAAATACGTGTGATGGTACAACATTTACTTATGCATCAGCATTGT
GGGAAATCCAACAGGTTGTAGATGCAGATAGTAAAATTGTTCAACTTAGTGAAAT
TAGTATGGACAATTCACCTAATTTAGCATGGCCTCTTATTGTAACAGCTTTAAGG
GCCAATTCTGCTGTCAAATTACAGAATAATGAGCTTAGTCCTGTTGCACTACGAC
AGATGTCTTGTGCTGCCGGTACTACACAAACTGCTTGCACTGATGACAATGCGT
TAGCTTACTACAACACAACAAAGGGAGGTAGGTTTGTACTTGCACTGTTATCCGA
TTTACAGGATTTGAAATGGGCTAGATTCCCTAAGAGTGATGGAACTGGTACTATC
TATACAGAACTGGAACCACCTTGTAGGTTTGTTACAGACACACCTAAAGGTCCTA
AAGTGAAGTATTTATACTTTATTAAAGGATTAAACAACCTAAATAGAGGTATGGTA
CTTGGTAGTTTAGCTGCCACAGTACGTCTACAAGCTGGTAATGCAACAGAAGTG
CCTGCCAATTCAACTGTATTATCTTTCTGTGCTTTTGCTGTAGATGCTGCTAAAG
CTTACAAAGATTATCTAGCTAGTGGGGGACAACCAATCACTAATTGTGTTAAGAT
GTTGTGTACACACACTGGTACTGGTCAGGCAATAACAGTTACACCGGAAGCCAA
TATGGATCAAGAATCCTTTGGTGGTGCATCGTGTTGTCTGTACTGCCGTTGCCA
CATAGATCATCCAAATCCTAAAGGATTTTGTGACTTAAAAGGTAAGTATGTACAA
ATACCTACAACTTGTGCTAATGACCCTGTGGGTTTTACACTTAAAAACACAGTCT
GTACCGTCTGCGGTATGTGGAAAGGTTATGGCTGTAGTTGTGATCAACTCCGCG
AACCCATGCTTCAGTCAGCTGATGCACAATCGTTTTTAAACGGGTTTGCGGTGT
AAGTGCAGCCCGTCTTACACCGTGCGGCACAGGCACTAGTACTGATGTCGTATA
CAGGGCTTTTGACATCTACAATGATAAAGTAGCTGGTTTTGCTAAATTCCTAAAA
ACTAATTGTTGTCGCTTCCAAGAAAAGGACGAAGATGACAATTTAATTGATTCTT
ACTTTGTAGTTAAGAGACACACTTTCTCTAACTACCAACATGAAGAAACAATTTAT
AATTTACTTAAGGATTGTCCAGCTGTTGCTAAACATGACTTCTTTAAGTTTAGAAT
AGACGGTGACATGGTACCACATATATCACGTCAACGTCTTACTAAATACACAATG
GCAGACCTCGTCTATGCTTTAAGGCATTTTGATGAAGGTAATTGTGACACATTAA
AAGAAATACTTGTCACATACAATTGTTGTGATGATGATTATTTCAATAAAAAGGAC
TGGTATGATTTTGTAGAAAACCCAGATATATTACGCGTATACGCCAACTTAGGTG
AACGTGTACGCCAAGCTTTGTTAAAAACAGTACAATTCTGTGATGCCATGCGAAA
TGCTGGTATTGTTGGTGTACTGACATTAGATAATCAAGATCTCAATGGTAACTGG
TATGATTTCGGTGATTTCATACAAACCACGCCAGGTAGTGGAGTTCCTGTTGTAG
ATTCTTATTATTCATTGTTAATGCCTATATTAACCTTGACCAGGGCTTTAACTGCA
GAGTCACATGTTGACACTGACTTAACAAAGCCTTACATTAAGTGGGATTTGTTAA
AATATGACTTCACGGAAGAGAGGTTAAAACTCTTTGACCGTTATTTTAAATATTG
GGATCAGACATACCACCCAAATTGTGTTAACTGTTTGGATGACAGATGCATTCTG
CATTGTGCAAACTTTAATGTTTTATTCTCTACAGTGTTCCCACCTACAAGTTTTGG
ACCACTAGTGAGAAAAATATTTGTTGATGGTGTTCCATTTGTAGTTTCAACTGGA
TACCACTTCAGAGAGCTAGGTGTTGTACATAATCAGGATGTAAACTTACATAGCT
CTAGACTTAGTTTTAAGGAATTACTTGTGTATGCTGCTGACCCTGCTATGCACGC
TGCTTCTGGTAATCTATTACTAGATAAACGCACTACGTGCTTTTCAGTAGCTGCA
CTTACTAACAATGTTGCTTTTCAAACTGTCAAACCCGGTAATTTTAACAAAGACTT
CTATGACTTTGCTGTGTCTAAGGGTTTCTTTAAGGAAGGAAGTTCTGTTGAATTA
AAACACTTCTTCTTTGCTCAGGATGGTAATGCTGCTATCAGCGATTATGACTACT
ATCGTTATAATCTACCAACAATGTGTGATATCAGACAACTACTATTTGTAGTTGAA
GTTGTTGATAAGTACTTTGATTGTTACGATGGTGGCTGTATTAATGCTAACCAAG
TCATCGTCAACAACCTAGACAAATCAGCTGGTTTTCCATTTAATAAATGGGGTAA
GGCTAGACTTTATTATGATTCAATGAGTTATGAGGATCAAGATGCACTTTTCGCA
TATACAAAACGTAATGTCATCCCTACTATAACTCAAATGAATCTTAAGTATGCCAT
TAGTGCAAAGAATAGAGCTCGCACCGTAGCTGGTGTCTCTATCTGTAGTACTAT
GACCAATAGACAGTTTCATCAAAAATTATTGAAATCAATAGCCGCCACTAGAGGA
GCTACTGTAGTAATTGGAACAAGCAAATTCTATGGTGGTTGGCACAACATGTTAA
AAACTGTTTATAGTGATGTAGAAAACCCTCACCTTATGGGTTGGGATTATCCTAA
ATGTGATAGAGCCATGCCTAACATGCTTAGAATTATGGCCTCACTTGTTCTTGCT
CGCAAACATACAACGTGTTGTAGCTTGTCACACCGTTTCTATAGATTAGCTAATG
AGTGTGCTCAAGTATTGAGTGAAATGGTCATGTGTGGCGGTTCACTATATGTTAA
ACCAGGTGGAACCTCATCAGGAGATGCCACAACTGCTTATGCTAATAGTGTTTTT
AACATTTGTCAAGCTGTCACGGCCAATGTTAATGCACTTTTATCTACTGATGGTA
ACAAAATTGCCGATAAGTATGTCCGCAATTTACAACACAGACTTTATGAGTGTCT
CTATAGAAATAGAGATGTTGACACAGACTTTGTGAATGAGTTTTACGCATATTTG
CGTAAACATTTCTCAATGATGATACTCTCTGACGATGCTGTTGTGTGTTTCAATA
GCACTTATGCATCTCAAGGTCTAGTGGCTAGCATAAAGAACTTTAAGTCAGTTCT
TTATTATCAAAACAATGTTTTTATGTCTGAAGCAAAATGTTGGACTGAGACTGAC
CTTACTAAAGGACCTCATGAATTTTGCTCTCAACATACAATGCTAGTTAAACAGG
GTGATGATTATGTGTACCTTCCTTACCCAGATCCATCAAGAATCCTAGGGGCCG
GCTGTTTTGTAGATGATATCGTAAAAACAGATGGTACACTTATGATTGAACGGTT
CGTGTCTTTAGCTATAGATGCTTACCCACTTACTAAACATCCTAATCAGGAGTAT
GCTGATGTCTTTCATTTGTACTTACAATACATAAGAAAGCTACATGATGAGTTAAC
AGGACACATGTTAGACATGTATTCTGTTATGCTTACTAATGATAACACTTCAAGG
TATTGGGAACCTGAGTTTTATGAGGCTATGTACACACCGCATACAGTCTTACAG
GCTGTTGGGGCTTGTGTTCTTTGCAATTCACAGACTTCATTAAGATGTGGTGCTT
GCATACGTAGACCATTCTTATGTTGTAAATGCTGTTACGACCATGTCATATCAAC
ATCACATAAATTAGTCTTGTCTGTTAATCCGTATGTTTGCAATGCTCCAGGTTGT
GATGTCACAGATGTGACTCAACTTTACTTAGGAGGTATGAGCTATTATTGTAAAT
CACATAAACCACCCATTAGTTTTCCATTGTGTGCTAATGGACAAGTTTTTGGTTTA
TATAAAAATACATGTGTTGGTAGCGATAATGTTACTGACTTTAATGCAATTGCAAC
ATGTGACTGGACAAATGCTGGTGATTACATTTTAGCTAACACCTGTACTGAAAGA
CTCAAGCTTTTTGCAGCAGAAACGCTCAAAGCTACTGAGGAGACATTTAAACTGT
CTTATGGTATTGCTACTGTACGTGAAGTGCTGTCTGACAGAGAATTACATCTTTC
ATGGGAAGTTGGTAAACCTAGACCACCACTTAACCGAAATTATGTCTTTACTGGT
TATCGTGTAACTAAAAACAGTAAAGTACAAATAGGAGAGTACACCTTTGAAAAAG
GTGACTATGGTGATGCTGTTGTTTACCGAGGTACAACAACTTACAAATTAAATGT
TGGTGATTATTTTGTGCTGACATCACATACAGTAATGCCATTAAGTGCACCTACA
CTAGTGCCACAAGAGCACTATGTTAGAATTACTGGCTTATACCCAACACTCAATA
TCTCAGATGAGTTTTCTAGCAATGTTGCAAATTATCAAAAGGTTGGTATGCAAAA
GTATTCTACACTCCAGGGACCACCTGGTACTGGTAAGAGTCATTTTGCTATTGG
CCTAGCTCTCTACTACCCTTCTGCTCGCATAGTGTATACAGCTTGCTCTCATGCC
GCTGTTGATGCACTATGTGAGAAGGCATTAAAATATTTGCCTATAGATAAATGTA
GTAGAATTATACCTGCACGTGCTCGTGTAGAGTGTTTTGATAAATTCAAAGTGAA
TTCAACATTAGAACAGTATGTCTTTTGTACTGTAAATGCATTGCCTGAGACGACA
GCAGATATAGTTGTCTTTGATGAAATTTCAATGGCCACAAATTATGATTTGAGTG
TTGTCAATGCCAGATTACGTGCTAAGCACTATGTGTACATTGGCGACCCTGCTC
AATTACCTGCACCACGCACATTGCTAACTAAGGGCACACTAGAACCAGAATATTT
CAATTCAGTGTGTAGACTTATGAAAACTATAGGTCCAGACATGTTCCTCGGAACT
TGTCGGCGTTGTCCTGCTGAAATTGTTGACACTGTGAGTGCTTTGGTTTATGATA
ATAAGCTTAAAGCACATAAAGACAAATCAGCTCAATGCTTTAAAATGTTTTATAAG
GGTGTTATCACGCATGATGTTTCATCTGCAATTAACAGGCCACAAATAGGCGTG
GTAAGAGAATTCCTTACACGTAACCCTGCTTGGAGAAAAGCTGTCTTTATTTCAC
CTTATAATTCACAGAATGCTGTAGCCTCAAAGATTTTGGGACTACCAACTCAAAC
TGTTGATTCATCACAGGGCTCAGAATATGACTATGTCATATTCACTCAAACCACT
GAAACAGCTCACTCTTGTAATGTAAACAGATTTAATGTTGCTATTACCAGAGCAA
AAGTAGGCATACTTTGCATAATGTCTGATAGAGACCTTTATGACAAGTTGCAATT
TACAAGTCTTGAAATTCCACGTAGGAATGTGGCAACTTTACAAGCTGAAAATGTA
ACAGGACTCTTTAAAGATTGTAGTAAGGTAATCACTGGGTTACATCCTACACAGG
CACCTACACACCTCAGTGTTGACACTAAATTCAAAACTGAAGGTTTATGTGTTGA
CATACCTGGCATACCTAAGGACATGACCTATAGAAGACTCATCTCTATGATGGG
TTTTAAAATGAATTATCAAGTTAATGGTTACCCTAACATGTTTATCACCCGCGAAG
AAGCTATAAGACATGTACGTGCATGGATTGGCTTCGATGTCGAGGGGTGTCATG
CTACTAGAGAAGCTGTTGGTACCAATTTACCTTTACAGCTAGGTTTTTCTACAGG
TGTTAACCTAGTTGCTGTACCTACAGGTTATGTTGATACACCTAATAATACAGATT
TTTCCAGAGTTAGTGCTAAACCACCGCCTGGAGATCAATTTAAACACCTCATACC
ACTTATGTACAAAGGACTTCCTTGGAATGTAGTGCGTATAAAGATTGTACAAATG
TTAAGTGACACACTTAAAAATCTCTCTGACAGAGTCGTATTTGTCTTATGGGCAC
ATGGCTTTGAGTTGACATCTATGAAGTATTTTGTGAAAATAGGACCTGAGCGCAC
CTGTTGTCTATGTGATAGACGTGCCACATGCTTTTCCACTGCTTCAGACACTTAT
GCCTGTTGGCATCATTCTATTGGATTTGATTACGTCTATAATCCGTTTATGATTGA
TGTTCAACAATGGGGTTTTACAGGTAACCTACAAAGCAACCATGATCTGTATTGT
CAAGTCCATGGTAATGCACATGTAGCTAGTTGTGATGCAATCATGACTAGGTGT
CTAGCTGTCCACGAGTGCTTTGTTAAGCGTGTTGACTGGACTATTGAATATCCTA
TAATTGGTGATGAACTGAAGATTAATGCGGCTTGTAGAAAGGTTCAACACATGGT
TGTTAAAGCTGCATTATTAGCAGACAAATTCCCAGTTCTTCACGACATTGGTAAC
CCTAAAGCTATTAAGTGTGTACCTCAAGCTGATGTAGAATGGAAGTTCTATGATG
CACAGCCTTGTAGTGACAAAGCTTATAAAATAGAAGAATTATTCTATTCTTATGCC
ACACATTCTGACAAATTCACAGATGGTGTATGCCTATTTTGGAATTGCAATGTCG
ATAGATATCCTGCTAATTCCATTGTTTGTAGATTTGACACTAGAGTGCTATCTAAC
CTTAACTTGCCTGGTTGTGATGGTGGCAGTTTGTATGTAAATAAACATGCATTCC
ACACACCAGCTTTTGATAAAAGTGCTTTTGTTAATTTAAAACAATTACCATTTTTC
TATTACTCTGACAGTCCATGTGAGTCTCATGGAAAACAAGTAGTGTCAGATATAG
ATTATGTACCACTAAAGTCTGCTACGTGTATAACACGTTGCAATTTAGGTGGTGC
TGTCTGTAGACATCATGCTAATGAGTACAGATTGTATCTCGATGCTTATAACATG
ATGATCTCAGCTGGCTTTAGCTTGTGGGTTTACAAACAATTTGATACTTATAACC
TCTGGAACACTTTTACAAGACTTCAGAGTTTAGAAAATGTGGCTTTTAATGTTGTA
AATAAGGGACACTTTGATGGACAACAGGGTGAAGTACCAGTTTCTATCATTAATA
ACACTGTTTACACAAAAGTTGATGGTGTTGATGTAGAATTGTTTGAAAATAAAAC
AACATTACCTGTTAATGTAGCATTTGAGCTTTGGGCTAAGCGCAACATTAAACCA
GTACCAGAGGTGAAAATACTCAATAATTTGGGTGTGGACATTGCTGCTAATACTG
TGATCTGGGACTACAAAAGAGATGCTCCAGCACATATATCTACTATTGGTGTTTG
TTCTATGACTGACATAGCCAAGAAACCAACTGAAACGATTTGTGCACCACTCACT
GTCTTTTTTGATGGTAGAGTTGATGGTCAAGTAGACTTATTTAGAAATGCCCGTA
ATGGTGTTCTTATTACAGAAGGTAGTGTTAAAGGTTTACAACCATCTGTAGGTCC
CAAACAAGCTAGTCTTAATGGAGTCACATTAATTGGAGAAGCCGTAAAAACACA
GTTCAATTATTATAAGAAAGTTGATGGTGTTGTCCAACAATTACCTGAAACTTACT
TTACTCAGAGTAGAAATTTACAAGAATTTAAACCCAGGAGTCAAATGGAAATTGA
TTTCTTAGAATTAGCTATGGATGAATTCATTGAACGGTATAAATTAGAAGGCTAT
GCCTTCGAACATATCGTTTATGGAGATTTTAGTCATAGTCAGTTAGGTGGTTTAC
ATCTACTGATTGGACTAGCTAAACGTTTTAAGGAATCACCTTTTGAATTAGAAGA
TTTTATTCCTATGGACAGTACAGTTAAAAACTATTTCATAACAGATGCGCAAACA
GGTTCATCTAAGTGTGTGTGTTCTGTTATTGATTTATTACTTGATGATTTTGTTGA
AATAATAAAATCCCAAGATTTATCTGTAGTTTCTAAGGTTGTCAAAGTGACTATTG
ACTATACAGAAATTTCATTTATGCTTTGGTGTAAAGATGGCCATGTAGAAACATTT
TACCCAAAATTACAATCTAGTCAAGCGTGGCAACCGGGTGTTGCTATGCCTAAT
CTTTACAAAATGCAAAGAATGCTATTAGAAAAGTGTGACCTTCAAAATTATGGTG
ATAGTGCAACATTACCTAAAGGCATAATGATGAATGTCGCAAAATATACTCAACT
GTGTCAATATTTAAACACATTAACATTAGCTGTACCCTATAATATGAGAGTTATAC
ATTTTGGTGCTGGTTCTGATAAAGGAGTTGCACCAGGTACAGCTGTTTTAAGACA
GTGGTTGCCTACGGGTACGCTGCTTGTCGATTCAGATCTTAATGACTTTGTCTCT
GATGCAGATTCAACTTTGATTGGTGATTGTGCAACTGTACATACAGCTAATAAAT
GGGATCTCATTATTAGTGATATGTACGACCCTAAGACTAAAAATGTTACAAAAGA
AAATGACTCTAAAGAGGGTTTTTTCACTTACATTTGTGGGTTTATACAACAAAAG
CTAGCTCTTGGAGGTTCCGTGGCTATAAAGATAACAGAACATTCTTGGAATGCT
GATCTTTATAAGCTCATGGGACACTTCGCATGGTGGACAGCCTTTGTTACTAATG
TGAATGCGTCATCATCTGAAGCATTTTTAATTGGATGTAATTATCTTGGCAAACC
ACGCGAACAAATAGATGGTTATGTCATGCATGCAAATTACATATTTTGGAGGAAT
ACAAATCCAATTCAGTTGTCTTCCTATTCTTTATTTGACATGAGTAAATTTCCCCT
TAAATTAAGGGGTACTGCTGTTATGTCTTTAAAAGAAGGTCAAATCAATGATATG
ATTTTATCTCTTCTTAGTAAAGGTAGACTTATAATTAGAGAAAACAACAGAGTTGT
TATTTCTAGTGATGTTCTTGTTAACAACTAAACGAACAATGTTTGTTTTTCTTGTTT
TATTGCCACTAGTCTCTAGTCAGTGTGTTAATCTTACAACCAGAACTCAATTACC
CCCTGCATACACTAATTCTTTCACACGTGGTGTTTATTACCCTGACAAAGTTTTC
AGATCCTCAGTTTTACATTCAACTCAGGACTTGTTCTTACCTTTCTTTTCCAATGT
TACTTGGTTCCATGCTATACATGTCTCTGGGACCAATGGTACTAAGAGGTTTGAT
AACCCTGTCCTACCATTTAATGATGGTGTTTATTTTGCTTCCACTGAGAAGTCTA
ACATAATAAGAGGCTGGATTTTTGGTACTACTTTAGATTCGAAGACCCAGTCCCT
ACTTATTGTTAATAACGCTACTAATGTTGTTATTAAAGTCTGTGAATTTCAATTTTG
TAATGATCCATTTTTGGGTGTTTATTACCACAAAAACAACAAAAGTTGGATGGAA
AGTGAGTTCAGAGTTTATTCTAGTGCGAATAATTGCACTTTTGAATATGTCTCTCA
GCCTTTTCTTATGGACCTTGAAGGAAAACAGGGTAATTTCAAAAATCTTAGGGAA
TTTGTGTTTAAGAATATTGATGGTTATTTTAAAATATATTCTAAGCACACGCCTAT
TAATTTAGTGCGTGATCTCCCTCAGGGTTTTTCGGCTTTAGAACCATTGGTAGAT
TTGCCAATAGGTATTAACATCACTAGGTTTCAAACTTTACTTGCTTTACATAGAAG
TTATTTGACTCCTGGTGATTCTTCTTCAGGTTGGACAGCTGGTGCTGCAGCTTAT
TATGTGGGTTATCTTCAACCTAGGACTTTTCTATTAAAATATAATGAAAATGGAAC
CATTACAGATGCTGTAGACTGTGCACTTGACCCTCTCTCAGAAACAAAGTGTAC
GTTGAAATCCTTCACTGTAGAAAAAGGAATCTATCAAACTTCTAACTTTAGAGTC
CAACCAACAGAATCTATTGTTAGATTTCCTAATATTACAAACTTGTGCCCTTTTGG
TGAAGTTTTTAACGCCACCAGATTTGCATCTGTTTATGCTTGGAACAGGAAGAGA
ATCAGCAACTGTGTTGCTGATTATTCTGTCCTATATAATTCCGCATCATTTTCCAC
TTTTAAGTGTTATGGAGTGTCTCCTACTAAATTAAATGATCTCTGCTTTACTAATG
TCTATGCAGATTCATTTGTAATTAGAGGTGATGAAGTCAGACAAATCGCTCCAGG
GCAAACTGGAAAGATTGCTGATTATAATTATAAATTACCAGATGATTTTACAGGC
TGCGTTATAGCTTGGAATTCTAACAATCTTGATTCTAAGGTTGGTGGTAATTATAA
TTACCTGTATAGATTGTTTAGGAAGTCTAATCTCAAACCTTTTGAGAGAGATATTT
CAACTGAAATCTATCAGGCCGGTAGCACACCTTGTAATGGTGTTGAAGGTTTTAA
TTGTTACTTTCCTTTACAATCATATGGTTTCCAACCCACTAATGGTGTTGGTTACC
AACCATACAGAGTAGTAGTACTTTCTTTTGAACTTCTACATGCACCAGCAACTGT
TTGTGGACCTAAAAAGTCTACTAATTTGGTTAAAAACAAATGTGTCAATTTCAACT
TCAATGGTTTAACAGGCACAGGTGTTCTTACTGAGTCTAACAAAAAGTTTCTGCC
TTTCCAACAATTTGGCAGAGACATTGCTGACACTACTGATGCTGTCCGTGATCCA
CAGACACTTGAGATTCTTGACATTACACCATGTTCTTTTGGTGGTGTCAGTGTTA
TAACACCAGGAACAAATACTTCTAACCAGGTTGCTGTTCTTTATCAGGATGTTAA
CTGCACAGAAGTCCCTGTTGCTATTCATGCAGATCAACTTACTCCTACTTGGCGT
GTTTATTCTACAGGTTCTAATGTTTTTCAAACACGTGCAGGCTGTTTAATAGGGG
CTGAACATGTCAACAACTCATATGAGTGTGACATACCCATTGGTGCAGGTATATG
CGCTAGTTATCAGACTCAGACTAATTCTCCTCGGCGGGCACGTAGTGTAGCTAG
TCAATCCATCATTGCCTACACTATGTCACTTGGTGCAGAAAATTCAGTTGCTTAC
TCTAATAACTCTATTGCCATACCCACAAATTTTACTATTAGTGTTACCACAGAAAT
TCTACCAGTGTCTATGACCAAGACATCAGTAGATTGTACAATGTACATTTGTGGT
GATTCAACTGAATGCAGCAATCTTTTGTTGCAATATGGCAGTTTTTGTACACAATT
AAACCGTGCTTTAACTGGAATAGCTGTTGAACAAGACAAAAACCACCCAAGAAGTT
TTTGCACAAGTCAAACAAATTTACAAAACACCACCAATTAAAGATTTTGGTGGTTT
TAATTTTTCACAAATATTACCAGATCCATCAAAACCAAGCAAGAGGTCATTTATTG
AAGATCTACTTTTCAACAAAGTGACACTTGCAGATGCTGGCTTCATCAAACAATA
TGGTGATTGCCTTGGTGATATTGCTGCTAGAGACCTCATTTGTGCACAAACGTTT
AACGGCCTTACTGTTTTGCCACCTTTGCTCACAGATGAAATGATTGCTCAATACA
CTTCTGCACTGTTAGCGGGTACAATCACTTCTGGTTGGACCTTTGGTGCAGGTG
CTGCATTACAAATACCATTTGCTATGCAAATGGCTTATAGGTTTAATGGTATTGG
AGTTACACAGAATGTTCTCTATGAGAACCAAAAATTGATTGCCAACCAATTTAATA
GTGCTATTGGCAAAATTCAAGACTCACTTTCTTCCACAGCAAGTGCACTTGGAAA
ACTTCAAGATGTGGTCAACCAAAATGCACAAGCTTTAAACACGCTTGTTAAACAA
CTTAGCTCCAATTTTGGTGCAATTTCAAGTGTTTTAAATGATATCCTTTCACGTCT
TGACAAAGTTGAGGCTGAAGTGCAAATTGATAGGTTGATCACAGGCAGACTTCA
AAGTTTGCAGACATATGTGACTCAACAATTAATTAGAGCTGCAGAAATCAGAGCT
TCTGCTAATCTTGCTGCTACTAAAATGTCAGAGTGTGTACTTGGACAATCAAAAA
GAGTTGATTTTTGTGGAAAGGGCTATCATCTTATGTCCTTCCCTCAGTCAGCACC
TCATGGTGTAGTCTTCTTGCATGTGACTTATGTCCCTGCACAAGAAAAGAACTTC
ACAACTGCTCCTGCCATTTGTCATGATGGAAAAGCACACTTTCCTCGTGAAGGT
GTCTTTGTTTCAAATGGCACACACTGGTTTGTAACACAAAGGAATTTTTATGAAC
CACAAATCATTACTACAGACAACACATTTGTGTCTGGTAACTGTGATGTTGTAAT
AGGAATTGTCAACAACACAGTTTATGATCCTTTGCAACCTGAATTAGACTCATTC
AAGGAGGAGTTAGATAAATATTTTAAGAATCATACATCACCAGATGTTGATTTAG
GTGACATCTCTGGCATTAATGCTTCAGTTGTAAACATTCAAAAAGAAATTGACCG
CCTCAATGAGGTTGCCAAGAATTTAAATGAATCTCTCATCGATCTCCAAGAACTT
GGAAAGTATGAGCAGTATATAAAATGGCCATGGTACATTTGGCTAGGTTTTATAG
CTGGCTTGATTGCCATAGTAATGGTGACAATTATGCTTTGCTGTATGACCAGTTG
CTGTAGTTGTCTCAAGGGCTGTTGTTCTTGTGGATCCTGCTGCAAATTTGATGAA
GACGACTCTGAGCCAGTGCTCAAAGGAGTCAAATTACATTACACATAAACGAAC
TTATGGATTTGTTTATGAGAATCTTCACAATTGGAACTGTAACTTTGAAGCAAGG
TGAAATCAAGGATGCTACTCCTTCAGATTTTGTTCGCGCTACTGCAACGATACCG
ATACAAGCCTCACTCCCTTTCGGATGGCTTATTGTTGGCGTTGCACTTCTTGCTG
TTTTTCAGAGCGCTTCCAAAATCATAACCCTCAAAAAGAGATGGCAACTAGCACT
CTCCAAGGGTGTTCACTTTGTTTGCAACTTGCTGTTGTTGTTTGTAACAGTTTAC
TCACACCTTTTGCTCGTTGCTGCTGGCCTTGAAGCCCCTTTTCTCTATCTTTATG
CTTTAGTCTACTTCTTGCAGAGTATAAACTTTGTAAGAATAATAATGAGGCTTTGG
CTTTGCTGGAAATGCCGTTCCAAAAACCCATTACTTTATGATGCCAACTATTTTCT
TTGCTGGCATACTAATTGTTACGACTATTGTATACCTTACAATAGTGTAACTTCTT
CAATTGTCATTACTTCAGGTGATGGCACAACAAGTCCTATTTCTGAACATGACTA
CCAGATTGGTGGTTATACTGAAAAATGGGAATCTGGAGTAAAAGACTGTGTTGT
ATTACACAGTTACTTCACTTCAGACTATTACCAGCTGTACTCAACTCAATTGAGTA
CAGACACTGGTGTTGAACATGTTACCTTCTTCATCTACAATAAAATTGTTGATGA
GCCTGAAGAACATGTCCAAATTCACACAATCGACGGTTCATCCGGAGTTGTTAA
TCCAGTAATGGAACCAATTTATGATGAACCGACGACGACTACTAGCGTGCCTTT
GTAAGCACAAGCTGATGAGTACGAACTTATGTACTCATTCGTTTCGGAAGAGAC
AGGTACGTTAATAGTTAATAGCGTACTTCTTTTTCTTGCTTTCGTGGTATTCTTGC
TAGTTACACTAGCCATCCTTACTGCGCTTCGATTGTGTGCGTACTGCTGCAATAT
TGTTAACGTGAGTCTTGTAAAACCTTCTTTTTACGTTTACTCTCGTGTTAAAAATC
TGAATTCTTCTAGAGTTCCTGATCTTCTGGTCTAAACGAACTAAATATTATATTAG
TTTTTCTGTTTGGAACTTTAATTTTAGCCATGGCAGATTCCAACGGTACTATTACC
GTTGAAGAGCTTAAAAAGCTCCTTGAACAATGGAACCTAGTAATAGGTTTCCTAT
TCCTTACATGGATTTGTCTTCTACAATTTGCCTATGCCAACAGGAATAGGTTTTT
GTATATAATTAAGTTAATTTTCCTCTGGCTGTTATGGCCAGTAACTTTAGCTTGTT
TTGTGCTTGCTGCTGTTTACAGAATAAATTGGATCACCGGTGGAATTGCTATCGC
AATGGCTTGTCTTGTAGGCTTGATGTGGCTCAGCTACTTCATTGCTTCTTTCAGA
CTGTTTGCGCGTACGCGTTCCATGTGGTCATTCAATCCAGAAACTAACATTCTTC
TCAACGTGCCACTCCATGGCACTATTCTGACCAGACCGCTTCTAGAAAGTGAAC
TCGTAATCGGAGCTGTGATCCTTCGTGGACATCTTCGTATTGCTGGACACCATC
TAGGACGCTGTGACATCAAGGACCTGCCTAAAGAAATCACTGTTGCTACATCAC
GAACGCTTTCTTATTACAAATTGGGAGCTTCGCAGCGTGTAGCAGGTGACTCAG
GTTTTGCTGCATACAGTCGCTACAGGATTGGCAACTATAAATTAAACACAGACCA
TTCCAGTAGCAGTGACAATATTGCTTTGCTTGTACAGTAAGTGACAACAGATGTT
TCATCTCGTTGACTTTCAGGTTACTATAGCAGAGATATTACTAATTATTATGAGGA
CTTTTAAAGTTTCCATTTGGAATCTTGATTACATCATAAACCTCATAATTAAAAATT
TATCTAAGTCACTAACTGAGAATAAATATTCTCAATTAGATGAAGAGCAACCAAT
GGAGATTGATTAAACGAACATGAAAATTATTCTTTTCTTGGCACTGATAACACTC
GCTACTTGTGAGCTTTATCACTACCAAGAGTGTGTTAGAGGTACAACAGTACTTT
TAAAAGAACCTTGCTCTTCTGGAACATACGAGGGCAATTCACCATTTCATCCTCT
AGCTGATAACAAATTTGCACTGACTTGCTTTAGCACTCAATTTGCTTTTGCTTGTC
CTGACGGCGTAAAACACGTCTATCAGTTACGTGCCAGATCAGTTTCACCTAAAC
TGTTCATCAGACAAGAGGAAGTTCAAGAACTTTACTCTCCAATTTTTCTTATTGTT
GCGGCAATAGTGTTTATAACACTTTGCTTCACACTCAAAAGAAAGACAGAATGAT
TGAACTTTCATTAATTGACTTCTATTTGTGCTTTTTAGCCTTTCTGCTATTCCTTGT
TTTAATTATGCTTATTATCTTTTGGTTCTCACTTGAACTGCAAGATCATAATGAAA
CTTGTCACGCCTAAACGAACATGAAATTTCTTGTTTTCTTAGGAATCATCACAACT
GTAGCTGCATTTCACCAAGAATGTAGTTTACAGTCATGTACTCAACATCAACCAT
ATGTAGTTGATGACCCGTGTCCTATTCACTTCTATTCTAAATGGTATATTAGAGTA
GGAGCTAGAAAATCAGCACCTTTAATTGAATTGTGCGTGGATGAGGCTGGTTCT
AAATCACCCATTCAGTACATCGATATCGGTAATTATACAGTTTCCTGTTTACCTTT
TACAATTAATTGCCAGGAACCTAAATTGGGTAGTCTTGTAGTGCGTTGTTCGTTC
TATGAAGACTTTTTAGAGTATCATGACGTTCGTGTTGTTTTAGATTTCATCTAAAC
GAACAAACTAAAATGTCTGATAATGGACCCCAAAATCAGCGAAATGCACCCCGC
ATTACGTTTGGTGGACCCTCAGATTCAACTGGCAGTAACCAGAATGGAGAACGC
AGTGGGGCGCGATCAAAACAACGTCGGCCCCAAGGTTTACCCAATAATACTGC
GTCTTGGTTCACCGCTCTCACTCAACATGGCAAGGAAGACCTTAAATTCCCTCG
AGGACAAGGCGTTCCAATTAACACCAATAGCAGTCCAGATGACCAAATTGGCTA
CTACCGAAGAGCTACCAGACGAATTCGTGGTGGTGACGGTAAAATGAAAGATCT
CAGTCCAAGATGGTATTTCTACTACCTAGGAACTGGGCCAGAAGCTGGACTTCC
CTATGGTGCTAACAAAGACGGCATCATATGGGTTGCAACTGAGGGAGCCTTGAA
TACACCAAAAGATCACATTGGCACCCGCAATCCTGCTAACAATGCTGCAATCGT
GCTACAACTTCCTCAAGGAACAACATTGCCAAAAGGCTTCTACGCAGAAGGGAG
CAGAGGCGGCAGTCAAGCCTCTTCTCGTTCCTCATCACGTAGTCGCAACAGTTC
AAGAAATTCAACTCCAGGCAGCAGTAGGGGAACTTCTCCTGCTAGAATGGCTGG
CAATGGCGGTGATGCTGCTCTTGCTTTGCTGCTGCTTGACAGATTGAACCAGCT
TGAGAGCAAAATGTCTGGTAAAGGCCAACAACAACAAGGCCAAACTGTCACTAA
GAAATCTGCTGCTGAGGCTTCTAAGAAGCCTCGGCAAAAACGTACTGCCACTAA
AGCATACAATGTAACACAAGCTTTCGGCAGACGTGGTCCAGAACAAACCCAAGG
AAATTTTGGGGACCAGGAACTAATCAGACAAGGAACTGATTACAAACATTGGCC
GCAAATTGCACAATTTGCCCCCAGCGCTTCAGCGTTCTTCGGAATGTCGCGCAT
TGGCATGGAAGTCACACCTTCGGGAACGTGGTTGACCTACACAGGTGCCATCA
AATTGGATGACAAAGATCCAAATTTCAAAGATCAAGTCATTTTGCTGAATAAGCA
TATTGACGCATACAAAACATTCCCACCAACAGAGCCTAAAAAGGACAAAAAGAA
GAAGGCTGATGAAACTCAAGCCTTACCGCAGAGACAGAAGAAACAGCAAACTGT
GACTCTTCTTCCTGCTGCAGATTTGGATGATTTCTCCAAACAATTGCAACAATCC
ATGAGCAGTGCTGACTCAACTCAGGCCTAAACTCATGCAGACCACACAAGGCAG
ATGGGCTATATAAACGTTTTCGCTTTTCCGTTTACGATATATAGTCTACTCTTGTG
CAGAATGAATTCTCGTAACTACATAGCACAAGTAGATGTAGTTAACTTTAATCTC
ACATAGCAATCTTTAATCAGTGTGTAACATTAGGGAGGACTTGAAAGAGCCACC
ACATTTTCACCGAGGCCACGCGGAGTACGATCGAGTGTACAGTGAACAATGCTA
GGGAGAGCTGCCTATATGGAAGAGCCCTAATGTGTAAAATTAATTTTAGTAGTG
CTATCCCCATGTGATTTTAATAGCTTCTTAGGAGAATGACAAAAAAAAAAAAAAAA
AAAAAAAAAAAAAAAAA
SEQ ID NO: 14, which is 30-44 of SEQ ID NO: 1
SSVLHSTQDLFLPFF
SEQ ID NO: 15, which is 48-68 of SEQ ID NO: 1
TWFHAIHVSGTNGTKR
SEQ ID NO: 16, which is 110-166 of SEQ ID NO: 1
NVVIKVCEFQFCNDPFLGVYYHKNNKSWMESEFRVYSSANNCTFEYVSQPFLMDL
EG
SEQ ID NO: 17, which is 200-214 of SEQ ID NO: 1
DLPQGFSALEPLVDL
SEQ ID NO: 18, which is 226-250 of SEQ ID NO: 1
LLALHRSYLTPGDSSSGWTAGAAAY
SEQ ID NO: 19, which is 256-277 of SEQ ID NO: 1
QPRTFLLKYNENGTITDAVDCALDP
SEQ ID NO: 20, which is 328-342 of SEQ ID NO: 1
NATRFASVYAWNRKR
SEQ ID NO: 21, which is 399-414 of SEQ ID NO: 1
TGKIADYNYKLPDDF
SEQ ID NO: 22, which is 434-448 of SEQ ID NO: 1
YNYLYRLFRKSNLKP
SEQ ID NO: 23, which is 550-572 of SEQ ID NO: 1
FGRDIADTTDAVRDPQTLEILDI
SEQ ID NO: 24, which is 590-604 of SEQ ID NO: 1
SNQVAVLYQDVNCTE
SEQ ID NO: 25, which is 632-650 of SEQ ID NO: 1
AGCLIGAEHVNNSYECDIP
SEQ ID NO: 26, which is 354-368 of SEQ ID NO: 1
YNSASFSTFKCYGVS
SEQ ID NO: 27, which is 622-636 of SEQ ID NO: 1
STGSNVFQTRAGCLI
SEQ ID NO: 28, which is 30-44 of SEQ ID NO: 1
SSVLHSTQDLFLPFF
SEQ ID NO: 29, which is 226-250 of SEQ ID NO: 1
LLALHRSYLTPGDSSSGWTAGAAAY
SEQ ID NO: 30: SARS-CoV-2 N protein
MSDNGPQNQRNAPRITFGGPSDSTGSNQNGERSGARSKQRRPQGLPNNTASWFT
ALTQHGKEDLKFPRGQGVPINTNSSPDDQIGYYRRATRRIRGGDGKMKDLSPRWY
FYYLGTGPEAGLPYGANKDGIIWVATEGALNTPKDHIGTRNPANNAAIVLQLPQGTT
LPKGFYAEGSRGGSQASSRSSSRSRNSSRNSTPGSSRGTSPARMAGNGGDAALA
LLLLDRLNQLESKMSGKGQQQQGQTVTKKSAAEASKKPRQKRTATKAYNVTQAFG
RRGPEQTQGNFGDQELIRQGTDYKHWPQIAQFAPSASAFFGMSRIGMEVTPSGTW
LTYTGAIKLDDKDPNFKDQVILLNKHIDAYKTFPPTEPKKDKKKKADETQALPQRQKK
QQTVTLLPAADLDDFSKQLQQSMSSADSTQA
SEQ ID NO: 31: RBD, a fragment from S1 domain from
SARS-CoV-2 S protein
RVQPTESIVRFPNITNLCPFGEVFNATRFASVYAWNRKRISNCVADYSVLYNSASFS
TFKCYGVSPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNGVEGFNC
YFPLQSYGFQPTNGVGYQPYRVVVLSFELLHAPATVCGPKKSTNLVKNKCVNF
SEQ ID NO: 32: RBD a fragment from S1 domain from
SARS-CoV-2 S protein, with C-terminal His tag
RVQPTESIVRFPNITNLCPFGEVFNATRFASVYAWNRKRISNCVADYSVLYNSASFS
TFKCYGVSPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNGVEGFNC
YFPLQSYGFQPTNGVGYQPYRVVVLSFELLHAPATVCGPKKSTNLVKNKCVNFLEH
HHHHHHH
SEQ ID NO: 33: S2 domain from SARS-CoV-2 S protein
SVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFN
FSQILPDPSKPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIGVTQNVLY
ENQKLIANQFNSAIGKIQDSLSSTASALGKLQDVVNQNAQALNTLVKQLSSNFGAISS
VLNDILSRLDKVEAEVQIDRLITGRLQSLQTYVTQQLIRAAEIRASANLAATKMSECVL
GQSKRVDFCGKGYHLMSFPQSAPHGVVFLHVTYVPAQEKNFTTAPAICHDGKAHF
PREGVFVSNGTHWFVTQRNFYEPQIITTDNTFVSGNCDVVIGIVNNTVYDPLQPELD
SFKEELDKYFKNHTSPDVDLGDISGINASVVNIQKEIDRLNEVAKNLNESLIDLQELGK
YEQYIKWPWYIWLGFIAGLIAIVMVTIMLCCMTSCCSCLKGCCSCGSCCKFDEDDSE
PVLKGVKLHYT
SEQ ID NO: 34: RBD as used in the examples
MKHLWFFLLLVAAPRWVLSGPMRVQPTESIVRFPNITNLCPFGEVFNATRFASVYA
WNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSNLKPF
ERDISTEIYQAGSTPCNGVEGFNCYFPLQSYGFQPTNGVGYQPYRVVVLSFELLHA
PATVCGPKKSTNLVKNKCVNFLEHHHHHHHH
SEQ ID NO: 35 (SEQ ID NO: 1-derived peptide reactive with
SARS-CoV-2 antibodies)
LTPGDSSSGWTAG
SEQ ID NO: 36 (SEQ ID NO: 1-derived peptide reactive with
SARS-CoV-2 antibodies)
YQAGSTPCNGV
SEQ ID NO: 37 (SEQ ID NO: 1-derived peptide reactive with
SARS-CoV-2 antibodies)
YGFQPTNGVGYQ
SEQ ID NO: 38: His-tagged RBD
MKHLWFFLLLVAAPRWVLSGPMRVQPTESIVRFPNITNLCPFGEVFNATRFASVYA
WNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSNLKPF
ERDISTEIYQAGSTPCNGVEGFNCYFPLQSYGFQPTNGVGYQPYRVVVLSFELLHA
PATVCGPKKSTNLVKNKCVNFLEHHHHHHHH
SEQ ID NO: 39: human Angiotensin-converting enzyme 2 (ACE2)
MSSSSWLLLSLVAVTAAQSTIEEQAKTFLDKFNHEAEDLFYQSSLASWNYNTNITEE
NVQNMNNAGDKWSAFLKEQSTLAQMYPLQEIQNLTVKLQLQALQQNGSSVLSEDK
SKRLNTILNTMSTIYSTGKVCNPDNPQECLLLEPGLNEIMANSLDYNERLWAWESW
RSEVGKQLRPLYEEYVVLKNEMARANHYEDYGDYWRGDYEVNGVDGYDYSRGQL
IEDVEHTFEEIKPLYEHLHAYVRAKLMNAYPSYISPIGCLPAHLLGDMWGRFWTNLY
SLTVPFGQKPNIDVTDAMVDQAWDAQRIFKEAEKFFVSVGLPNMTQGFWENSMLT
DPGNVQKAVCHPTAWDLGKGDFRILMCTKVTMDDFLTAHHEMGHIQYDMAYAAQP
FLLRNGANEGFHEAVGEIMSLSAATPKHLKSIGLLSPDFQEDNETEINFLLKQALTIV
GTLPFTYMLEKWRWMVFKGEIPKDQWMKKWWEMKREIVGVVEPVPHDETYCDPA
SLFHVSNDYSFIRYYTRTLYQFQFQEALCQAAKHEGPLHKCDISNSTEAGQKLFNML
RLGKSEPWTLALENVVGAKNMNVRPLLNYFEPLFTWLKDQNKNSFVGWSTDWSP
YADQSIKVRISLKSALGDKAYEWNDNEMYLFRSSVAYAMRQYFLKVKNQMILFGEE
DVRVANLKPRISFNFFVTAPKNVSDIIPRTEVEKAIRMSRSRINDAFRLNDNSLEFLGI
QPTLGPPNQPPVSIWLIVFGVVMGVIVVGIVILIFTGIRDRKKKNKARSGENPYASIDIS
KGENNPGFQNTDDVQTSF
SEQ ID NO: 40 (SEQ ID NO: 1-derived peptide reactive with
SARS-CoV-2 antibodies)
RTWLPPAYTNS
SEQ ID NO: 41 (SEQ ID NO: 1-derived peptide reactive with
SARS-CoV-2 antibodies)
RTQLPPAYTNS
SEQ ID NO: 42 (SEQ ID NO: 1-derived peptide reactive with
SARS-CoV-2 antibodies)
SGTNGTKRFDN
SEQ ID NO: 43 (SEQ ID NO: 1-derived peptide reactive with
SARS-CoV-2 antibodies)
MSHHHHHHHHSPMYSIITPNILRLESEETMVLEAHDAQGDVPVTVTVHDFPGKKLVL
SSEKTVLTPATNHMGNVTFTIPANREFKSEKGRNKFVTVQATFGTQVVEKVVLVSL
QSGIEGRMRTQLPPAYTNSRTQLPPAYTNS
SEQ ID NO: 44 (SEQ ID NO: 1-derived peptide reactive with
SARS-CoV-2 antibodies with GST fusion)
MSHHHHHHHHSPMYSIITPNILRLESEETMVLEAHDAQGDVPVTVTVHDFPGKKLVL
SSEKTVLTPATNHMGNVTFTIPANREFKSEKGRNKFVTVQATFGTQVVEKVVLVSL
QSGIEGRMMSHHHHHHHHSPMYSIITPNILRLESEETMVLEAHDAQGDVPVTVTVH
DFPGKKLVLSSEKTVLTPATNHMGNVTFTIPANREFKSEKGRNKFVTVQATFGTQV
VEKVVLVSLQSGIEGRMSGTNGTKRFDNSGTNGTKRFDN
SEQ ID NO: 45 (SEQ ID NO: 1-derived peptide reactive with
SARS-CoV-2 antibodies with GST fusion)
MSHHHHHHHHSPMYSIITPNILRLESEETMVLEAHDAQGDVPVTVTVHDFPGKKLVL
SSEKTVLTPATNHMGNVTFTIPANREFKSEKGRNKFVTVQATFGTQVVEKVVLVSL
QSGIEGRMLTPGDSSSGWTAGLTPGDSSSGWTAG
SEQ ID NO: 46 (SEQ ID NO: 1-derived peptide reactive with
SARS-CoV-2 antibodies with GST fusion)
MSHHHHHHHHSPMYSIITPNILRLESEETMVLEAHDAQGDVPVTVTVHDFPGKKLVL
SSEKTVLTPATNHMGNVTFTIPANREFKSEKGRNKFVTVQATFGTQVVEKVVLVSL
QSGIEGRMNNLDSKVGGNNLDSKVGG
SEQ ID NO: 47 (SEQ ID NO: 1-derived peptide reactive with
SARS-CoV-2 antibodies with GST fusion)
MSHHHHHHHHSPMYSIITPNILRLESEETMVLEAHDAQGDVPVTVTVHDFPGKKLVL
SSEKTVLTPATNHMGNVTFTIPANREFKSEKGRNKFVTVQATFGTQVVEKVVLVSL
QSGIEGRMYQAGSTPCNGVYQAGSTPCNGV
SEQ ID NO: 48 (SEQ ID NO: 1-derived peptide reactive with
SARS-CoV-2 antibodies with GST fusion)
MSHHHHHHHHSPMYSIITPNILRLESEETMVLEAHDAQGDVPVTVTVHDFPGKKLVL
SSEKTVLTPATNHMGNVTFTIPANREFKSEKGRNKFVTVQATFGTQVVEKVVLVSL
QSGIEGRMYGFQPTNGVGYQYGFQPTNGVGYQ
SEQ ID NO: 49 (SEQ ID NO: 1-derived peptide reactive with
SARS-CoV-2 antibodies with GST fusion, P1-P6)
MSHHHHHHHHSPMYSIITPNILRLESEETMVLEAHDAQGDVPVTVTVHDFPGKKLVL
SSEKTVLTPATNHMGNVTFTIPANREFKSEKGRNKFVTVQATFGTQVVEKVVLVSL
QSGIEGRMRTQLPPAYTNSSGTNGTKRFDNLTPGDSSSGWTAGNNLDSKVGGYQ
AGSTPCNGVYGFQPTNGVGYQ
SEQ ID NO: 50 (C-terminally His-tagged extracellular domain
of human ACE2)
MSSSSWLLLSLVAVTAAQSTIEEQAKTFLDKFNHEAEDLFYQSSLASWNYNTNITEE
NVQNMNNAGDKWSAFLKEQSTLAQMYPLQEIQNLTVKLQLQALQQNGSSVLSEDK
SKRLNTILNTMSTIYSTGKVCNPDNPQECLLLEPGLNEIMANSLDYNERLWAWESW
RSEVGKQLRPLYEEYVVLKNEMARANHYEDYGDYWRGDYEVNGVDGYDYSRGQL
IEDVEHTFEEIKPLYEHLHAYVRAKLMNAYPSYISPIGCLPAHLLGDMWGRFWTNLY
SLTVPFGQKPNIDVTDAMVDQAWDAQRIFKEAEKFFVSVGLPNMTQGFWENSMLT
DPGNVQKAVCHPTAWDLGKGDFRILMCTKVTMDDFLTAHHEMGHIQYDMAYAAQP
FLLRNGANEGFHEAVGEIMSLSAATPKHLKSIGLLSPDFQEDNETEINFLLKQALTIV
GTLPFTYMLEKWRWMVFKGEIPKDQWMKKWWEMKREIVGVVEPVPHDETYCDPA
SLFHVSNDYSFIRYYTRTLYQFQFQEALCQAAKHEGPLHKCDISNSTEAGQKLFNML
RLGKSEPWTLALENVVGAKNMNVRPLLNYFEPLFTWLKDQNKNSFVGWSTDWSP
YADQSIKVRISLKSALGDKAYEWNDNEMYLFRSSVAYAMRQYFLKVKNQMILFGEE
DVRVANLKPRISFNFFVTAPKNVSDIIPRTEVEKAIRMSRSRINDAFRLNDNSLEFLGI
QPTLGPPNQPPVSLEGSGSGSHHHHHHHHGSGLNDIFEAQKIEWHE
SEQ ID NO: 51 (SEQ ID NO: 1 with mutations of SARS-CoV-2
U.K. variant B.1.1.7
VNLTTRTQLPPAYTNSFTRGVYYPDKVFRSSVLHSTQDLFLPFFSNVTWFHAISGTN
GTKRFDNPVLPFNDGVYFASTEKSNIIRGWIFGTTLDSKTQSLLIVNNATNVVIKVCE
FQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHR
SYLTPGDSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGK
IADYNYKLPDDFTGCVIAWNSNNLDSKVGGNYNYLYRLFRKSNLKPFERDISTEIYQ
AGSTPCNGVEGFNCYFPLQSYGFQPTYGVGYQPYRVVVLSFELLHAPATVCGPKK
STNLVKNKCVNFNFNGLTGTGVLTESNKKFLPFQQFGRDIDDTTDAVRDPQTLEILD
TPCSFGGVSVITPGTNTSNQVAVLYQGVNCTEVPVAIHADQLTPTWRVYSTGSNVF
QTRAGCLIGAEHVNNSYECDIH
SEQ ID NO: 52 (SEQ ID NO: 1 with mutations of SARS-CoV-2
South African variant B.1.351):
VNLTTRTQLPPAYTNSFTRGVYYPDKVFRSSVLHSTQDLFLPFFSNVTWFHAIHVSG
TNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRI
SNCVADYSVLYNSASFSTFKCYGVSPTKLNDLCFTNVYADSFVIRGDEVRQIAPGQT
GNIADYNYKLYDDFTGCVIAWNSNNLDSKVGGNYNYLYRLFRKSNLKPFERDISTEI
YQAGSTPCNGVKGFNCYFPLQSYGFQPTYGVGYQPYRVVVLSFELLHAPATVCGP
KKSTNLVKNKCVNFNFNGLTGTGVLTESNKKFLPFQQFGRDIADTTDAVRDPQTLEI
LDITPCSFGGVSVITPGTNTSNQVAVLYQGVNCTEVPVAIHADQLTPTWRVYSTGSN
VFQTRAGCLIGAEHVNNSYECDIP
SEQ ID NO: 53 (SEQ ID NO: 1 with mutations of SARS-CoV-2
Brazilian variant P.1):
VNLTTRTQLPPAYTNSFTRGVYYPDKVFRSSVLHSTQDLFLPFFSNVTWFHAIHVSG
TNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRI
SNCVADYSVLYNSASFSTFKCYGVSPTKLNDLCFTNVYADSFVIRGDEVRQIAPGQT
GKIADYNYKLPDDFTGCVIAWNSNNLDSKVGGNYNYLYRLFRKSNLKPFERDISTEI
YQAGSTPCNGVKGFNCYFPLQSYGFQPTYGVGYQPYRVVVLSFELLHAPATVCGP
KKSTNLVKNKCVNFNFNGLTGTGVLTESNKKFLPFQQFGRDIADTTDAVRDPQTLEI
LDITPCSFGGVSVITPGTNTSNQVAVLYQDVNCTEVPVAIHADQLTPTWRVYSTGSN
VFQTRAGCLIGAEHVNNSYECDIP
SEQ ID NO: 54 (SEQ ID NO: 1 with mutations of SARS-CoV-2
Mink Variant from Denmark):
VNLTTRTQLPPAYTNSFTRGVYYPDKVFRSSVLHSTQDLFLPFFSNVTWFHAISGTN
GTKRFDNPVLPFNDGVYFASTEKSNIIRGWIFGTTLDSKTQSLLIVNNATNVVIKVCE
FQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHR
SYLTPGDSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGK
IADYNYKLPDDFTGCVIAWNSNNLDSKVGGNYNYLFRLFRKSNLKPFERDISTEIYQ
AGSTPCNGVEGFNCYFPLQSYGFQPTNGVGYQPYRVVVLSFELLHAPATVCGPKK
STNLVKNKCVNFNFNGLTGTGVLTESNKKFLPFQQFGRDIADTTDAVRDPQTLEILDI
TPCSFGGVSVITPGTNTSNQVAVLYQGVNCTEVPVAIHADQLTPTWRVYSTGSNVF
QTRAGCLIGAEHVNNSYECDIP
SEQ ID NO: 55 (SEQ ID NO: 1-derived peptide reactive with
SARS-CoV-2 antibodies)
NNLDSKVGG
SEQ ID NO: 56 (SEQ ID NO: 1-derived peptide reactive with
SARS-CoV-2 antibodies with GST fusion, P1-P6)
RTQLPPAYTNSSGTNGTKRFDNLTPGDSSSGWTAGNNLDSKVGGYQAGSTPCNG
VYGFQPTNGVGYQ

The present invention is further illustrated by the following examples, sequences and figures from which further features, embodiments, aspects and advantages of the present invention may be taken. All methods and materials similar or equivalent to those described herein can be used in the practice or testing of the present invention, with suitable methods and materials being described herein. All publications, patent applications, patents, and other references mentioned herein are incorporated by reference in their entirety.

### EXAMPLES

### Example 1: Detection of Antibodies to SEQ ID NO: 1 Using an ELISA Immunoassay

Samples

Eight samples from patients tested SARS-CoV-2 positive by FOR as described by Corman et al. (Corman et al. (2020) Diagnostic detection of 2019-nCoV by real-time RT-PCR, www.who.int/docs/default-source/coronaviruse/protocol-v2-1.pdf?sfvrsn=a9ef618c_2) were obtained 6 to 14 days after the infection and 14 samples from such patients obtained at an earlier time point after the infection were available.

In addition, a range of samples containing various coronaviruses was available, including 18 samples from patients infected with MERS, three samples from patients infected with SARS-CoV-1, four patients with NL63, three patients with 229E, six patients with 0043 and three patients with HKU1.

Preparation of microtiter plates coated with antigen: SEQ ID NO: 2 was expressed in HEK293T cells using standard cloning of SEQ ID NO: 4 into the pTriEx-1 plasmid with an artificial signal sequence and a C-terminal His tag, resulting in the expression of SEQ ID NO: 2 and, after removal of the signal peptide, SEQ ID NO: 3. Transfected cells were cultured at 37° C. and 8.5% CO₂ in Dulbecco's modified eagle's medium with 10% fetal calf serum, 100 U/ml penicillin and 0.1 mg/ml streptomycin for three to five days. Cells were harvested, resuspended in 20 mM Tris-HCl pH 7.4, 10% (w/v) sucrose, 5 mM EDTA, 1 mM PMSF and stored at −80° C. until further use.

To prepare SEQ ID NO: 3, cell culture supernatant was adjusted to 5 mmol/l tris chloride pH 8.0, 164 mmol/l sodium chloride, 50 mmol/I magnesium chloride, 20 mmol/limidazole, 0,1% Triton X-100, cleared by centrifugation for 30 minutes at 17,600×g, 4° C., applied to Nickel Rapid Run (Agarose Bead Technologies, Miami, Fla., USA) equilibrated with 5 mmol/l tris chloride pH 8.0, 300 mmol/l sodium chloride, 20 mmol/l imidazole and eluted by increasing the imidazole concentration to 150 mmol/l.

All fractions containing SEQ ID NO: 3 were pooled and concentrated by ultrafiltration (VivaSpin, Sartorius, Gottingen, Germany). The final preparation was stored at −80° C. until further use.

The final protein preparation of SEQ ID NO: 3 was treated with or without 16 mmol/l dithiothreitol and incubated at 70° C. or at room temperature for 10 minutes, followed by SDS gel electrophoresis and Coomassie staining. Protein identity was verified by mass spectrometry.

For use in microtiter ELISA the purified protein was diluted in PBS to final concentrations of approximately 1.5 μg/ml and used to coat ELISA microtiter plates (Nunc, Roskilde, Denmark) overnight.

Experimental procedure: Samples were diluted 1:101 in IgG sample buffer, applied to microtiter plates and incubated as described for commercial EUROIMMUN ELISA Test-Kits, using reagents commercially available (e.g. El 2260-9601 G/A, which is a buffer having essentially physiological conditions regarding salt concentration and pH). The manual of El 2260-9601 G/A was followed. In brief: 60 min at 37° C.; 3 washing steps using washing buffer; addition of 100 μl of peroxidase-labelled anti-human IgG conjugate (rabbit) or anti-human IgA conjugate (rabbit) per well; incubation for 30 min at 37° C.; 3 washing steps using EUROIMMUN washing buffer; addition of 100 μl of chromogen/substrate solution (TMB/H202) per well; incubation for 30 min at room temperature; addition of 100 μl stop-solution (0.5 M sulfuric acid); measurement of optical density at 450 nm against 630 nm as a reference.

Calibration was carried out using commercially available calibrators (product number El 2606-9601 A, EUROIMMUN Medizinische Labordiagnostika AG). A ratio was calculated by dividing extinction of the control or patient sample by the extinction of the calibrator. Results below 0.8 were considered negative, results between 0.8 and 1.1 borderline, and results of more than 1.1 positive.

Results: The primary data are shown in Table 1:

| | | | | Cut-Off | | | Cut-Off |
|---|---|---|---|---|---|---|---|
| | | | | OD | | | OD |
| | | | | 0.100 | | | 0.200 |
| | | | | IgG | | | IgA |
| | | | | (Ratio) | | | (Ratio) |
| | | | | pos: | | | pos: |
| | | raw data | | ≥1.1 | raw data | | ≥1.1 |
| | | IgG | | bl: | IgA | | bl: |
| | | (OD) | dil. 1: | 0.8-1.0 | (OD) | dil. 1: | 0.8-1.0 |
| 1 | Calibrator | 1.911 | 300 | 19.1 | 3.055 | 600 | 15.3 |
| 2 | Calibrator | 1.593 | 600 | 15.9 | 2.204 | 1200 | 11.0 |
| 3 | Calibrator | 1.077 | 1200 | 10.8 | 1.068 | 2400 | 5.3 |
| 4 | Calibrator | 0.697 | 2400 | 7.0 | 0.529 | 4800 | 2.6 |
| 5 | Calibrator | 0.441 | 4800 | 4.4 | 0.314 | 9600 | 1.6 |
| 6 | Calibrator | 0.248 | 9600 | 2.5 | 0.155 | 19200 | 0.8 |
| 7 | Calibrator | 0.132 | 19200 | 1.3 | 0.078 | 38400 | 0.4 |
| 8 | Calibrator | 0.066 | 38400 | 0.7 | 0.051 | 76800 | 0.3 |
| 9 | SARS-CoV-2* | 0.157 | | 1.6 | 1.402 | | 7.0 |
| 10 | SARS-CoV-2* | 0.075 | | 0.8 | 0.377 | | 1.9 |
| 11 | SARS-CoV-2* | 0.276 | | 2.8 | 9.999 | | 50.0 |
| 12 | SARS-CoV-2* | 0.027 | | 0.3 | 0.027 | | 0.1 |
| 13 | SARS-CoV-2* | 0.023 | | 0.2 | 0.064 | | 0.3 |
| 14 | SARS-CoV-2* | 0.119 | | 1.2 | 1.142 | | 5.7 |
| 15 | SARS-CoV-2* | 0.031 | | 0.3 | 0.203 | | 1.0 |
| 16 | SARS-CoV-2* | 0.079 | | 0.8 | 0.385 | | 1.9 |
| 17 | SARS-COV-2** | 0.021 | | 0.2 | 0.055 | | 0.3 |
| 18 | SARS-COV-2** | 0.019 | | 0.2 | 0.084 | | 0.4 |
| 19 | SARS-COV-2** | 0.021 | | 0.2 | 0.225 | | 1.1 |
| 20 | SARS-COV-2** | 0.018 | | 0.2 | 0.192 | | 1.0 |
| 21 | SARS-COV-2** | 0.033 | | 0.3 | 0.599 | | 3.0 |
| 22 | SARS-COV-2** | 0.029 | | 0.3 | 0.331 | | 1.7 |
| 23 | SARS-COV-2** | 0.013 | | 0.1 | 0.030 | | 0.2 |
| 24 | SARS-COV-2** | 0.017 | | 0.2 | 0.097 | | 0.5 |
| 25 | SARS-COV-2** | 0.027 | | 0.3 | 0.214 | | 1.1 |
| 26 | SARS-COV-2** | 0.016 | | 0.2 | 0.021 | | 0.1 |
| 27 | SARS-COV-2** | 0.014 | | 0.1 | 0.073 | | 0.4 |
| 28 | SARS-COV-2** | 0.014 | | 0.1 | 0.042 | | 0.2 |
| 29 | SARS-COV-2** | 0.015 | | 0.2 | 0.030 | | 0.2 |
| 30 | SARS-COV-2** | 0.015 | | 0.2 | 0.079 | | 0.4 |
| 31 | MERS 1 | 0.013 | | 0.1 | 0.042 | | 0.2 |
| 32 | MERS 2 | 0.008 | | 0.1 | 0.017 | | 0.1 |
| 33 | MERS 3 | 0.011 | | 0.1 | 0.038 | | 0.2 |
| 34 | MERS 4 | 0.008 | | 0.1 | 0.011 | | 0.1 |
| 35 | MERS 5 | 0.008 | | 0.1 | 0.027 | | 0.1 |
| 36 | MERS 6 | 0.008 | | 0.1 | 0.035 | | 0.2 |
| 37 | MERS 7 | 0.009 | | 0.1 | 0.013 | | 0.1 |
| 38 | MERS 8 | 0.017 | | 0.2 | 0.036 | | 0.2 |
| 39 | MERS 9 | 0.010 | | 0.1 | 0.024 | | 0.1 |
| 40 | MERS 10 | 0.007 | | 0.1 | 0.012 | | 0.1 |
| 41 | MERS 11 | 0.020 | | 0.2 | 0.026 | | 0.1 |
| 42 | MERS 12 | 0.008 | | 0.1 | 0.026 | | 0.1 |
| 43 | MERS 13 | 0.015 | | 0.2 | 0.021 | | 0.1 |
| 44 | MERS 14 | 0.012 | | 0.1 | 0.036 | | 0.2 |
| 45 | MERS 15 | 0.024 | | 0.2 | 0.066 | | 0.3 |
| 46 | MERS 16 | 0.021 | | 0.2 | 0.080 | | 0.4 |
| 47 | MERS 17 | 0.008 | | 0.1 | 0.039 | | 0.2 |
| 48 | MERS 18 | 0.029 | | 0.3 | 0.104 | | 0.5 |
| 49 | SARS-1 | 0.214 | | 2.1 | 0.285 | | 1.4 |
| 50 | SARS-1 | 0.596 | | 6.0 | 0.227 | | 1.1 |
| 51 | SARS-1 | 0.128 | | 1.3 | 0.260 | | 1.3 |
| 52 | OC43 | 0.035 | | 0.4 | 0.126 | | 0.6 |
| 53 | OC43 | 0.029 | | 0.3 | 0.098 | | 0.5 |
| 54 | OC43 | 0.016 | | 0.2 | 0.041 | | 0.2 |
| 55 | OC43 | 0.011 | | 0.1 | 0.048 | | 0.2 |
| 68 | NL63 | 0.013 | | 0.1 | 0.023 | | 0.1 |
| 69 | NL63 | 0.027 | | 0.3 | 0.039 | | 0.2 |
| 70 | NL63 | 0.036 | | 0.4 | 0.057 | | 0.3 |
| 71 | NL63 | 0.019 | | 0.2 | 0.030 | | 0.2 |
| 72 | 229E | 0.041 | | 0.4 | 0.045 | | 0.2 |
| 73 | 229E | 0.022 | | 0.2 | 0.024 | | 0.2 |
| 74 | 229E | 0.030 | | 0.3 | 0.034 | | 0.2 |
| 75 | OC43 | 0.050 | | 0.5 | 0.100 | | 0.5 |
| 76 | OC43 | 0.079 | | 0.8 | 0.201 | | 1.0 |
| 77 | HKU1 | 0.023 | | 0.2 | 0.054 | | 0.3 |
| 78 | HKU1 | 0.019 | | 0.2 | 0.055 | | 0.3 |
| 79 | HKU1 | 0.010 | | 0.1 | 0.029 | | 0.1 |

(*late stage; **<day 6)

Conclusions

The results show that antibodies to SEQ 10 NO: 1 may be used to diagnose a SARS-CoV-2 infection in samples from human patients.

Comparison of the data obtained with secondary antibodies recognizing IgG and IgA class antibodies shows that the detection of IgA antibodies is more sensitive, at least at an early stage of the disease: 4/14 patient samples taken at an earlier stage of the infection, before six days post onset of illness, could be correctly identified as positive when IgA class antibodies were detected, while the detection of IgG in the same samples gave negative results.

Both assays showed cross-reactivity with samples from SARS-CoV-1 patients, but virtually none of the samples from patients infected with MERS, NL63, 229E, OC43 and HKU1. Distinction between SARS-CoV-1 and SARS-CoV-2 is possible based on the different time-resolved Ig class signature, in particular the later emergence of IgA class antibodies in SARS-CoV-1 (Hsue, P. R., Huang, L. M., Chen, P. J., Kao, C. L., and Yang P. C. (2004) Chronological evolution of IgM, IgA, IgG and neutralization antibodies after infection with SARS-associated coronavirus, Clinical Microbiology and Infection, 10(12), 1062-1066). Not in the least, hardly any cases of SARS-CoV have been reported since the outbreak of SARS-CoV-2.

Various post published publications by independent researchers confirmed the inventors' findings:

Jääskelainen et al. concluded from a comparative study with six commercially available serological assays for detection of SARS-CoV-2 IgG, IgA and/or IgM antibodies that, among the six assays tested, the EUROIMMUN assay based on the detection of IgA to S1 provided the highest sensitivity (Jaaskelainen, A. J., Kuivanen, S., Keklainen, E., Ahava, M. J., Loginov, R., Kallio-Kokko, H., Vapalahti, O., Jarva, H., Kurkela, and Lappalainen, M. (2020), J. Olin. Virology 129, 104512). Okba et al. obtained similar results. (Okba et al., Severe Acute Respiratory Syndrome Coronavirus 2-Specific Antibody Responses in Coronavirus Disease Patients, Emerg Infect Dis. 2020 July; 26(7):1478-1488, prepublished on medRxiv 2020.03.18.20038059).Beavis et al. confirmed that the sensitivity of the IgA-based assay according to the present invention is superior at an early stage of the disease, while the IgG assay is superior at a later stage (Beavis, K. G., Mathushek, S. M., Abeleda, A. P. F., Bethel, C., Hunt, C., Gillen, S., Moran, A., and Tesic, V. (2020) Evalutaion of the EUROIMMUN Anti-SARS-CoV-2 ELISA Assay for detection of IgA and IgG antibodies, J. Olin. Virology 129, 104468).

In summary, the assay according to the present invention may be used for the early detection of diagnostically relevant antibodies to SECS ID NO: 1. In some patients, true positive results can be found before six days have passed since the onset of symptoms. Therefore, the assay helps dose or at least narrow down the diagnostic gap between the period when PCR-based assays and immunoassays may be used.

### Example 2: An Extended ELISA Study Aiming to Further Characterize the Diagnostic Reliability and Relevance of Tests for the Detection of IgA Antibodies to SEQ ID NO: 1

For the purpose of determining the sensitivity of the instant assay, the presence or absence of IgA antibodies was detected in 166 samples from 152 European patients using EUROIMMUN product no. El 2606-9601 A, based on an assay similar as described in Example 1. In each of the subject samples, a SARS-CoV-2 infection had been confirmed using RT-PCR according to Gorman V M, et al. Detection of 2019 novel coronavirus (2019-nCoV) by real-time RT-PCR. Euro Surveill 25(3): pii=2000045 (2020-01-23) based on a sample from an early stage of the infection.

Briefly, this assay is based on an ELISA using a microtiter plate coated with an antigen comprising purified SEQ ID NO: 1. Following incubation with samples and extensive washing steps using physiological buffers, a secondary antibody to IgA labeled with an enzymatically active label is used to specifically decorate IgA antibodies to SEQ ID NO: 1, followed by incubation of a chromogenic substrate. Further details can be found in the manual supplied with the product (EUROIMMUN product no. El 2606-9601 A), which product manual is herewith incorporated by reference.

The serological characterization was carried out based on samples obtained in the following course of the infection.

A sensitivity of 60.2% regarding IgA to SEQ ID NO: 1 was determined with samples obtained until day 10 post onset of symptoms (or obtained until day 10 after direct detection of infection by positive RT-PCR). The sensitivity was 98.6% for samples obtained after day 10.

The results and immunoresponses are highly individual though. For example, a minority of patients may not show IgA, IgG or IgM responses at all.

Taken together, this further study also consistently demonstrates that the specific detection of IgA class antibodies directed against SEQ ID NO: 1 provides particularly high sensitivity for detecting SARS-CoV-2 infection already at an early stage of disease.

### Example 3: Persistence of Various Diagnostically Relevant Antibodies Over Time in SARS-CoV-2 Patients as Shown by ELISA

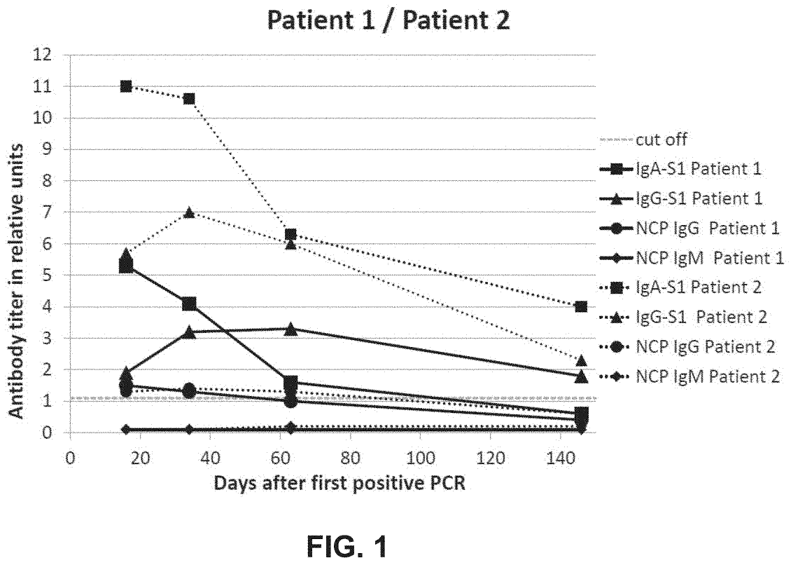

The time course of antibody levels to SEQ ID NO: 1 (IgA, IgG) and N protein (IgG, IgM) was monitored in two patients (FIG. 1). Both presented with a mild course of the disease, but the infection had been confirmed by RT-PCR, Typical specific symptoms such as temporary loss of smell were observed. EUROIMMUN products El 2606-9601 A and El 2606-9601 G (S1 protein as antigen, IgA or IgG detection, respectively) and El 2606-9601-2 G and El 2606-9601-2 M (N protein as antigen, IgG or IgM detection, respectively) were used according to the manufacturer's instructions, which are incorporated here by reference. The principle and major components of the tests are outlined in Example 2; however, different antigens (N protein as antigen instead of a polypeptide comprising SEQ ID NO: 1) as well as different secondary antibodies (binding to IgA, IgG or IgM) were used depending on which antibody was to be detected. Additional information is available in the manufacturer's instructions of El 2606-9601 A, El 2606-9601 G, El 2606-9601-2 G and El 2606-9601-2 M (EUROIMMUN).

The course of the disease was monitored over more than four months. IgA and IgG to SEQ ID NO: 1 and IgG, but no IgM to N protein were detectable initially. In both patients, at least one of IgA or IgG to SEQ ID NO: 1 was detectable after four months, whereas IgG to N protein was absent or only weak as early as two months after the first positive PCR.

These results show that antibodies to SEQ ID NO: 1 persist at least in some patients longer than those to N protein do. The level of IgA antibodies decreases more rapidly than the IgG antibody level, but IgA antibodies may still be predominant as they disappear, owing to their initially higher signal.

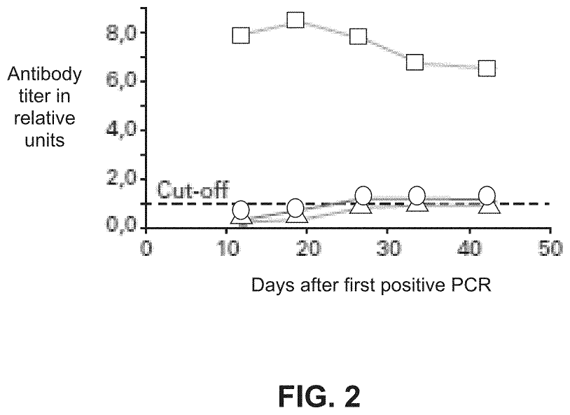

The monitoring of another time course with a third patient is shown in FIG. 2. Essentially, IgA class antibodies to SEQ ID NO: 1 (squares), IgG class antibodies to SEQ ID NO: 1 (triangles) and IgG class antibodies to N protein (circles) were determined using the same methods. As evident from FIG. 2, IgA class antibodies to S1 [SEQ ID NO: 1] were detectable over the entire time period monitored, i.e. even after 40 days after RT-PCR-confirmed SAR-CoV-2 infection, whereas levels of IgG class antibodies to either SEQ ID NO: 1 or to SARS-CoV-2 N protein remained under (or close to) the cut-off. Thus, these data additionally confirm the superior sensitivity of the instant assay which is based on the specific detection of the IgA class antibodies bound to the SEQ ID NO: 1 antigen.

### Example 4: Chemiluminescence-Based Assay for Detection of Antibodies to SEQ ID NO: 1 Used to Monitor Various Antibodies Over Time

A EUROIMMUN Random Access RA 10 Analyzer (YG 0710-0101) was used according to the manufacturer's instructions and default settings, including a reagent cartridge (LS 1254-10010 G).

Tosylactivated paramagnetic beads (M-280, lnvitrogen) were coated using the manufacturer's instructions. Briefly, beads were washed in coating buffer (0.1 M sodium phosphate pH 7.4) for 10 minutes at 37 ° C. on an IKA Roller 10 (supplied by VWR), followed by magnetic concentration of beads and removal of supernatant. Recombinant polypeptide (SEQ ID NO: 3) purified according to Example 1 was added in the same buffer, followed by addition of 3 M ammonium sulphate and incubation under the same conditions for 19 h, followed by two washing steps using washing buffer (PBS pH 7.4 0.1% BSA 0.2% Tween-20), blocking in the same buffer for 4 h at 37 ° C. (PBS pH 7.4 0.1% BSA 0,2% Tween-20) and two additional wash steps. Beads were stored in the same buffer for at least 16 h at 4° C.

The assay was carried out by mixing paramagnetic beads with sample buffer (BSA/Tween-20 in Tris-HCl EDTA pH 7. 20 μl suspension of beads (1 mg/ml) was contacted with 5 μl of sample (i.e, a blood sample of a patient) in a total of 200 μl in sample buffer. After incubation for ten minutes, beads were washed thrice in sample buffer, followed by addition of 160 μl IgG/IgA conjugate (EUROIMMUN Medizinische Labordiagnostika AG, LK 0711-10010, essentially an IgG or IgA-specific secondary antibody labeled with acridinium ester) and incubation for 10 minutes at 37° C. After three washing steps, alkaline hydrogen peroxide was rapidly added and mixed to trigger the emission of light, followed by immediate luminescence detection for 10 seconds. Results are shown in Table 2:

| | | ELISA | ELISA |
|---|---|---|---|
| | | IgA | IgG |
| | | Ratio | Ratio |
| | | pos: ≥1.1 | pos: ≥1.1 |
| Nr. | | bl: 0.8-<1.1 | bf: 0.8-<1.1 |
| 1 | IgG/IgA positive | 8.2 | 2.2 |
| 2 | samples | 12.7 | 6.3 |
| 3 | | 13.1 | 5.4 |
| 4 | Negative samples | 0.3 | 0.3 |
| 5 | | 0.3 | 0.1 |
| 6 | | 0.3 | 0.0 |
| 7 | | 0.4 | 0.1 |
| 8 | | 0.5 | 0.3 |
| 15 | IgM positive | 2.3 | 2.1 |
| 16 | samples | 1.1 | 5.6 |
| 17 | | 0.9 | 6.5 |
| 20 | blank | | |

| | Chemi- | | Chemi- | | Chemi- | |
|---|---|---|---|---|---|---|
| | luminescence | | luminescence | | luminescence | |
| | IgG | | IgA | | IgM | |
| | MW | | MW | | MW | |
| | (RLU) | Ratio | (RLU) | Ratio | (RLU) | Ratio |
| | Cutoff | pos: ≥1.1 | Cutoff | pos: ≥1.1 | Cutoff | pos: ≥1.1 |
| | 40.000 | bl: 0.8-1.1 | 50.000 | bl: 0.8-1.1 | 50.000 | bl: 0.8-1.1 |
| 1 | 36.051 | 0.9 | 79.014 | 1.6 | 9.535 | 0.2 |
| 2 | 128.217 | 3.2 | 244.029 | 4.9 | 24.354 | 0.5 |
| 3 | 223.597 | 5.6 | 307.108 | 6.1 | 21.478 | 0.4 |
| 4 | 3.222 | 0.1 | 6.861 | 0.1 | 4.495 | 0.1 |
| 5 | 4.152 | 0.1 | 4.707 | 0.1 | 2.095 | 0.0 |
| 6 | 2.005 | 0.1 | 3.943 | 0.1 | 2.133 | 0.0 |
| 7 | 15.280 | 0.4 | 9.498 | 0.2 | 4.929 | 0.1 |
| 8 | 11.401 | 0.3 | 10.970 | 0.2 | 2.745 | 0.1 |
| 15 | | | | | 9.292 | 1.0 |
| 18 | | | | | 50.715 | 1.3 |
| 17 | | | | | 62.684 | 0.0 |
| 20 | | 576 | | | 1.826 | 0.0 |

These results show that antibodies to SEQ ID NO:1 can be detected using chemiluminescence. There is a good correlation with results obtained by ELISA. Therefore, chemiluminescence can be used to practice the present invention.

### Example 5: Indirect Immunefluorescence Assay (IFA) with HEK-S1 Cells Expressing SEQ ID NO: 2

IFA was conducted using slides with a biochip array of recombinant HEK293 cells expressing SARS-CoV-2 S1 protein or HEK293 control cells to demonstrate that this method may be used for the detection of antibodies to SEQ ID NO: 1.

Each biochip mosaic was incubated with 35 μL of 1:100 PBS-diluted serum samples at room temperature for 30 min, washed with PBS-Tween and immersed in PBS-Tween for 5 min. In the second step, fluorescein isothiocyanate (FITC)-labelled goat anti-human IgG (EUROIMMUN Medizinische Labordiagnostika A G, Lübeck) was applied and incubated at room temperature for 30 min, Slides were washed again with a flush of PBS-Tween and then immersed in PBS-Tween for 5 min. Slides were embedded in PBS-buffered, DABCO containing glycerol (approximately 10 μL per field) and examined by fluorescence microscopy. Alternatively, slides were incubated 30 min with PBS prior to serum incubation and bound human IgGs were detected with by 30 min incubation with anti-human-IgG-Biotin (1:200, 109-065-098, Dianova), followed by a washing step as described above and 30 min incubation with ExtrAvidin-FITC (1:2000, E2761, Sigma-Aldrich), followed by another washing step, Samples were classified as positive or negative based on the fluorescence intensity of the transfected cells in direct comparison with control-transfected cells and control samples. Results were evaluated by two independent observers using a EUROStar II microscope (EUROIMMUN Medizinische Labordiagnostika AG, Lubeck, Germany). Reagents were obtained from Merck, Darmstadt, Germany or Sigma-Aldrich, Heidelberg, Germany if not specified otherwise.

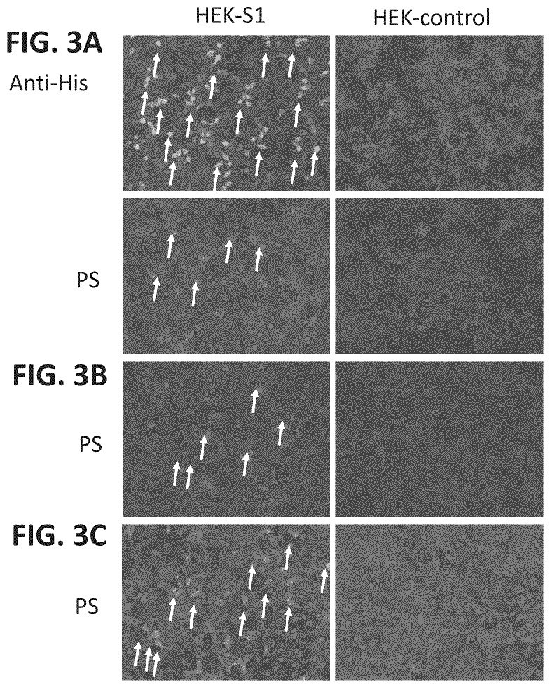

The sera of a S1 IgG ELISA-positive patient serum (PS) showed a positive reaction with S1 (SEQ ID NO: 2) but not control transfected HEK cells (FIGS. 3A-C), whereas none of the 49 S1 IgG ELISA-negative control sera reacted. Signal intensity of the patient serum was improved by incubating the HEK-S1 cells with PBS prior to serum incubation and detection of human IgG antibodies with anti-human-IgG-Biotin/ExtrAvidin-FITC. Altogether 15/24 S1 IgG ELISA-positive patient sera showed a positive reaction in HEK-S1 IFA with anti-human-IgG-Biotin/ExtrAvidin-FITC. Therefore, IFT is another method which can be used to practice the invention.

### Example 6: Vaccination Studies

A healthy subject received on day 1 an injection into the musculus quadriceps of 12.86 pg recombinant S1 protein in physiological PBS (SEQ ID NO: 3). Alum adjuvans (Twinrix for adults, EMRA-MED Arzneimittel GmbH) was applied according to the manufacturer's instructions. On days 9, 21 and 28, the subject received an injection of another 12.86 μg S1 protein in physiological PBS buffer and 10 μl Imject alaun adjuvans (Thermo Scientific Imject Alum Adjuvant Alaun) in 500 μl sodium chloride solution. Blood samples were obtained on days 10, 23 and 29.

Presence of IgG and IgA antibodies to SEQ ID NO1 (S1) and N protein was determined in serum samples from the healthy subject using serological kits (EUROIMMUN Medizinische Labordiagnostika AG, El 2606-9601 A, El 2606-9601 G, El 2606-9601-2 G and El 2606-9601-2 M, as described in Examples 2 and 3) according to the manufacturer's instructions. Determined antibody titers are shown in Table 3.

TABLE 3
Antibodies to SARS-CoV-2 antigens in
a subject vaccinated using S1 protein

| | IgA (S1) | IgG (S1) | IgG (NCP) | IgM (NCP) |
|---|---|---|---|---|
| | (Cut | (Cut | (Cut | (Cut |
| Day | off: <0.8) | off: <0.8) | off: <0.8) | off: <0.8) |
| 10 | 0.5 | 0.5 | — | — |
| 23 | 1.0 | 0.2 | — | — |
| 29 | 3.5 | 6.2 | 0.6 | 0.7 |

As positive and negative controls, samples from patients suffering from SARS-CoV-2 infection (i.e., expected presence of antibodies to SARs-CoV-2) and samples from healthy blood donors (i.e., expected absence of antibodies to SARS-CoV-2) were used: see Tables 4 and 5, respectively.

In two patients suffering from SARS-CoV-2 infection (positive controls), samples were obtained 17 and 19 days after the onset of symptoms (usually 5-6 days after the infection). Determined antibody titers are shown in Table 4.

TABLE 4
Antibodies to SARS-CoV-2 antigens in two patients
suffering from SARS-CoV-2 infection

| | IgA (S1) | IgG (S1) | IgG (N) | IgM (N) |
|---|---|---|---|---|
| | (Cut | (Cut | (Cut | (Cut |
| Day | off: <0.8) | off: <0.8) | off: <0.8) | off: <0.8) |
| Patient 1 | 2.6 | 1.8 | 1.7 | 1.3 |
| Patient 2 | 1.3 | 6.3 | 3.5 | 11.4 |

In four healthy subjects who did not receive any vaccination (negative controls), samples were obtained. Antibody titers are shown in Table 5.

TABLE 5
Antibodies to SARS-CoV-2 antigens in healthy subjects

| | IgA (S1) | IgG (S1) | IgG (NCP) | IgM (NCP) |
|---|---|---|---|---|
| | (Cut | (Cut | (Cut | (Cut |
| Day | off: <0.8) | off: <0.8) | off: <0.8) | off: <0.8) |
| Patient 1 | 0.0 | 0.1 | 0.0 | 0.5 |
| Patient 2 | 0.2 | 0.3 | 0.1 | 0.4 |
| Patient 3 | 0.1 | 0.1 | 0.1 | 0.2 |
| Patient 4 | 0.1 | 0.1 | 0.1 | 0.1 |

These results show that subjects vaccinated with SEQ ID NO: 1 or a variant thereof can be distinguished from infected subjects or subjects treated with a vaccine which does not comprise SEQ ID NO: 1 or a variant thereof by using an assay based on the detection of an antibody to SEQ ID NO: 1. While antibodies to S1 comprised in the vaccine can be detected in both, antibodies to the N protein are only detected in infected patients, but not in the vaccinated subjects.

Example 7: Detection of antibodies to RBD and SI using a dot blot Reagents: RBD (SEQ ID NO: 34) and S1 (SEQ ID NO: 3) antigens were obtained as described in Example 1. Dilutions of the samples from patients suffering from SARS-CoV-2 as shown by a positive FOR test (1075, 1076, 1078, 1079, 1080, 1084, 1085, 1098, 1099, 1100) in dilution buffer (3% Bovine Serum Albumin in 1× Universal buffer (10× concentrate, product number 20125896) were prepared. Samples from healthy blood donors (BS01, BS10, BS25, B532, BS43) were used as additional negative controls.

Monoclonal antibodies AK78, AK76 and AK80, used as positive controls, were each monoclonal anti-His tag antibodies (Lindner P, Bauer K, Krebber A, Nieba L, Kremmer E, Krebber C, Honegger A, Klinger B, Mocikat R, Plückthun A. Specific detection of his-tagged proteins with recombinant anti-His tag scFv-phosphatase or scFv-phage fusions. Biotechniques. 1997 January; 22(1):140-9).

Blot strips were made by transferring 1 pl of the antigen solution (2.69 mg ml⁻¹) on the strip. The membrane was a 0.22 μm cellulose nitrate membrane (Sartorius).

Secondary antibodies to IgA, IgG and IgM (“Anti-human-IgA-AP”, “Anti-human-IgG-AP” and “Anti-human-IgM-AP”, respectively) were from EUROIMMUN (Alkaline phosphatase-labelled anti-human IgA/G/M (goat), product no. ZD 1129 A/G/M) as was NBT/BCIP, product no. 10 123964)

Method: Blot strips were blocked by incubation for 15 minutes in washing buffer (3% Bovine Serum Albumin in 1× Universal buffer (10x concentrate product number 20125896)), followed by incubation of the strip in appropriately diluted sample for 3 hours at room temperature, followed by three washing steps using washing buffer, followed by incubation of the strip in appropriately diluted sample for another 30 minutes, followed again by the three washing steps, followed by staining by incubation in NBT/BCIP solution for ten minutes, which was stopped by washing the strips thrice thoroughly in demineralized water.

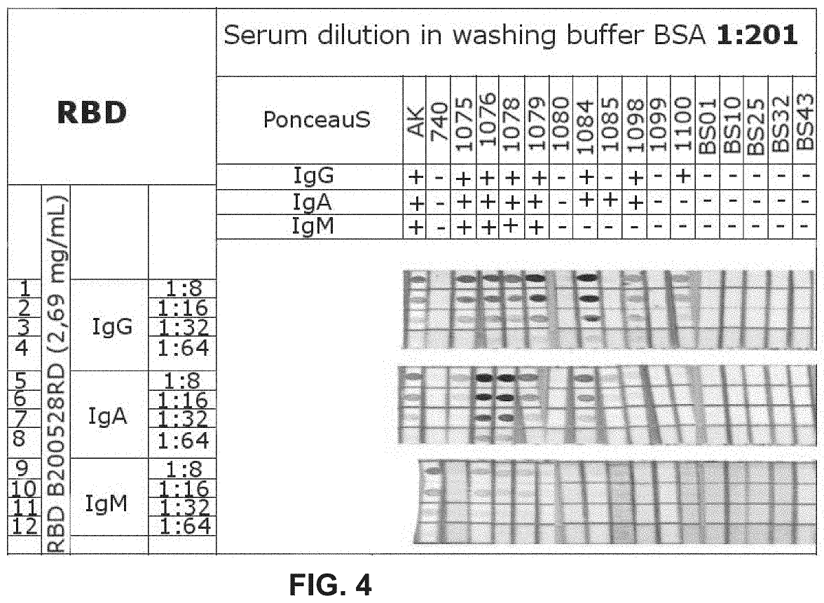

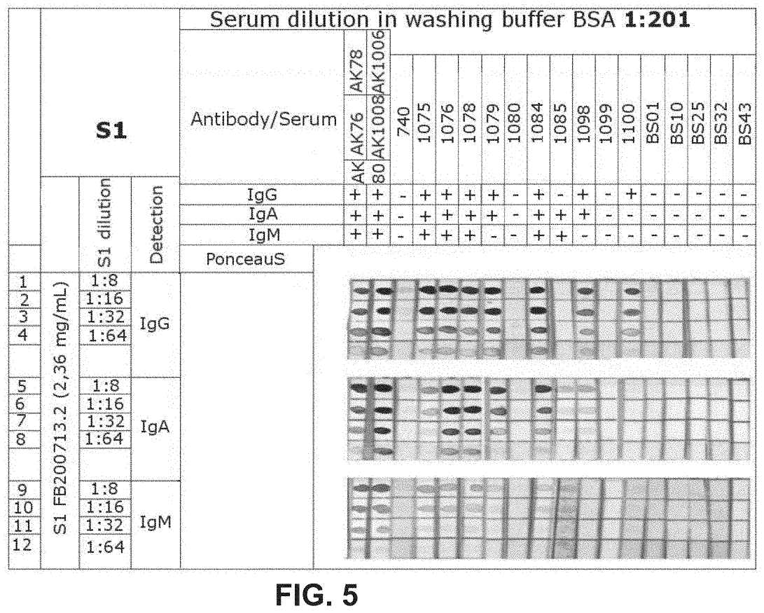

Results: FIGS. 4 and 5 show the results of dot blot analysis of the samples and controls. The assay could be established successfully as judged by positive results using the monoclonal antibodies instead of samples and no signals if negative controls were used.

While both S1 and RBD can be used to detect IgM, IgA and IgG antibodies, reactions are generally slightly stronger if S1 is used, suggesting that both the RBD and sequences flanking it contain epitopes or parts thereof. Moreover, part of the antigen-antibody interaction may be affected by the fold of the antigen.

Interestingly, IgA and IgM, but no IgG antibodies could be detected in patient 1085, confirming the ELISA-based results that detection of IgA antibodies enhances the sensitivity, particularly in addition to IgG. The patient may have been examined at an early stage of the disease when no IgG antibodies were detectable yet. Vice versa, only IgG antibodies were detectable in patient 1100, whose sample may have been obtained at a later stage of the disease, following disappearance of IgA antibodies. IgA and IgG antibodies gave generally stronger signals than IgM antibodies.

### Example 8: Detection of Antibodies to Peptides Derived from RBD and S1, Using a Dot Blot

Peptides P1 (RTQLPPAYTNS, SEQ ID NO: 41), P2 (SGTNGTKRFDN, SEQ ID NO: 42), P3 (LTPGDSSSGWTAG, SEQ ID NO: 35), P4 (NNLDSKVGG, SEQ ID NO: 55), P5 (YQAGSTPCNGV, SEQ ID NO: 36), P6 (YGFQPTNGVGYQ, SEQ ID NO: 37) and a fusion comprising P1-P6 (SEQ ID NO: 56) were expressed as N-terminal His tag fusions comprising a protease cleavage site (sequence of fusion comprising P1: SEQ ID NO: 43; P2: SEQ ID NO: 44; P3: SEQ ID NO: 45; P4: SEQ ID NO: 46; P5; SEQ ID NO: 47; P6: SEQ ID NO: 48; fusion comprising P1-P6: SEQ ID NO: 49 by E. coli Rosetta(DE3)pLacl cells using standard methods based on the pET24d plasmid, at 37° C. for 3 h in LB Medium, containing Kanamycin and Chloramphenicol, using IPTG induction. Cells were harvested, resuspended in Phosphate-Buffered Saline and stored at −20° C. until further use.

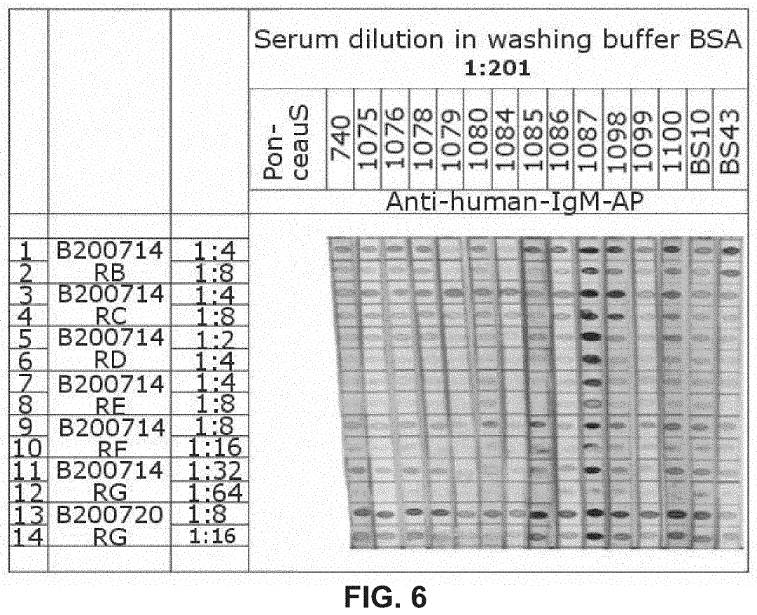

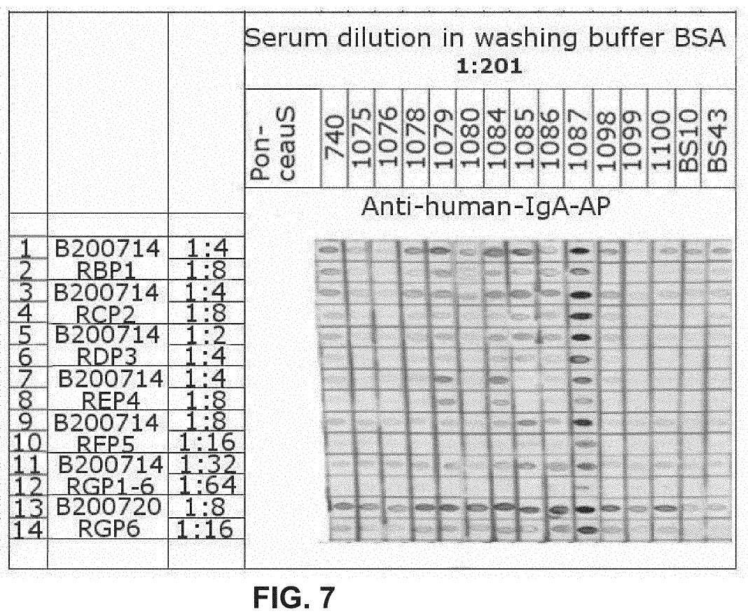

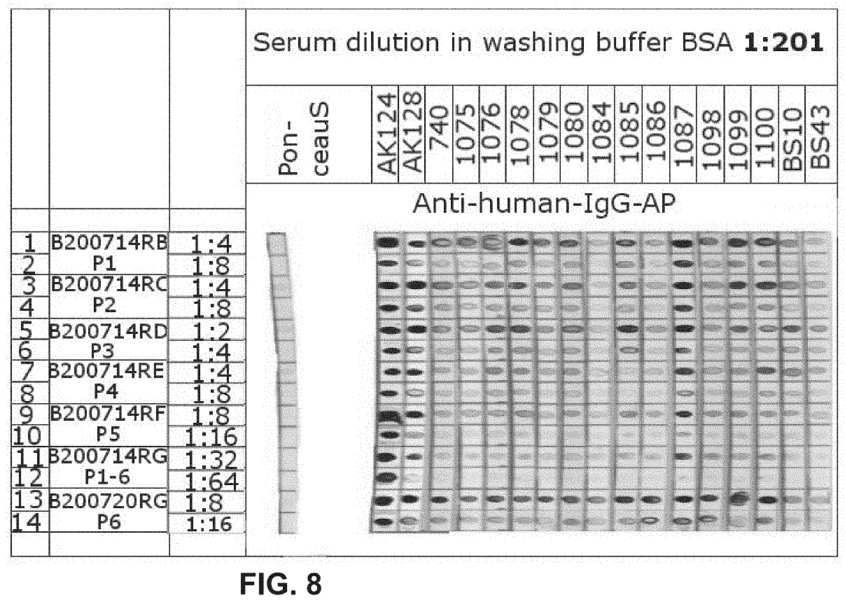

FIGS. 6, 7 and 8 show the results of the dot blot assays using the P1 to P6 constructs. P6, which is part of the RBD, showed the strongest reaction no matter whether IgA, IgM or IgG were detected, but some reactivity could also be demonstrated with P1, P2 (except for IgM), P3 and P4 (except for IgM) and P5. Reactions with IgG and IgA were generally stronger than IgM.

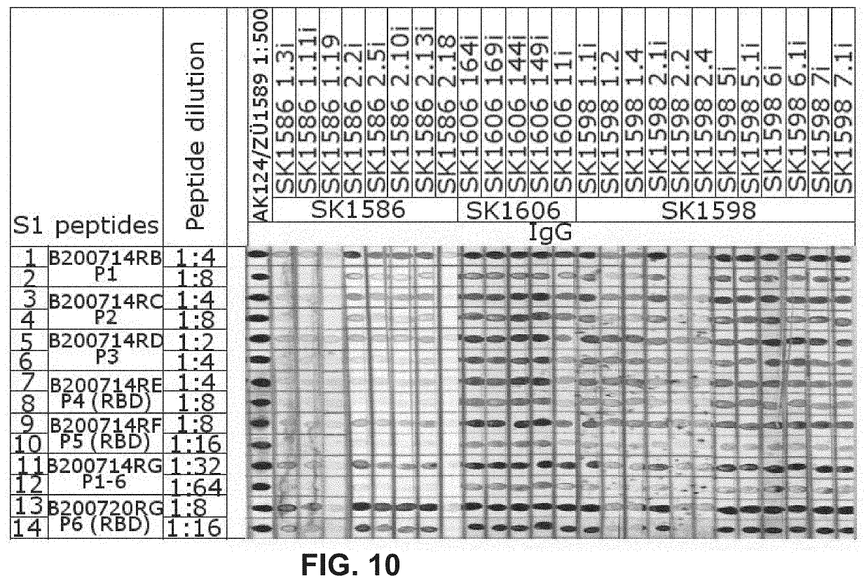

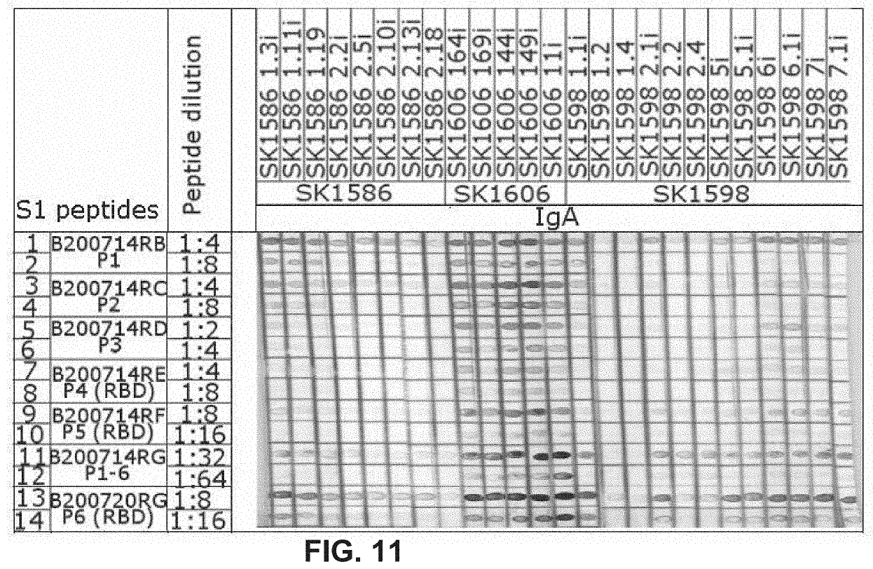

Samples representing time courses with more than one samples from several patients demonstrated that the dot blot may be used to monitor patients. For example, patient SK1586 (three time points) has the strongest IgM reaction with the first sample (13i), while the signal is weaker if the second (1.11i) and the third sample (1.19) are used, in particular with regard to P3 (FIG. 9). The same samples showed a stronger reaction if peptides P1 and P6 were used to detect IgA (FIG. 10). The reaction was generally stronger if IgG antibodies were detected, but a time-dependent reaction can be monitored using the same samples, especially peptides P6, P1 and the fusion protein comprising P1 to P6 (FIG. 11). Examples 7 and 8 show that antibodies to SEQ ID NO: 1 can be detected using dot blot and various fragments of SEQ ID NO: 1.

### Example 9: Detection of Antibodies to RBD and S1 Using a Western Blot

A non-reducing SDS PAGE was run using 2 pg of RBD (SEQ ID NO: 34) or S1 (SEQ ID NO: 3). A sample comprising 10 μl of protein was mixed with 4 μl NuPAGE-PP (NuPage LDS Sample Buffer (4×), Firma Fisher Scientific GmbH) and 1 μl of EU-PBS (Phosphate-Buffered Saline), incubated for ten minutes at 70° C. 12 μl of the resulting solution were applied per lane to a non-reducing 2D gel (NuPAGE 4-12% Bis-Tris Gel 1.0 mm×2D, Fisher Scientific GmbH) and subjected to electrophoresis in running buffer (NuPage MOPS SOS Running Buffer, Fisher Scientific GmbH). Separated Protein bands were transferred to a nitrocellulose membrane for 60 minutes at 400 mA (PowerPac HC Power Supply, Firma Bio-Rad). Membrane strips could be stained using PonceauS, followed by washing in 50 mM Tris, but were in any event subjected to blocking for 15 minutes in washing buffer (3% Bovine Serum Albumin in 1× Universal buffer (10× concentrate order no.: 20125896), followed by incubation of the strip in appropriately diluted sample for 3 hours at room temperature, followed by three washing steps using washing buffer, followed by incubation of the strip in appropriately diluted sample for another 30 minutes, followed again by the three washing steps, followed by staining by incubation in NBT/BCIP solution for ten minutes, which was stopped by washing the strips thrice thoroughly in demineralized water.

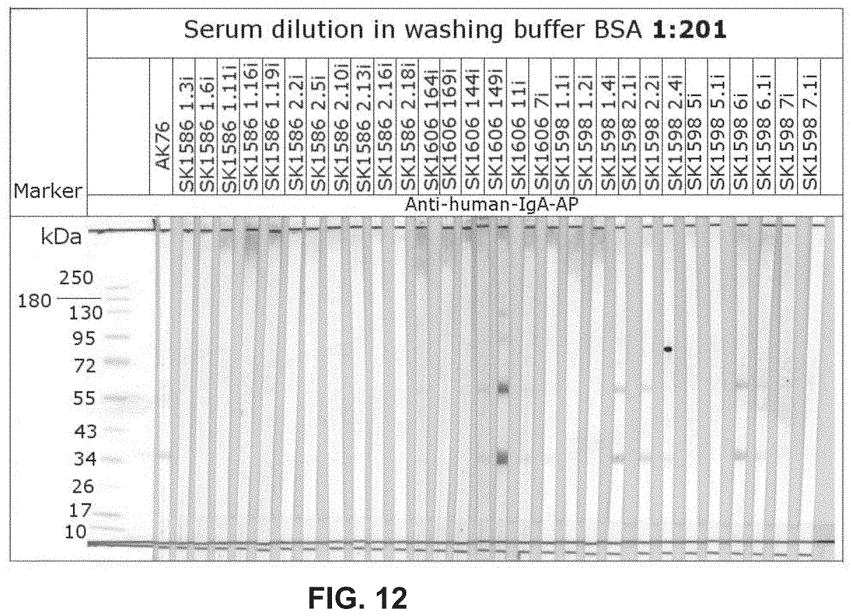

Results: Positive control (anti-His antibodies as before) and positive samples from SARS-CoV-2 infected patients show that dimers, trimers and tetramers were detected in addition to RBD monomers. A weak reaction was detected using secondary antibodies detecting IgA class antibodies with six sera, a weak reaction with another three sera and a strong reaction with one serum. If two or more samples from different time points were available and reactive, a decrease of the concentration of IgA antibodies was observed, for example with samples SK159822.1i to 2.4i, as well as with samples 6i and 61i and 7i and 7.1i (FIG. 12).

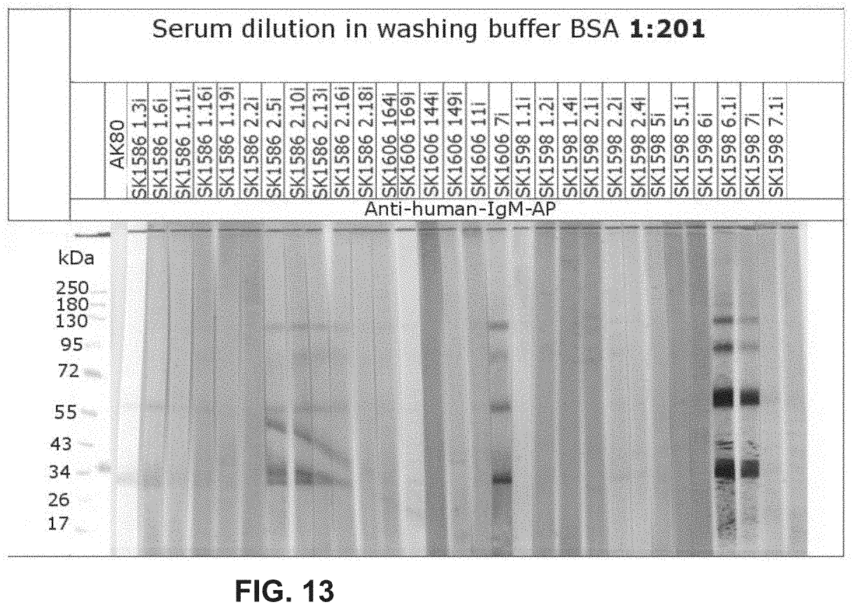

As for the detection of IgG class antibodies, all samples which were positive, as judged by ELISA, showed a reaction (FIG. 13). Sample SK1606 169i was negative based on the results of both methods.

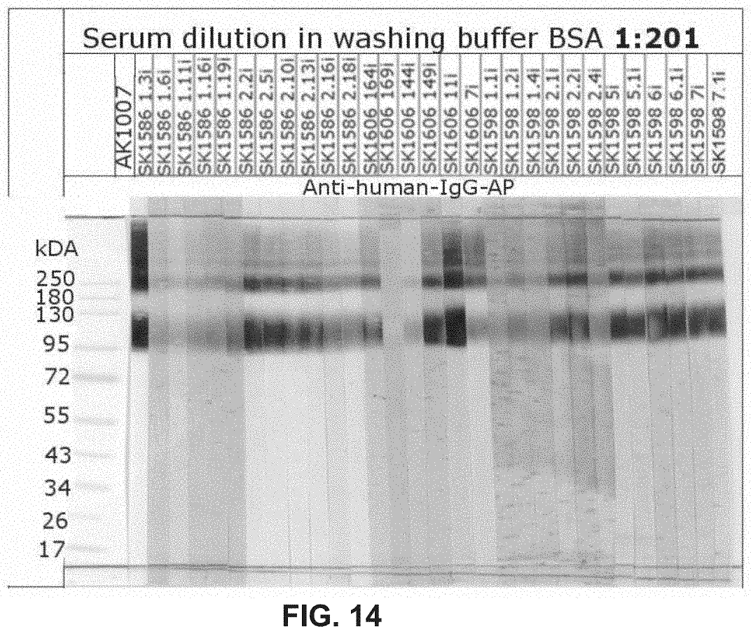

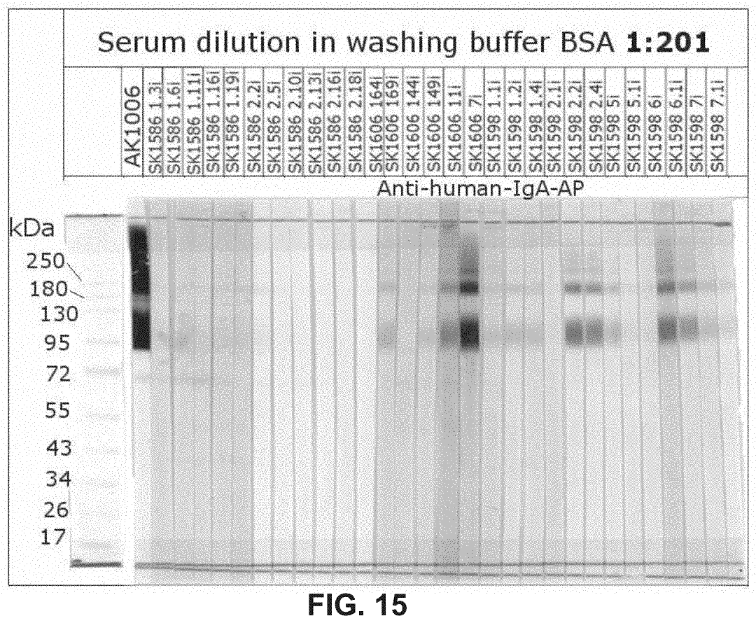

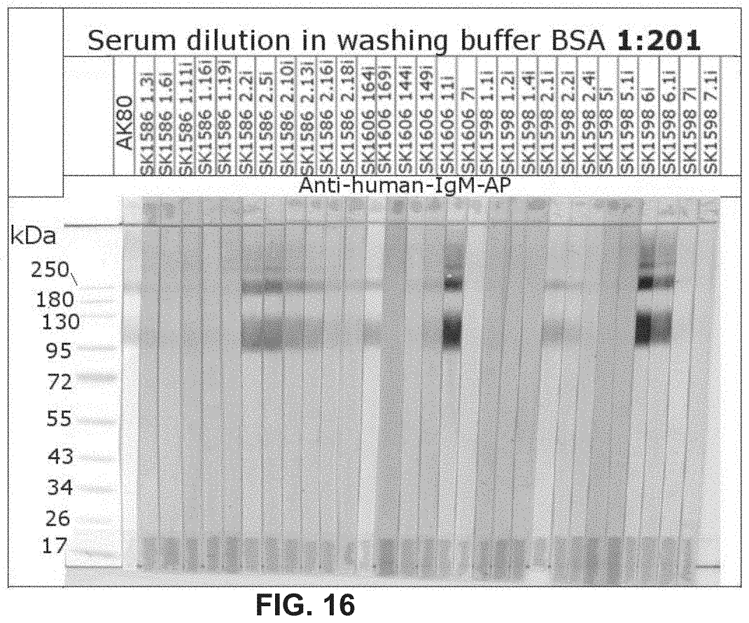

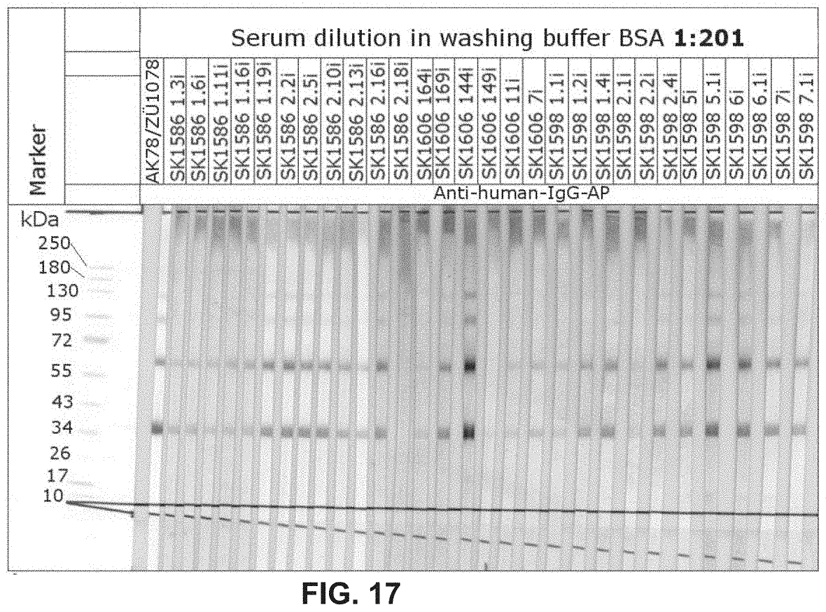

As for the detection of IgM class antibodies, a decreasing concentration of antibodies was detected in some cases, notably SK15862.2 to SK15862.16 as well as SK159986i to SK1599861i, if two or more samples from different time points were available (FIG. 14). Results were highly comparable if S1 was used rather than RBD as antigen (FIGS. 15, 16 and 17).

It is concluded that Western blotting is another method that may be used to practice the present invention.

### Example 10: Detection of IgA, IgM and IgG Antibodies to RBD Using a Competitive Test Format

A EUROIMMUN SARS-CoV-2 NeutraLISA kit (El 2606-9601-4) was used for the following experiment according to manufacturer's instructions unless specified to the contrary.

Briefly, a microtiter plate coated with the S1 domain of the spike protein of SARS-CoV-2 expressed recombinantly in the human cell line HEK293 (SEQ ID NO: 3) was used. In the first step of the analysis, the controls and samples (blood samples of a subject) were diluted with a sample buffer containing soluble biotinylated human ACE2 (SEQ ID NO: 50) and incubated in the reagent wells of a microtiter plate. Neutralizing antibodies present in the sample compete with the ACE2 for the binding sites of the coated SARS-CoV-2 S1 spike protein. Unbound ACE2 and unbound sample was removed in a subsequent washing step. To detect bound ACE2, a second incubation was performed using peroxidase-labelled streptavidin, which binds to the biotinylated ACE2 immobilized on the antigen on the microtiter plate and catalyzes a color reaction in the third step. The intensity of the produced color is inversely proportional to the concentration of neutralizing antibodies in the sample as shown in Table 6:

ACE2-Concentration 6.0 μg/ml

| | Positive | Positive | Negative |
|---|---|---|---|
| Probe | sample | sample | sample |
| 1:2.5 | 0.248 | 0.166 | 1.177 |
| 1:5 | 0.342 | 0.294 | 1.208 |
| 1:10 | 0.521 | 0.446 | 1.213 |
| 1:20 | 0.697 | 0.605 | 1.190 |
| 1:40 | 0.870 | 0.738 | 1.231 |
| 1:80 | 0.910 | 0.829 | 1.172 |
| 1:160 | 0.932 | 0.877 | 1.187 |
| Blank | 0.878 | 0.911 | 1.107 |

The results show that an increasing dilution of a positive sample, i.e. a decreasing concentration of neutralizing antibodies, leads to an increase in the produced color. By contrast, the signal is high and does not correlate with the dilution of the sample if a negative sample is used.

This confirms that the assay can be used to detect and to quantify the neutralizing antibodies against the RBD in a sample.

### Example 11: Detection of Antibodies to SARS-CoV-2 Antigens S1 and N Proteins by ELISA

A panel comprising several samples taken at different time points after the infection from each of 43 German COVED-19 patients was used to detect the presence or absence of antibodies over time. All sera were tested for antibodies against the S1 domain (SEQ ID NO: 3) of the SARS-CoV-2 spike domain (IgA and IgG ELISA kits, EUROIMMUN, products as in Examples 2 and 3) and antibodies against the N protein (IgG and IgM ELISA kits, EUROIMMUN, as in Examples 2 and 3).

Between >10 to <21 days after the onset of illness IgG and IgA antibodies against S1 of SARS-CoV-2 were detected in 70.4% and 88.9% of the samples, while IgG and IgM against N protein were detected in 86.2% and 50%, respectively. In six patients, IgA antibody to S1 was the first antibody that could be detected, while only in two cases an antibody other than IgA to S1 was the first antibody to be detected. In 30 patients no IgM antibody to N protein could be detected at any time.

More than 60 days after the onset of illness) IgG and IgA antibodies against S1 of SARS-CoV-2 were detected in 85.1% and 80.5% of the samples, while IgG and IgM against N protein were detected in 81.4% and 0%, respectively. In four patients, IgG to Si was detectable, but not IgG to N protein. By contrast, only in one patient IgG to N protein was detectable, but not IgG to S1.

These results confirm that the detection of IgA to Si is the most sensitive assay for the early detection of an antibody response against SARS-CoV-2, more specifically its S1 protein. By contrast, the detection of IgG to S1 is particularly sensitive at a later stage of the infection. Both assays may be combined by detection of IgA to S1 and IgG to S1 in one reaction or in separate reactions for increased sensitivity, optionally in combination with additional assays for increased sensitivity over an extended period of time.

### Example 12: Detection of Antibodies to SARS-CoV-2 Antigens S1 and N Proteins by ELISA During the Late Phase of a SARS-CoV-2 Infection

Using the same methodology as in Example 11, samples from another cohort of 15 patients suffering from a SARS-CoV-2 infection taken 21 days or later after the onset of illness were obtained and tested for the presence of IgG and IgM antibodies to N protein and IgA and IgG antibodies to the S1 protein. The results are shown in Table 7:

| Assay | Positive | Borderline | negative | Sensitivity |
|---|---|---|---|---|
| NCP ELISA IgG | 13 | 0 | 2 | 86.7% |
| NCP ELISA IgM | 5 | 2 | 8 | 46.7% |
| S1 ELISA IgG | 14 | 0 | 1 | 93.3% |
| S1 ELISA IgA | 12 | 3 | 0 | 100% |

The study on this additional cohort confirms that the sensitivity of diagnosis is highest at the late phase of a SARS-CoV-2 infection if antibodies to the S1 protein are detected.

### Example 13: The Specificity of the Detection of Antibodies to SARS-CoV-2 Antigens S1 and N Proteins

The specificity of the Anti-SARS-CoV-2 ELISA (IgA, El 2606-9601 A, carried out according to manufacturer's instructions, more details in Examples 2 and 3) was determined by analyzing 210 patient samples that were positive, for instance, for antibodies against other human pathogenic coronaviruses, other pathogens or for rheumatoid factors. Additionally, 1052 samples from blood donors, children and pregnant women obtained before the occurrence of SARS-CoV-2 (before January 2020) were analyzed Results in the borderline range (n=9) were not included in the calculation of the specificity. This resulted in a specificity of 98.3% as shown in Table 8.

The specificity of the Anti-SARS-CoV-2 ELISA (IgG, El 2606-9601 G, carried out according to manufacturer's instructions, more details in Examples 2 and 3) was determined in the same manner, based on 222 positive patient samples and 1052 samples from blood donors, children and pregnant. This resulted in a specificity of 98.3% as shown in Table 8:

| | | Specific- | | | Specific- |
|---|---|---|---|---|---|
| | | ity IgA | | | ity IgG |
| | | ELISA | | | ELISA |
| Panel | n | in % | Panel | n | in % |
| Blood donors | 849 | 98.2 | Blood donors | 849 | 99.5 |
| Pregnant | 99 | 97 | Pregnant | 199 | 99.5 |
| women | | | women | | |
| Children | 104 | 100 | Children | 74 | 100 |
| Elderly | 97 | 99 | Elderly | 97 | 100 |
| people | | | people | | |
| Infections | 11 | 100 | Infections | 23 | 100 |
| with other | | | with other | | |
| human | | | human | | |
| pathogenic | | | pathogenic | | |
| coronaviruses | | | coronaviruses | | |
| Influenza | 40 | 100 | Influenza | 40 | 100 |
| (freshly | | | (freshly | | |
| vaccinated) | | | vaccinated) | | |
| Acute EBV | 22 | 90.5 | Acute EBV | 22 | 100 |
| infections & | | | infections & | | |
| heterophile | | | heterophile | | |
| antibodies | | | antibodies | | |
| Rheumatoid | 40 | 100 | Rheumatoid | 40 | |
| factors | | | factors | | |
| Total | 1262 | 98.3 | Total | 1344 | 99.6 |

1. A method, comprising:
incubating a sample from a patient having or suspected of having a SARS-CoV-2 infection with a diagnostically useful carrier, wherein the diagnostically useful carrier comprises
(a) a polypeptide that is immobilized to the diagnostically useful carrier, the polypeptide comprising the sequence of SEQ ID NO: 1, SEQ ID NO: 14_{;} SEQ ID NO: 15, SEQ ID NO: 16; SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25_{;} SEQ ID NO: 26, SEQ ID NO: 27, SEQ ID NO: 28, SEQ ID NO: 29_{;} SEQ ID NO: 31, SEQ ID NO: 35; SEQ ID NO: 36, SEQ ID NO: 37, SEQ ID NO: 41, SEQ ID NO: 42, SEQ ID NO: 55; or a variant thereof; or
(b) a secondary antibody that recognizes IgA class antibodies;
detecting binding of an IgA class antibody to the polypeptide by
(i) in the case of (a), incubating a secondary antibody comprising a detectable label with the diagnostically useful carrier; or
(ii) in the case of (b), wherein the polypeptide further comprises a detectable label, incubating the polypeptide comprising the detectable label with the diagnostically useful carrier, and detecting the detectable label.

2. The method according to claim 1, wherein the diagnostically useful carrier further comprises one or more polypeptides having the sequence selected from the group consisting of: SEQ ID NO: 6, SEQ ID NO: 7, SEQ ID NO: 8; SEQ ID NO: 9, SEQ ID NO: 10, SEQ ID NO: 11; and
detecting binding of the IgA antibody to the one or more polypeptides.

3. The method according to claim 1, wherein an IgG and/or IgM class antibody to SEQ ID NO: 1 is detected.

4. The method according to claim 1, wherein the sample is a blood sample.

5. The method according to claim 1, wherein the IgA antibody is detected using a technique selected from the group consisting of colorimetry, immunofluorescence, detection of enzymatic activity, chemiluminescence, and radioactivity.

6. The method according to claim 1, wherein an infection is detected at five or fewer days after onset of SARS-CoV-2 symptoms.

7. A kit, comprising:
a polypeptide comprising SEQ ID NO: 1 or a variant thereof,
a diagnostically useful carrier, wherein
(a) the polypeptide is coated to the diagnostically useful carrier; or
(b) the polypeptide comprises an affinity tag, wherein the diagnostically useful carrier comprises a ligand to the affinity tag; and
one or more reagents selected from the group consisting of an antibody to SEQ ID NO: 1; a washing buffer; a means for detecting an IgA class antibody selected from the group consisting of a secondary antibody that binds specifically to IgA class antibodies, a polypeptide comprising the sequence of SEQ ID NO: 1, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 31, SEQ ID NO: 35, SEQ ID NO: 36, SEQ ID NO: 37, SEQ ID NO: 41, SEQ ID NO: 42, SEQ ID NO: 55, or a variant thereof with a detectable label or an affinity tag; and a polypeptide comprising ACE2 or a variant thereof; and a dilution buffer.

8. The kit according to claim 7, wherein the diagnostically useful carrier is selected from the group consisting of a bead, a test strip, a microtiter plate, a membrane, a lateral flow device, a glass surface, a slide, a microarray and a biochip and is preferably a microtiter plate.

9. The kit according to claim 7, wherein the kit comprises two or more calibrators.

10. The kit according to claim 9, wherein each calibrator is a recombinant antibody binding to SEQ ID NO: 1.

11. The method according to claim 1, wherein the antibody is an IgA class antibody.

12. The method according to claim 2, wherein an IgG class antibody is detected.

13. The method according to claim 4, wherein the blood sample is selected from the group consisting of whole blood, serum, and plasma.

14. The method according to claim 1, wherein the secondary antibody is labeled.

15. The kit according to claim , wherein the antibody to SEQ ID NO: 1 is an IgA class antibody.

16. The kit according to claim 8, wherein the bead is a paramagnetic bead.

17. The kit according to claim 8, wherein the membrane is selected from the group consisting of a western blot, a line blot, and a dot blot.

18. The kit according to claim 8, wherein the biochip is a microtiter plate.

19. The kit according to claim 9, wherein the kit has three or more calibrators.

20. The kit according to claim 10, wherein the recombinant antibody is recognized by a secondary antibody binding to IgA class antibodies.

21. A method, comprising:
incubating a sample from a patient having or suspected of having a SARS-CoV-2 infection with a first polypeptide comprising the sequence of SEQ ID NO: 1, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 31, SEQ ID NO: 35, SEQ ID NO: 36, SEQ ID NO: 37, SEQ ID NO: 41, SEQ ID NO: 42, SEQ ID NO: 55, or a variant thereof, wherein the first polypeptide comprises a detectable label and a second polypeptide comprising the sequence of SEQ ID NO: 1, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 31, SEQ ID NO: 35, SEQ ID NO: 36, SEQ ID NO: 37, SEQ ID NO: 41, SEQ ID NO: 42, SEQ ID NO: 55, or a variant thereof, wherein the second polypeptide comprises an affinity tag;
immobilizing any complex comprising the first polypeptide, the second polypeptide, and an IgA antibody to SEQ ID NO: 1, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 31, SEQ ID NO: 35, SEQ ID NO: 36, SEQ ID NO: 37, SEQ ID NO: 41, SEQ ID NO: 42, SEQ ID NO: 55, or the variant thereof, on a diagnostically useful carrier comprising a ligand to the affinity tag; and
detecting any complex immobilized on the diagnostically useful carrier.

22. A method, comprising:
incubating a sample from a patient having or suspected of having a SARS-CoV-2 infection with a first polypeptide;
incubating the sample with the second polypeptide; and
immobilizing a complex comprising the first polypeptide and the second polypeptide, wherein the first polypeptide or the second polypeptide comprises a detectable label and the second polypeptide or first polypeptide, respectively, comprises an affinity tag, wherein immobilization occurs by contacting the complex with a diagnostically useful carrier comprising a ligand binding to the affinity tag,
wherein one of the first polypeptide or the second polypeptide comprises the sequence of SEQ ID NO: 1, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 31, SEQ ID NO: 35, SEQ ID NO: 36, SEQ ID NO: 37, SEQ ID NO: 41, SEQ ID NO: 42, SEQ ID NO: 55, or a variant thereof; and wherein an other of the first polypeptide or the second polypeptide comprises SEQ ID NO: 39 or a variant thereof.


SEQUENCE LISTING (56 sequences)

SEQ ID NO: 1 - protein, 670 aa, Artificial Sequence
  SARS-CoV-2 S1 domain from S Protein
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 2 - protein, 695 aa, Artificial Sequence
  SARS-CoV-2 S1 domain from S Protein, C-terminally his-tagged
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARLEHHHHHHHH

SEQ ID NO: 3 - protein, 680 aa, Artificial Sequence
  SARS-CoV-2 S1 domain from S Protein, C-terminally his-tagged, as after cleavage of signal peptide
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARLEHHHHHHHH

SEQ ID NO: 4 - DNA, 2088 bp, Artificial Sequence
  SARS-CoV-2 S1 domain from S Protein, nucleotide sequence encoding SEQ ID NO 2
atgttcgtattccttgttctgctgcctttggttagcagtcagtgtgtcaacctgacaactcgcacgcaactgccgccagcttacaccaactctttcacaagaggcgtctactacccggacaaagtgtttcgctcatcagtgctgcactctacacaagatttgtttctgccattcttctctaacgtaacctggtttcacgcgattcatgtgtctgggacaaatgggaccaagcgcttcgacaaccccgtgctgccattcaatgacggggtgtattttgcctccaccgagaaatccaatatcatccgaggatggattttcggtactacgctggactctaaaacgcagtctctcttgatcgttaataacgccacaaatgttgtcattaaggtgtgcgagtttcagttctgtaatgatccctttctgggtgtgtattaccacaagaataacaagtcatggatggaaagcgagtttcgcgtgtactcaagtgccaataactgcacattcgagtatgtgtcccagcctttcctgatggatctcgaaggcaaacaggggaacttcaagaatctgcgcgagttcgtgtttaagaacatcgacggttatttcaagatctacagcaaacatacacccattaacctggtcagggatctccctcagggattctccgccctggaacccttggtggacttgcccattgggattaacatcactagattccagaccctgctggcccttcaccgttcctatcttactcctggcgacagtagcagtggatggaccgcaggagcagccgcttactatgtaggctatctgcagccacggaccttcctcctcaagtacaatgaaaatggtaccataactgatgctgtggactgcgctctggatccactctccgaaactaaatgcacccttaaaagcttcacggtcgaaaagggaatctaccagacaagtaactttcgggtacaacccactgagtccatcgtgcggtttcctaacatcacaaatctctgcccctttggtgaagtgtttaacgccactaggttcgcttctgtttatgcgtggaatcggaagaggatttccaattgcgtggcagactactctgtcctgtataatagcgctagcttcagcaccttcaaatgttacggggtaagcccaactaaactgaacgacctctgttttaccaacgtgtatgccgatagctttgtcatacgaggagatgaggttcgtcagattgctcctggccaaacggggaaaatcgcagactacaactacaagcttcccgacgacttcacaggatgcgtgatcgcgtggaactcaaataatctggatagcaaggttggtggcaattataactacctgtatcgactgttcaggaaaagcaacctcaaaccctttgagcgcgacatcagcaccgagatataccaagccggttcaacaccttgcaatggggtggaagggtttaactgctatttcccacttcagagctatgggtttcagccaaccaatggagtcggctaccagccctatcgggtggtagtcctgtcctttgagctgttgcatgcgcctgccacagtctgtggccctaagaagagtacgaatctggtgaagaacaagtgcgtcaacttcaattttaacggcttgactggaacaggagttctgaccgagtccaacaagaaattccttccttttcagcagtttggaagggatatagccgacactaccgatgccgttcgggatccacagacactggagattctggacattactccgtgctcatttggcggtgtatctgtcatcacacctgggaccaatacctcaaatcaggtggctgtgctctaccaggatgtgaattgtaccgaagttccagtggcaattcatgccgatcaactgactcccacctggagagtgtacagtactggcagtaacgtgtttcagacaagagctggctgtctcataggcgcagaacacgtcaacaacagctatgagtgtgacattccgatcggcgcaggcatctgtgcatcctaccagacgcaaaccaactctcccagaagagccaggctcgagcaccaccatcaccatcaccatcactaa

SEQ ID NO: 5 - protein, 12 aa, Artificial Sequence
  possible SARS-CoV-2 S1 epitope
NLKPFERDISTE

SEQ ID NO: 6 - protein, 734 aa, Artificial Sequence
  S1 [MERS_CoV]
YVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVR

SEQ ID NO: 7 - protein, 521 aa, Artificial Sequence
  S1 [HCoV-229E]
CQTTNGLNTSYSVCNGCVGYSENVFAVESGGYIPSDFAFNNWFLLTNTSSVVDGVVRSFQPLLLNCLWSVSGLRFTTGFVYFNGTGRGDCKGFSSDVLSDVIRYNLNFEENLRRGTILFKTSYGVVVFYCTNNTLVSGDAHIPFGTVLGNFYCFVNTTIGNETTSAFVGALPKTVREFVISRTGHFYINGYRYFTLGNVEAVNFNVTTAETTDFCTVALASYADVLVNVSQTSIANIIYCNSVINRLRCDQLSFDVPDGFYSTSPIQSVELPVSIVSLPVYHKHTFIVLYVDFKPQSGGGKCFNCYPAGVNITLANFNETKGPLCVDTSHFTTKYVAVYANVGRWSASINTGNCPFSFGKVNNFVKFGSVCFSLKDIPGGCAMPIVANWAYSKYYTIGSLYVSWSDGDGITGVPQPVEGVSSFMNVTLDKCTKYNIYDVSGVGVIRVSNDTFLNGITYTSTSGNLLGFKDVTKGTIYSITPCNPPDQLVVYQQAVVGAMLSENFTSYGFSNVVELPKFFYA

SEQ ID NO: 8 - protein, 745 aa, Artificial Sequence
  S1 [HCoV-OC43]
AVIGDLKCTSDNINDKDTGPPPISTDTVDVTNGLGTYYVLDRVYLNTTLFLNGYYPTSGSTYRNMALKGSVLLSRLWFKPPFLSDFINGIFAKVKNTKVIKDRVMYSEFPAITIGSTFVNTSYSVVVQPRTINSTQDGDNKLQGLLEVSVCQYNMCEYPQTICHPNLGNHRKELWHLDTGVVSCLYKRNFTYDVNADYLYFHFYQEGGTFYAYFTDTGVVTKFLFNVYLGMALSHYYVMPLTCNSKLTLEYWVTPLTSRQYLLAFNQDGIIFNAEDCMSDFMSEIKCKTQSIAPPTGVYELNGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPRPAGVLTNHDVVYAQHCFKAPKNFCPCKLNGSCVGSGPGKNNGIGTCPAGTNYLTCDNLCTPDPITFTGTYKCPQTKSLVGIGEHCSGLAVKSDYCGGNSCTCRPQAFLGWSADSCLQGDKCNIFANFILHDVNSGLTCSTDLQKANTDIILGVCVNYDLYGILGQGIFVEVNATYYNSWQNLLYDSNGNLYGFRDYIINRTFMIRSCYSGRVSAAFHANSSEPALLFRNIKCNYVFNNSLTRQLQPINYFDSYLGCVVNAYNSTAISVQTCDLTVGSGYCVDYSKNRRSRG

SEQ ID NO: 9 - protein, 744 aa, Artificial Sequence
  S1 [HCoV-HKU1]
AVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDISSSSCQLYYSLPLVNVTINNFNPSSWNRRYGFGSFNLSSYDVVYSDHCFSVNSDFCPCADPSVVNSCAKSKPPSAICPAGTKYRHCDLDTTLYVKNWCRCSCLPDPISTYSPNTCPQKKVVVGIGEHCPGLGINEEKCGTQLNHSSCFCSPDAFLGWSFDSCISNNRCNIFSNFIFNGINSGTTCSNDLLYSNTEISTGVCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRRKRR

SEQ ID NO: 10 - protein, 702 aa, Artificial Sequence
  S1 [HCoV-NL63]
FFTCNSNANLSMLQLGVPDNSSTIVTGLLPTHWFCANQSTSVYSANGFFYIDVGNHRSAFALHTGYYDANQYYIYVTNEIGLNASVTLKICKFSRNTTFDFLSNASSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVVDVMRYNLNFSANSLDNLKSGVIVFKTLQYDVLFYCSNSSSGVLDTTIPFGPSSQPYYCFINSTINTTHVSTFVGILPPTVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYV

SEQ ID NO: 11 - protein, 657 aa, Artificial Sequence
  S1 [SARS_CoV]
SGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRL

SEQ ID NO: 12 - protein, 649 aa, Artificial Sequence
  fragment of SEQ ID NO1
NSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQT

SEQ ID NO: 13 - DNA, 29903 bp, SARS-CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 14 - protein, 15 aa, Artificial Sequence
  30-44 of SEQ ID NO1
SSVLHSTQDLFLPFF

SEQ ID NO: 15 - protein, 21 aa, Artificial Sequence
  48-68 of SEQ ID NO1
TWFHAIHVSGTNGTKRFDNPV

SEQ ID NO: 16 - protein, 57 aa, Artificial Sequence
  110-166 of SEQ ID NO1
NVVIKVCEFQFCNDPFLGVYYHKNNKSWMESEFRVYSSANNCTFEYVSQPFLMDLEG

SEQ ID NO: 17 - protein, 15 aa, Artificial Sequence
  200-214 of SEQ ID NO1
DLPQGFSALEPLVDL

SEQ ID NO: 18 - protein, 25 aa, Artificial Sequence
  226-250 of SEQ ID NO1
LLALHRSYLTPGDSSSGWTAGAAAY

SEQ ID NO: 19 - protein, 25 aa, Artificial Sequence
  256-277 of SEQ ID NO1
QPRTFLLKYNENGTITDAVDCALDP

SEQ ID NO: 20 - protein, 15 aa, Artificial Sequence
  328-342 of SEQ ID NO1
NATRFASVYAWNRKR

SEQ ID NO: 21 - protein, 15 aa, Artificial Sequence
  399-414 of SEQ ID NO1
TGKIADYNYKLPDDF

SEQ ID NO: 22 - protein, 15 aa, Artificial Sequence
  434-448 of SEQ ID NO1
YNYLYRLFRKSNLKP

SEQ ID NO: 23 - protein, 23 aa, Artificial Sequence
  550-572 of SEQ ID NO1
FGRDIADTTDAVRDPQTLEILDI

SEQ ID NO: 24 - protein, 15 aa, Artificial Sequence
  590-604 of SEQ ID NO1
SNQVAVLYQDVNCTE

SEQ ID NO: 25 - protein, 19 aa, Artificial Sequence
  632-650 of SEQ ID NO1
AGCLIGAEHVNNSYECDIP

SEQ ID NO: 26 - protein, 15 aa, Artificial Sequence
  354-368 of SEQ ID NO1
YNSASFSTFKCYGVS

SEQ ID NO: 27 - protein, 15 aa, Artificial Sequence
  622-636 of SEQ ID NO1
STGSNVFQTRAGCLI

SEQ ID NO: 28 - protein, 15 aa, Artificial Sequence
  30-44 of SEQ ID NO1
SSVLHSTQDLFLPFF

SEQ ID NO: 29 - protein, 25 aa, Artificial Sequence
  226-250 of SEQ ID NO1
LLALHRSYLTPGDSSSGWTAGAAAY

SEQ ID NO: 30 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 31 - protein, 223 aa, Artificial Sequence
  RBD, a fragment from S1 domain from SARS-CoV-2 S protein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 32 - protein, 233 aa, Artificial Sequence
  RBD, a fragment from S1 domain from SARS-CoV-2 S protein, with C-terminal His tag
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFLEHHHHHHHH

SEQ ID NO: 33 - protein, 588 aa, Artificial Sequence
  S2 domain from SARS-CoV-2 S protein
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 34 - protein, 255 aa, Artificial Sequence
  RBD as used in the examples
MKHLWFFLLLVAAPRWVLSGPMRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFLEHHHHHHHH

SEQ ID NO: 35 - protein, 13 aa, Artificial Sequence
  SEQ ID NO 1-derived peptide reactive with SARS-CoV-2 antibodies
LTPGDSSSGWTAG

SEQ ID NO: 36 - protein, 11 aa, Artificial Sequence
  SEQ ID NO1-derived peptide reactive with SARS-CoV-2 antibodies
YQAGSTPCNGV

SEQ ID NO: 37 - protein, 12 aa, Artificial Sequence
  SEQ ID NO 1-derived peptide reactive with SARS-CoV-2 antibodies
YGFQPTNGVGYQ

SEQ ID NO: 38 - protein, 255 aa, Artificial Sequence; identical to SEQ ID NO: 34
  His-tagged RBD

SEQ ID NO: 39 - protein, 805 aa, Homo sapiens [sequence omitted]

SEQ ID NO: 40 - protein, 11 aa, Artificial Sequence
  SEQ ID NO 1-derived peptide reactive with SARS-CoV-2 antibodies
RTWLPPAYTNS

SEQ ID NO: 41 - protein, 11 aa, Artificial Sequence
  SEQ ID NO 1-derived peptide reactive with SARS-CoV-2 antibodies
RTQLPPAYTNS

SEQ ID NO: 42 - protein, 11 aa, Artificial Sequence
  SEQ ID NO 1-derived peptide reactive with SARS-CoV-2 antibodies
SGTNGTKRFDN

SEQ ID NO: 43 - protein, 143 aa, Artificial Sequence
  SEQ ID NO 1-derived peptide reactive with SARS-CoV-2 antibodies
MSHHHHHHHHSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGIEGRMRTQLPPAYTNSRTQLPPAYTNS

SEQ ID NO: 44 - protein, 264 aa, Artificial Sequence
  SEQ ID NO 1-derived peptide reactive with SARS-CoV-2 antibodies with GST fusion
MSHHHHHHHHSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGIEGRMMSHHHHHHHHSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGIEGRMSGTNGTKRFDNSGTNGTKRFDN

SEQ ID NO: 45 - protein, 147 aa, Artificial Sequence
  SEQ ID NO 1-derived peptide reactive with SARS-CoV-2 antibodies with GST fusion
MSHHHHHHHHSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGIEGRMLTPGDSSSGWTAGLTPGDSSSGWTAG

SEQ ID NO: 46 - protein, 139 aa, Artificial Sequence
  SEQ ID NO 1-derived peptide reactive with SARS-CoV-2 antibodies with GST fusion
MSHHHHHHHHSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGIEGRMNNLDSKVGGNNLDSKVGG

SEQ ID NO: 47 - protein, 143 aa, Artificial Sequence
  SEQ ID NO 1-derived peptide reactive with SARS-CoV-2 antibodies with GST fusion
MSHHHHHHHHSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGIEGRMYQAGSTPCNGVYQAGSTPCNGV

SEQ ID NO: 48 - protein, 145 aa, Artificial Sequence
  SEQ ID NO 1-derived peptide reactive with SARS-CoV-2 antibodies with GST fusion
MSHHHHHHHHSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGIEGRMYGFQPTNGVGYQYGFQPTNGVGYQ

SEQ ID NO: 49 - protein, 188 aa, Artificial Sequence
  SEQ ID NO 1-derived peptide reactive with SARS-CoV-2 antibodies with GST fusion, P1-P6
MSHHHHHHHHSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGIEGRMRTQLPPAYTNSSGTNGTKRFDNLTPGDSSSGWTAGNNLDSKVGGYQAGSTPCNGVYGFQPTNGVGYQ

SEQ ID NO: 50 - protein, 773 aa, Artificial Sequence
  C-terminally His-tagged extracellular domain of human ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSLEGSGSGSHHHHHHHHGSGLNDIFEAQKIEWHE

SEQ ID NO: 51 - protein, 648 aa, Artificial Sequence
  SEQ ID NO1 with mutations of SARS-CoV-2 U.K. variant B.1.1.7
VNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIH

SEQ ID NO: 52 - protein, 650 aa, Artificial Sequence
  SEQ ID NO1 with mutations of SARS-CoV-2 South African variant B.1.351
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIP

SEQ ID NO: 53 - protein, 650 aa, Artificial Sequence
  SEQ ID NO1 with mutations of SARS-CoV-2 Brazilian variant P.1
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIP

SEQ ID NO: 54 - protein, 648 aa, Artificial Sequence
  SEQ ID NO1 with mutations of SARS-CoV-2 Mink Variant from Denmark
VNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLFRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIP

SEQ ID NO: 55 - protein, 9 aa, Artificial Sequence
  SEQ ID NO 1-derived peptide reactive with SARS-CoV-2 antibodies
NNLDSKVGG

SEQ ID NO: 56 - protein, 67 aa, Artificial Sequence
  SEQ ID NO 1-derived peptide reactive with SARS-CoV-2 antibodies with GST fusion, P1-P6
RTQLPPAYTNSSGTNGTKRFDNLTPGDSSSGWTAGNNLDSKVGGYQAGSTPCNGVYGFQPTNGVGYQ